(19) United States
(12) Patent Application Publication
(10) Pub. No.: US 2025/0243215 A1
(43) Pub. Date: Jul. 31, 2025

(54) INHIBITORS OF EPIDERMAL GROWTH FACTOR RECEPTOR

(71) Applicant: National Health Research Institutes, Miaoli County (TW)

(72) Inventors: Hsing-Pang Hsieh, Miaoli County (TW); Mu-Chun Li, Miaoli County (TW); Shu-Yu Lin, Miaoli County (TW)

(21) Appl. No.: 18/428,912

(22) Filed: Jan. 31, 2024

(51) Int. Cl. C07D 491/048; A61K 31/519; A61K 31/5377; A61P 35/00

(52) U.S. Cl. CPC C07D 491/048; A61K 31/519; A61K 31/5377; A61P 35/00

(57) ABSTRACT

A compound of Formula (I):

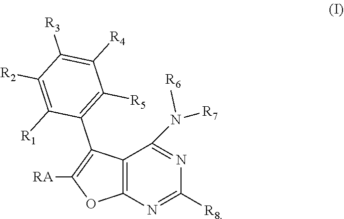

Each of R₁ to R₈ and RA is defined herein. Also disclosed are a composition containing such a compound and a method of treating cancer using the compound.

### BACKGROUND

Lung cancer is one of the leading causes of cancer deaths worldwide. The non-small cell lung cancer (NSCLC) is the most common type, accounting for around 80-85% of lung cancer cases. Oncogenic driver mutations are the main causes of the NSCLC. Among them, mutations on epidermal growth factor receptor (EGFR), a subfamily of receptor tyrosine kinases, have been extensively studied and targeted to provide effective cancer therapy.

Several drugs targeting mutated EGFR have been discovered, such as the first-generation EGFR inhibitors gefitinib and erlotinib approved by the US Food and Drug Administration in 2003 and 2004 respectively.

However, resistance to these drugs was observed in half of the treated patients due to a secondary T790M gatekeeper mutation. Second- and third-generation EGFR inhibitors were developed to overcome the T790M mutation resistance. Examples are afatinib, dacomitinib, ceritinib, and osimertinib.

Effectiveness and toxicity remain the major areas of improvement in these medicines. There is a need to develop new EGFR inhibitors as anti-cancer drugs.

### SUMMARY

To address the above need, certain compounds have been identified that are highly effective in inhibiting T790M mutant EGFR.

Accordingly, one aspect of this invention relates to furopyrimidine compounds Formula (I):

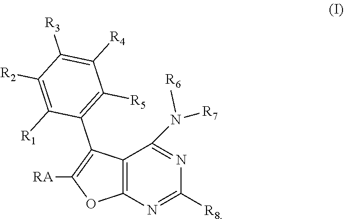

In this formula, each of R₁, R₄, R₅, and R₈, independently, is H, D, halo (e.g., F, Cl, Br, and I), or C₁-C₆ alkyl; R₂ is C₂-C₆ alkenylamido; R₃ is H, D, halo, C₁-C₆ alkyl, C₂-C₆ alkenylamido, C₁-C₆ alkylamino, C₁-C₁₀ dialkylamino, 5- to 6-membered heterocycloalkyl, C₁-C₆ alkoxy, or 5- to 6-membered heterocycloalkoxy; R₆ is H or C₁-C₆ alkyl; R₇ is C₁-C₆ alkyl, C₃-C₆ cycloalkyl, 5- to 6-membered heterocycloalkyl, aryl, C₇-C₁₀ aralkyl, heteroaryl, or C₅-C₁₀ heteroaralkyl; or R₆ and R₇, together with the nitrogen atom they bond to, form a 4- to 7-membered heterocycloalkyl; and RA is H, aryl, or heteroaryl.

Each alkyl mentioned above is unsubstituted or substituted with one or more groups selected from D, halo, hydroxyl, amino, CN, carboxyl, C₁-C₆ carboxylate, C₁-C₆alkylamino, C₁-C₁₀ dialkylamino, C₁-C₆ alkoxy, aryl, heteroaryl, aryloxy, C₅-C₆ cycloalkyl, or 5- to 6-membered heterocycloalkyl; and Each of amino, alkylamino, dialkylamino, alkenylamido, alkoxy, cycloalkyl, heterocycloalkyl, heterocycloalkoxy, aryl, aralkyl, aryloxy, heteroaryl and heteroaralkyl, independently, is unsubstituted or substituted with one or more groups selected from D, halo, hydroxyl, amino, CN, sulfamoyl, C₁-C₆ alkyl, C₁-C₆ alkoxy, C₂-C₆ alkenyl, C₂-C₆ alkynyl, C₁-C₆ alkylamino, C₁-C₁₀ dialkylamino, C₁-C₆ carboxylate, C₁-C₆ amido, 5- or 6-membered heterocycloalkyl, aryl, and heteroaryl. Any of the substituent groups can be further substituted.

A subset of compounds of Formula (I) includes compounds of Formula (II):

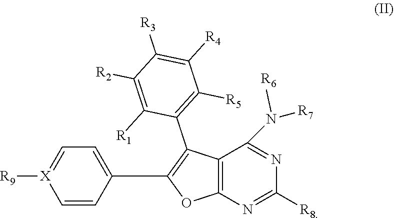

In Formula (II), X is N or C; and R₉ is absent, H, halo, or 5- to 6-membered heterocycloalkyl, provided that when X is N, R₉ is absent. R₁-R₈ are define, supra.

A further subset of compounds of Formula (I) includes compounds of Formula (III):

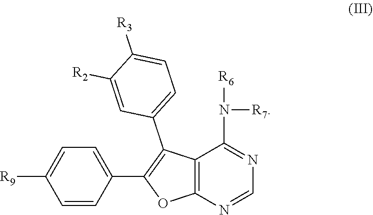

Each of R₂, R₃, R₆, R₇, R₉ is defined as above.

Preferred compounds are Compounds of Formula (IV):

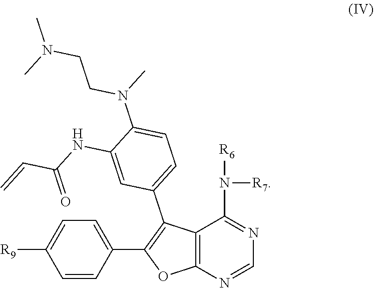

Compounds above of Formula (I), (II), (III), or (IV) can have one or more below features in any combinations:
(i) R₂ is, or

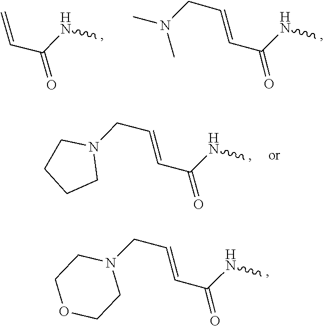

preferably

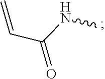

(ii) R₃ is H,

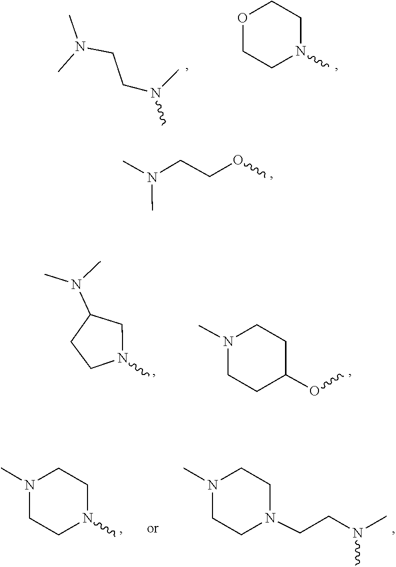

preferably,

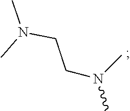

(iii) R₆ is H or CH₃, preferably H;
(iv) R₇ is C₁-C₆ alkyl, C₃-C₆ cycloalkyl, or C₇-C₁₀ aralkyl, preferably R₇ is CH₃, CH₂CH₃,

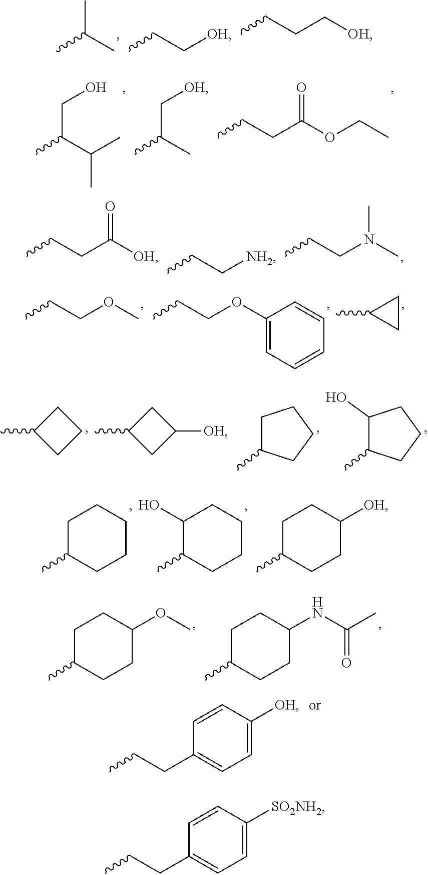

more preferably

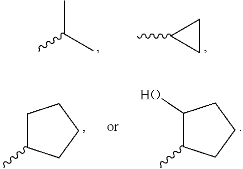

(v) R₆ and R₇, together with the nitrogen atom they bond to, are azetidinyl, pyrrolidinyl, or piperidinyl;
(vi) in formula (II), X is C;
(vii) in Formulas (II), (III), and (IV), R₉ is H, F, N(CH₃)₂,

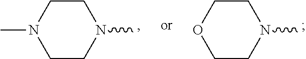

and
(viii) each of R₁, R₄, R₅, and R₈ is H.

Another aspect of this invention relates to a pharmaceutical composition, e.g., for treating cancer, comprising any one of the compounds described above and a pharmaceutically acceptable carrier.

Also within the scope of this invention is a method of treating cancer comprising administering to a subject in need thereof an effective amount of any one of the compounds, supra. Nonlimiting examples of cancer treatable by the compounds of this invention include lung cancer (e.g., the non-small cell lung cancer) and brain cancer. The current invention further includes use of such a compound (e.g., a pharmaceutical composition containing one of the compounds of Formula (I)-(IV) described above) for treating cancer or for the manufacture of a medicament for treating cancer.

The term “alkyl” herein refers to a straight or branched hydrocarbon group, containing 1-20 (e.g., 1-10 and 1-6) carbon atoms. Examples include methyl, ethyl, n-propyl, i-propyl, n-butyl, i-butyl, and t-butyl. Alkyl includes its halo substituted derivatives, i.e., haloalkyl, which refers to alkyl substituted with one or more halogen (chloro, fluoro, bromo, or iodo) atoms. Examples include trifluoromethyl, bromomethyl, and 4,4,4-trifluorobutyl. The term “alkoxy” refers to an —O-alkyl group. Examples include methoxy, ethoxy, propoxy, and isopropoxy. Alkoxy includes haloalkoxy, referring to alkoxy substituted with one or more halogen atoms. Examples include —O—CH₂Cl and —O—CHClCH₂Cl.

The term “cycloalkyl” refers to a saturated and partially unsaturated monocyclic, bicyclic, tricyclic, or tetracyclic hydrocarbon group having 3 to 12 carbons. Examples of cycloalkyl groups include, but are not limited to, cyclopropyl, cyclobutyl, cyclopentyl, cyclopentenyl, cyclohexyl, cyclohexenyl, cycloheptyl, and cyclooctyl.

The term “heterocycloalkyl” refers to a nonaromatic 5-8 membered monocyclic, 8-12 membered bicyclic, or 11-14 membered tricyclic ring system having one or more heteroatoms (e.g., O, N, P, and S). Examples of heterocycloalkyl groups include, but are not limited to, piperazinyl, imidazolidinyl, azepanyl, pyrrolidinyl, dihydrothiadiazolyl, dioxanyl, morpholinyl, tetrahydropuranyl, and tetrahydrofuranyl.

The term “aryl” refers to a 6-carbon monocyclic, 10-carbon bicyclic, 14-carbon tricyclic aromatic ring system wherein each ring may have 1 to 5 substituents. Examples of aryl groups include phenyl, naphthyl, and anthracenyl. The term “aralkyl” refers to alkyl substituted with an aryl group.

The term “heteroaryl” refers to an aromatic 5-8 membered monocyclic, 8-12 membered bicyclic, or 11-14 membered tricyclic ring system having one or more heteroatoms (e.g., O, N, P, and S). Examples include triazolyl, oxazolyl, thiadiazolyl, tetrazolyl, pyrazolyl, pyridyl, furyl, imidazolyl, benzimidazolyl, pyrimidinyl, thienyl, quinolinyl, indolyl, thiazolyl, and benzothiazolyl. The term “heteroaralkyl” refers to an alkyl group substituted with a heteroaryl group.

The terms “halo” refers to a fluoro, chloro, bromo, or iodo radical. The term “amino” refers to a radical derived from amine, which is unsubstituted or mono-/di-substituted with alkyl, aryl, cycloalkyl, heterocycloalkyl, or heteroaryl. The term “alkylamino” refers to alkyl-NH—. The term “dialkylamino” refers to alkyl-N(alkyl)-.

Compounds of Formulas (I)-(IV) can include an anion. Examples of an anion include Cl⁻, Br⁻, I⁻, So₄²⁻, PO₄³⁻, ClO₄⁻, CH₃CO₂⁻, and CF₃CO₂⁻.

The term “compound”, when referring to a compound of Formula (I), (II), (III), or (IV), also covers its salts, solvates, and prodrugs. A salt can be formed between an anion and a positively charged group (e.g., amino) on a compound; examples of a suitable anion include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. A salt can also be formed between a cation and a negatively charged group; examples of a suitable cation include sodium ion, potassium ion, magnesium ion, calcium ion, and an ammonium cation such as tetramethylammonium ion. Further, a salt can contain quaternary nitrogen atoms. A solvate refers to a complex formed between an active compound and a pharmaceutically acceptable solvent. Examples of a pharmaceutically acceptable solvent include water, ethanol, isopropanol, ethyl acetate, acetic acid, and ethanolamine. A prodrug refers to a compound that, after administration, is metabolized into a pharmaceutically active drug. Examples of a prodrug include esters and other pharmaceutically acceptable derivatives, which, upon administering to a subject, are capable of providing active compounds of this invention.

The details of the invention are set forth in the drawings, the definitions, and the detailed description below. Other features, objects, and advantages of the invention will be apparent from the following actual examples and claims.

### DETAILED DESCRIPTION OF THE INVENTION

Exemplary compounds of this invention (Compound 1-65) are summarized in Table 1 below.

TABLE 1

| | |
|---|---|
| 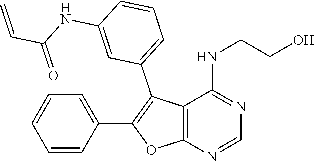 [SMILES: C=CC(=O)Nc1cccc(-c2c(-c3ccccc3)oc3ncnc(NCCO)c23)c1] | 1 |
| 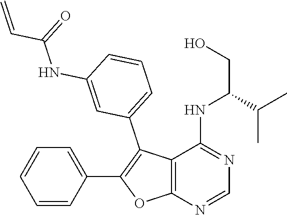 [SMILES: C=CC(=O)Nc1cccc(-c2c(-c3ccccc3)oc3ncnc(N[C@H](CO)C(C)C)c23)c1] | 2 |
| 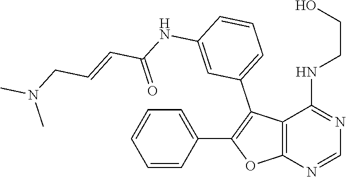 [SMILES: CN(C)C/C=C/C(=O)Nc1cccc(-c2c(-c3ccccc3)oc3ncnc(NCCO)c23)c1] | 3 |
| 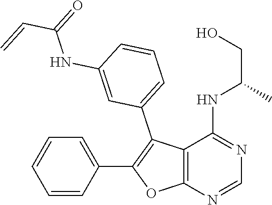 [SMILES: C=CC(=O)Nc1cccc(-c2c(-c3ccccc3)oc3ncnc(N[C@@H](C)CO)c23)c1] | 4 |
| 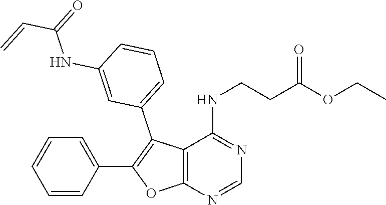 [SMILES: C=CC(=O)Nc1cccc(-c2c(-c3ccccc3)oc3ncnc(NCCC(=O)OCC)c23)c1] | 5 |
| 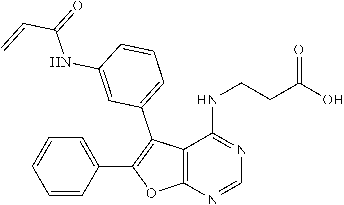 [SMILES: C=CC(=O)Nc1cccc(-c2c(-c3ccccc3)oc3ncnc(NCCC(=O)O)c23)c1] | 6 |
| 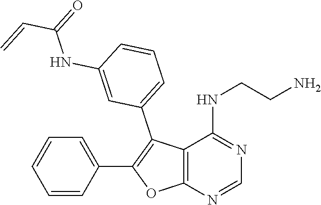 [SMILES: C=CC(=O)Nc1cccc(-c2c(-c3ccccc3)oc3ncnc(NCCN)c23)c1] | 7 |
| 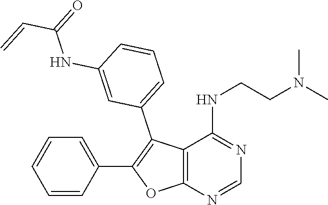 [SMILES: C=CC(=O)Nc1cccc(-c2c(-c3ccccc3)oc3ncnc(NCCN(C)C)c23)c1] | 8 |
| 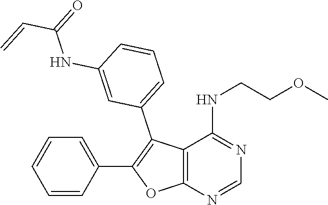 [SMILES: C=CC(=O)Nc1cccc(-c2c(-c3ccccc3)oc3ncnc(NCCOC)c23)c1] | 9 |
| 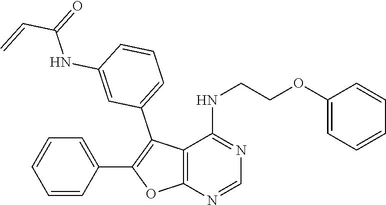 [SMILES: C=CC(=O)Nc1cccc(-c2c(-c3ccccc3)oc3ncnc(NCCOc4ccccc4)c23)c1] | 10 |
| 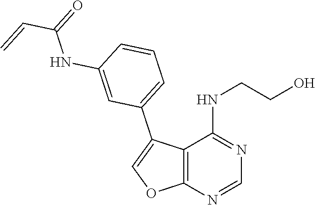 [SMILES: C=CC(=O)Nc1cccc(-c2coc3ncnc(NCCO)c23)c1] | 11 |
| 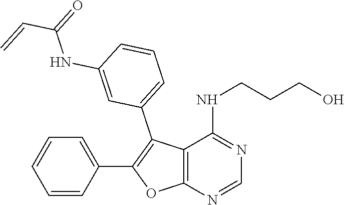 [SMILES: C=CC(=O)Nc1cccc(-c2c(-c3ccccc3)oc3ncnc(NCCCO)c23)c1] | 12 |
| 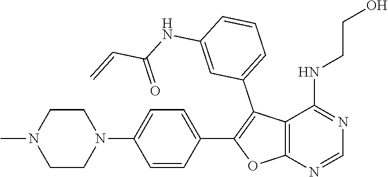 [SMILES: C=CC(=O)Nc1cccc(-c2c(-c3ccc(N4CCN(C)CC4)cc3)oc3ncnc(NCCO)c23)c1] | 13 |
| 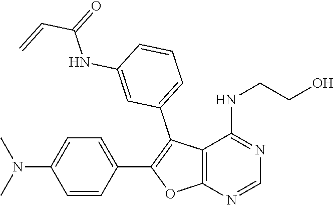 [SMILES: C=CC(=O)Nc1cccc(-c2c(-c3ccc(N(C)C)cc3)oc3ncnc(NCCO)c23)c1] | 14 |
| 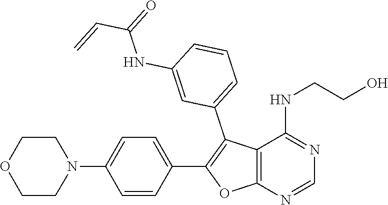 [SMILES: C=CC(=O)Nc1cccc(-c2c(-c3ccc(N4CCOCC4)cc3)oc3ncnc(NCCO)c23)c1] | 15 |
| 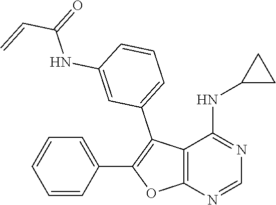 [SMILES: C=CC(=O)Nc1cccc(-c2c(-c3ccccc3)oc3ncnc(NC4CC4)c23)c1] | 16 |
| 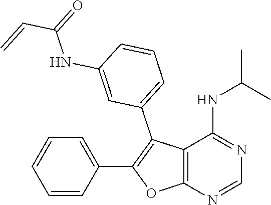 [SMILES: C=CC(=O)Nc1cccc(-c2c(-c3ccccc3)oc3ncnc(NC(C)C)c23)c1] | 17 |
| 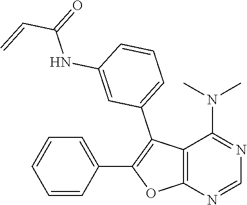 [SMILES: C=CC(=O)Nc1cccc(-c2c(-c3ccccc3)oc3ncnc(N(C)C)c23)c1] | 18 |
| 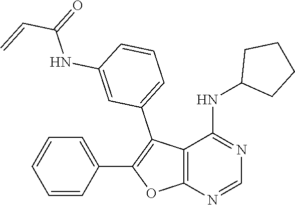 [SMILES: C=CC(=O)Nc1cccc(-c2c(-c3ccccc3)oc3ncnc(NC4CCCC4)c23)c1] | 19 |
| 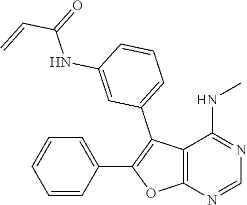 [SMILES: C=CC(=O)Nc1cccc(-c2c(-c3ccccc3)oc3ncnc(NC)c23)c1] | 20 |
| 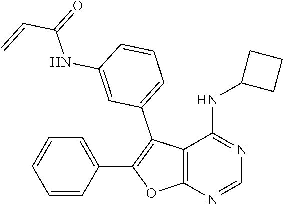 [SMILES: C=CC(=O)Nc1cccc(-c2c(-c3ccccc3)oc3ncnc(NC4CCC4)c23)c1] | 21 |
| 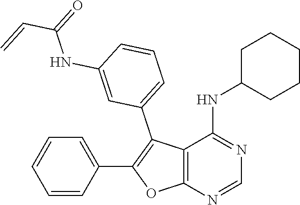 [SMILES: C=CC(=O)Nc1cccc(-c2c(-c3ccccc3)oc3ncnc(NC4CCCCC4)c23)c1] | 22 |
| 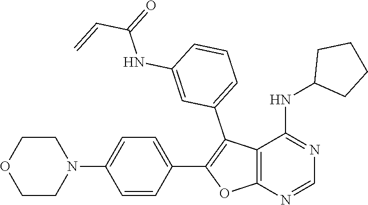 [SMILES: C=CC(=O)Nc1cccc(-c2c(-c3ccc(N4CCOCC4)cc3)oc3ncnc(NC4CCCC4)c23)c1] | 23 |
| 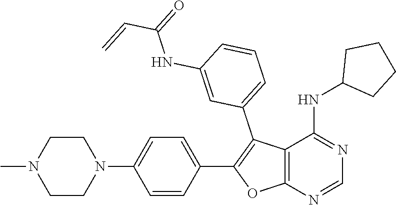 [SMILES: C=CC(=O)Nc1cccc(-c2c(-c3ccc(N4CCN(C)CC4)cc3)oc3ncnc(NC4CCCC4)c23)c1] | 24 |
| 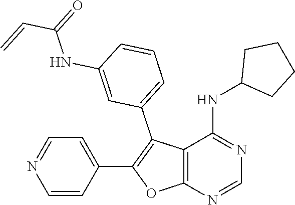 [SMILES: C=CC(=O)Nc1cccc(-c2c(-c3ccncc3)oc3ncnc(NC4CCCC4)c23)c1] | 25 |
| 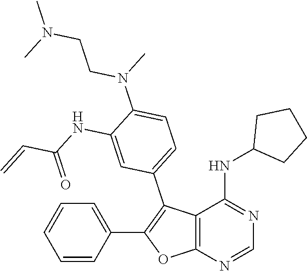 [SMILES: C=CC(=O)Nc1cc(-c2c(-c3ccccc3)oc3ncnc(NC4CCCC4)c23)ccc1N(C)CCN(C)C] | 26 |
| 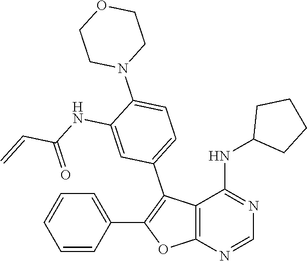 [SMILES: C=CC(=O)Nc1cc(-c2c(-c3ccccc3)oc3ncnc(NC4CCCC4)c23)ccc1N1CCOCC1] | 27 |
| 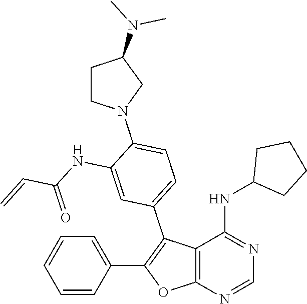 [SMILES: C=CC(=O)Nc1cc(-c2c(-c3ccccc3)oc3ncnc(NC4CCCC4)c23)ccc1N1CC[C@@H](N(C)C)C1] | 28 |
| 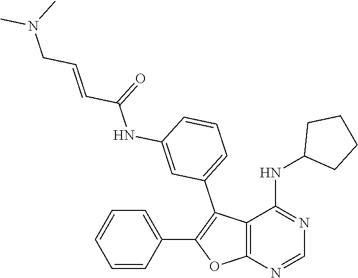 [SMILES: CN(C)C/C=C/C(=O)Nc1cccc(-c2c(-c3ccccc3)oc3ncnc(NC4CCCC4)c23)c1] | 29 |
| 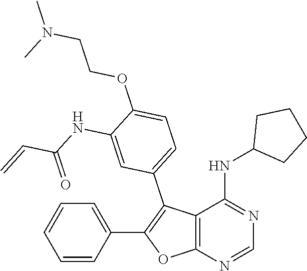 [SMILES: C=CC(=O)Nc1cc(-c2c(-c3ccccc3)oc3ncnc(NC4CCCC4)c23)ccc1OCCN(C)C] | 30 |
| 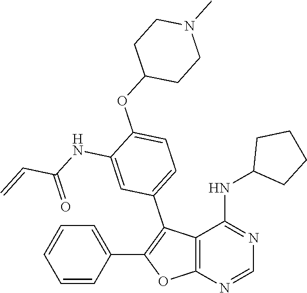 [SMILES: C=CC(=O)Nc1cc(-c2c(-c3ccccc3)oc3ncnc(NC4CCCC4)c23)ccc1OC1CCN(C)CC1] | 31 |
| 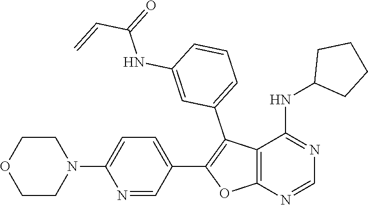 [SMILES: C=CC(=O)Nc1cccc(-c2c(-c3ccc(N4CCOCC4)nc3)oc3ncnc(NC4CCCC4)c23)c1] | 32 |
| 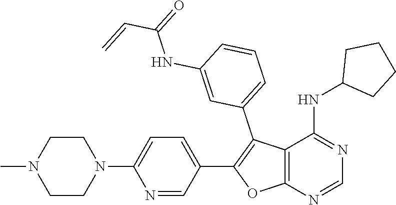 [SMILES: C=CC(=O)Nc1cccc(-c2c(-c3ccc(N4CCN(C)CC4)nc3)oc3ncnc(NC4CCCC4)c23)c1] | 33 |
| 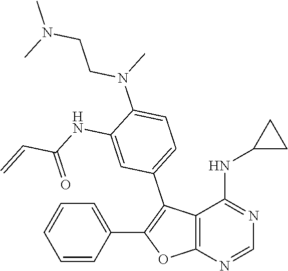 [SMILES: C=CC(=O)Nc1cc(-c2c(-c3ccccc3)oc3ncnc(NC4CC4)c23)ccc1N(C)CCN(C)C] | 34 |
| 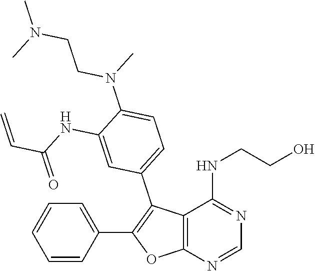 [SMILES: C=CC(=O)Nc1cc(-c2c(-c3ccccc3)oc3ncnc(NCCO)c23)ccc1N(C)CCN(C)C] | 35 |
| 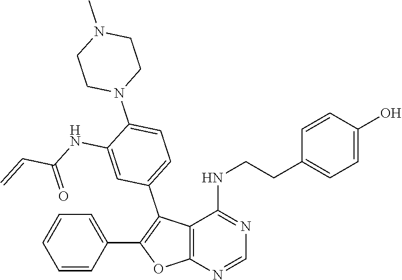 [SMILES: C=CC(=O)Nc1cc(-c2c(-c3ccccc3)oc3ncnc(NCCc4ccc(O)cc4)c23)ccc1N1CCN(C)CC1] | 36 |
| 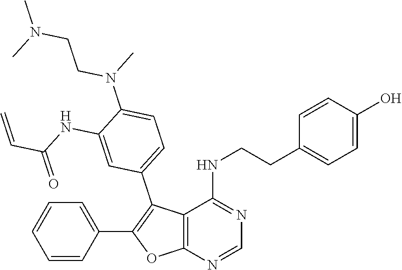 [SMILES: C=CC(=O)Nc1cc(-c2c(-c3ccccc3)oc3ncnc(NCCc4ccc(O)cc4)c23)ccc1N(C)CCN(C)C] | 37 |
| 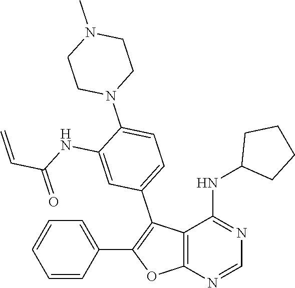 [SMILES: C=CC(=O)Nc1cc(-c2c(-c3ccccc3)oc3ncnc(NC4CCCC4)c23)ccc1N1CCN(C)CC1] | 38 |
| 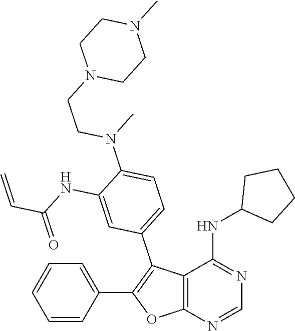 [SMILES: C=CC(=O)Nc1cc(-c2c(-c3ccccc3)oc3ncnc(NC4CCCC4)c23)ccc1N(C)CCN1CCN(C)CC1] | 39 |
| 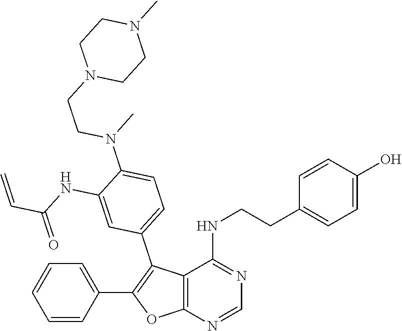 [SMILES: C=CC(=O)Nc1cc(-c2c(-c3ccccc3)oc3ncnc(NCCc4ccc(O)cc4)c23)ccc1N(C)CCN1CCN(C)CC1] | 40 |
| 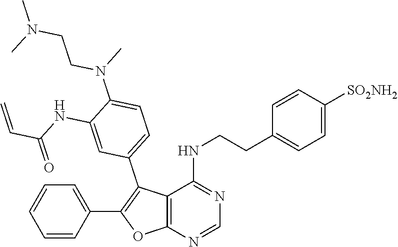 [SMILES: C=CC(=O)Nc1cc(-c2c(-c3ccccc3)oc3ncnc(NCCc4ccc(S(N)(=O)=O)cc4)c23)ccc1N(C)CCN(C)C] | 41 |
| 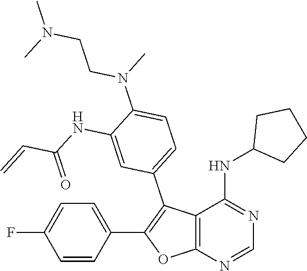 [SMILES: C=CC(=O)Nc1cc(-c2c(-c3ccc(F)cc3)oc3ncnc(NC4CCCC4)c23)ccc1N(C)CCN(C)C] | 42 |
| 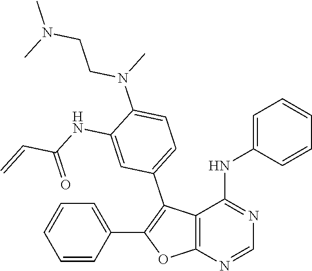 [SMILES: C=CC(=O)Nc1cc(-c2c(-c3ccccc3)oc3ncnc(Nc4ccccc4)c23)ccc1N(C)CCN(C)C] | 43 |
| 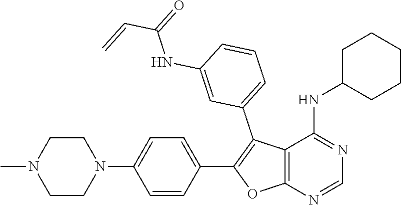 [SMILES: C=CC(=O)Nc1cccc(-c2c(-c3ccc(N4CCN(C)CC4)cc3)oc3ncnc(NC4CCCCC4)c23)c1] | 44 |
| 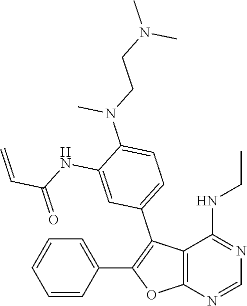 [SMILES: C=CC(=O)Nc1cc(-c2c(-c3ccccc3)oc3ncnc(NCC)c23)ccc1N(C)CCN(C)C] | 45 |
| 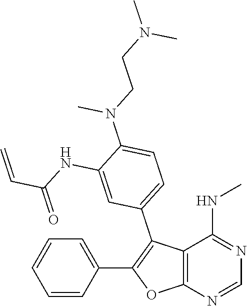 [SMILES: C=CC(=O)Nc1cc(-c2c(-c3ccccc3)oc3ncnc(NC)c23)ccc1N(C)CCN(C)C] | 46 |
| 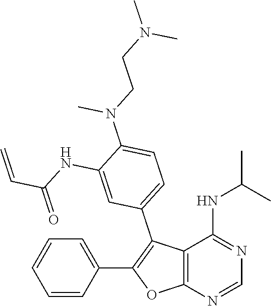 [SMILES: C=CC(=O)Nc1cc(-c2c(-c3ccccc3)oc3ncnc(NC(C)C)c23)ccc1N(C)CCN(C)C] | 47 |
| 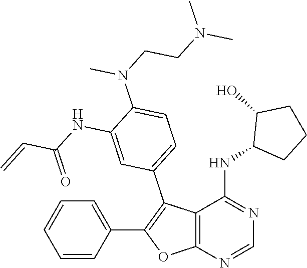 [SMILES: C=CC(=O)Nc1cc(-c2c(-c3ccccc3)oc3ncnc(N[C@H]4CCC[C@H]4O)c23)ccc1N(C)CCN(C)C] | 48 |
| 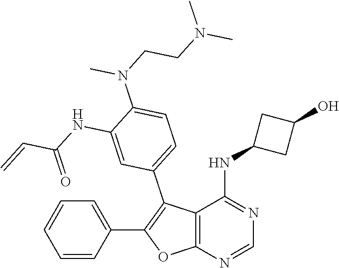 [SMILES: C=CC(=O)Nc1cc(-c2c(-c3ccccc3)oc3ncnc(N[C@H]4C[C@@H](O)C4)c23)ccc1N(C)CCN(C)C] | 49 |
| 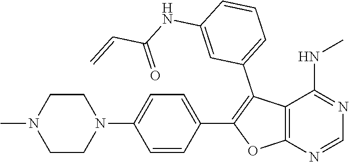 | 50 |
| 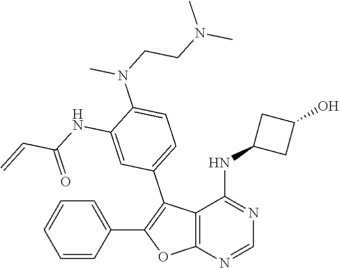 [SMILES: C=CC(=O)Nc1cc(-c2c(-c3ccccc3)oc3ncnc(N[C@H]4C[C@H](O)C4)c23)ccc1N(C)CCN(C)C] | 51 |
| 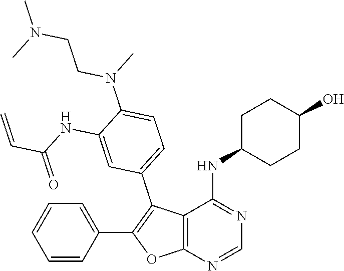 [SMILES: C=CC(=O)Nc1cc(-c2c(-c3ccccc3)oc3ncnc(N[C@H]4CC[C@@H](O)CC4)c23)ccc1N(C)CCN(C)C] | 52 |
| 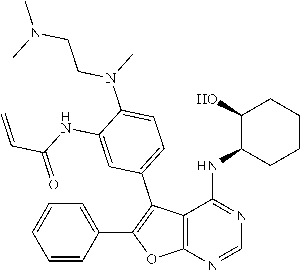 [SMILES: C=CC(=O)Nc1cc(-c2c(-c3ccccc3)oc3ncnc(N[C@@H]4CCCC[C@@H]4O)c23)ccc1N(C)CCN(C)C] | 53 |
| 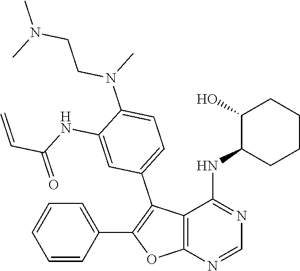 [SMILES: C=CC(=O)Nc1cc(-c2c(-c3ccccc3)oc3ncnc(N[C@@H]4CCCC[C@H]4O)c23)ccc1N(C)CCN(C)C] | 54 |
| 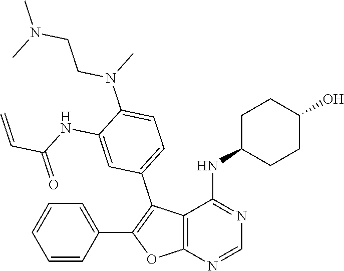 [SMILES: C=CC(=O)Nc1cc(-c2c(-c3ccccc3)oc3ncnc(N[C@H]4CC[C@H](O)CC4)c23)ccc1N(C)CCN(C)C] | 55 |
| 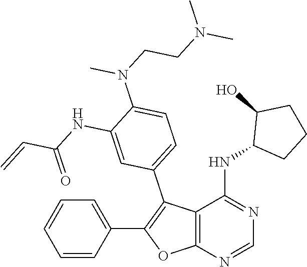 [SMILES: C=CC(=O)Nc1cc(-c2c(-c3ccccc3)oc3ncnc(N[C@H]4CCC[C@@H]4O)c23)ccc1N(C)CCN(C)C] | 56 |
| 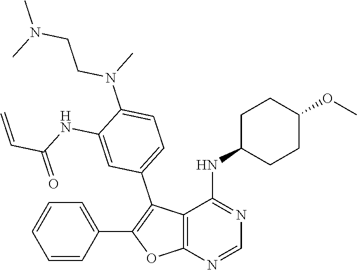 [SMILES: C=CC(=O)Nc1cc(-c2c(-c3ccccc3)oc3ncnc(N[C@H]4CC[C@H](OC)CC4)c23)ccc1N(C)CCN(C)C] | 57 |
| 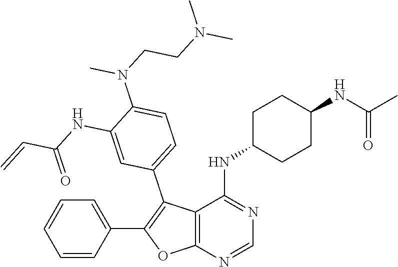 [SMILES: C=CC(=O)Nc1cc(-c2c(-c3ccccc3)oc3ncnc(N[C@H]4CC[C@H](NC(C)=O)CC4)c23)ccc1N(C)CCN(C)C] | 58 |
| 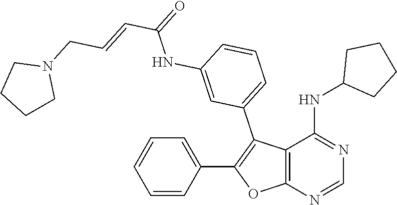 [SMILES: O=C(/C=C/CN1CCCC1)Nc1cccc(-c2c(-c3ccccc3)oc3ncnc(NC4CCCC4)c23)c1] | 59 |
| 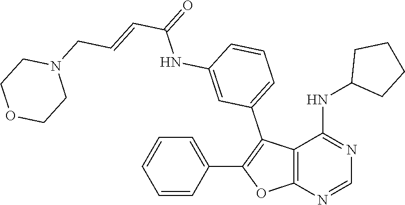 [SMILES: O=C(/C=C/CN1CCOCC1)Nc1cccc(-c2c(-c3ccccc3)oc3ncnc(NC4CCCC4)c23)c1] | 60 |
| 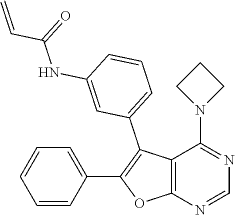 [SMILES: C=CC(=O)Nc1cccc(-c2c(-c3ccccc3)oc3ncnc(N4CCC4)c23)c1] | 61 |
| 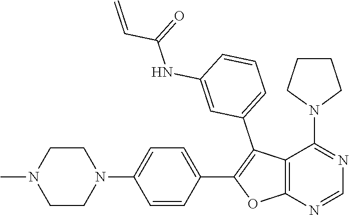 [SMILES: C=CC(=O)Nc1cccc(-c2c(-c3ccc(N4CCN(C)CC4)cc3)oc3ncnc(N4CCCC4)c23)c1] | 62 |
| 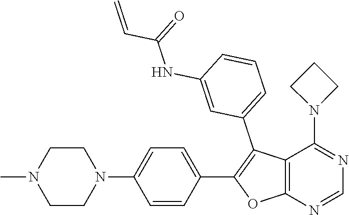 [SMILES: C=CC(=O)Nc1cccc(-c2c(-c3ccc(N4CCN(C)CC4)cc3)oc3ncnc(N4CCC4)c23)c1] | 63 |
| 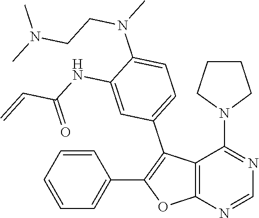 [SMILES: C=CC(=O)Nc1cc(-c2c(-c3ccccc3)oc3ncnc(N4CCCC4)c23)ccc1N(C)CCN(C)C] | 64 |
| 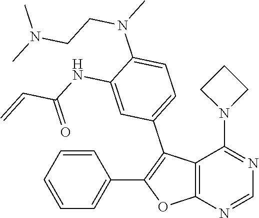 [SMILES: C=CC(=O)Nc1cc(-c2c(-c3ccccc3)oc3ncnc(N4CCC4)c23)ccc1N(C)CCN(C)C] | 65 |

Compounds of Formula (I), (II), (III), or (IV) can be prepared by conventional methods, e.g., procedures provided in the references cited above. Schemes I, II, and III below exemplify preparation of certain compounds of this invention.

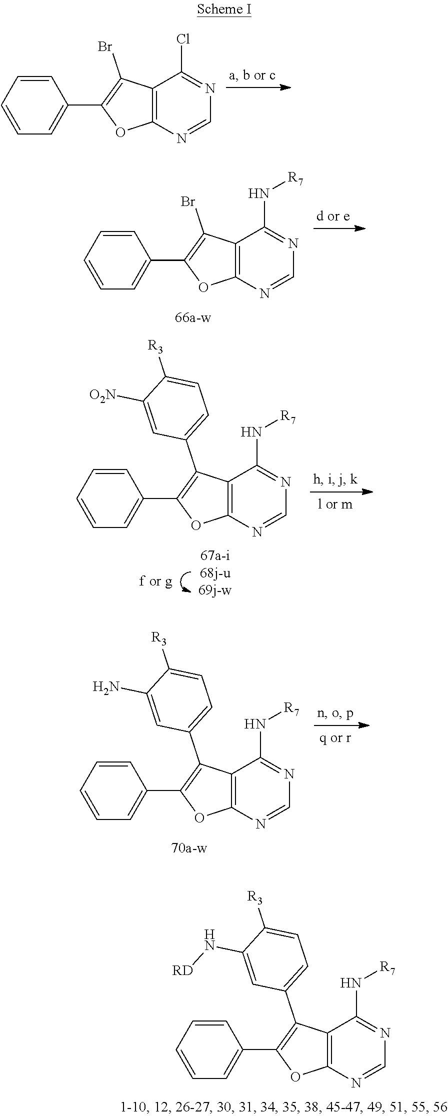

Reagents and conditions: (a) R₁NH₂, Et₃N, ethanol, reflux, 53-97%; (b) R₁NH₂, nBuOH, reflux, 62-75%; (c) R₁NH₂, DIPEA, ethanol, reflux, 92-98%; (d) R₂-boronic acids or boronic esters, Pd(dppf)Cl₂, Na₂CO₃, 1,4-dioxane, H₂O, 80° C., reflux, 2-16 h, 47-93%; (e) (3-nitrophenyl)boronic acid, Pd(dppf)Cl₂, Na₂CO₃, DMF, H₂O, 150° C., 1 h, microwave, 60%; (f) appropriate amines, Et₃N, 1,4-dioxane, 80° C., reflux, 1-12 h, 60%-quant.; (g) appropriate alcohols, NaH, THF, rt, 16 h, 46-60%; (h) H₂, Pd/C, methanol or ethanol, rt, 30-99%; (i) from 60d, (3-aminophenyl)boronic acid, Pd(dppf)Cl₂, Na₂CO₃, 1,4-dioxane, H₂O, 100° C., 16 h, 89%; (j) from 60e, (3-aminophenyl)boronic acid, Pd(dppf)Cl₂, Na₂CO₃, 1,4-dioxane, H₂O, 150° C., 1 h, microwave, 43%; (k) SnCl₂, ethanol, reflux, 1-4 h, 87-94%; (1) iron powder, ethanol, CH₂Cl₂, H₂O, sat. NH4Cl(aq.), 80° C., 0.5-16 h, 52-94%; (m) SnCl₂·2H₂O, CH₂Cl₂, methanol, reflux, 1.5 h, 31-47%; (n) acrylic acid, EDCI, CH₂Cl₂, rt, 4-16 h, 14-78%; (o) acryloyl chloride, DIPEA or Et₃N, CH₂Cl₂, rt, 1-2 h, 42-70%; (p) LiOH(aq), THF, rt, 3 h, 96%; (q) TFA, CH₂Cl₂, rt, 1 h, 96%; (r) (i) 4-bromocrotonoic acid, EDCI, CH₂Cl₂, rt, 16 h; (ii) N,N-dimethylamine, THF, rt, 4 h, 72%.

| | R₇ | R₃ | RD |
|---|---|---|---|
| 1 | 2-hydroxyethyl | H | acryloyl |
| 2 | (S)-1-hydroxy-3- | H | acryloyl |
| | methylbutan-2-yl | | |
| 3 | 2-hydroxyethyl | H | (E)-4-(dimethyl- |
| | | | amino)- |
| | | | but-2-enoyl |
| 4 | (S)-1-1-hydroxy- | H | acryloyl |
| | propan-2-yl | | |
| 5 | 3-ethoxy-3- | H | acryloyl |
| | oxopropyl | | |
| 6 | 2-carboxyethyl | H | acryloyl |
| 7 | 2-aminoethyl | H | acryloyl |
| 8 | 2-(dimethyl- | H | acryloyl |
| | animo)ethyl | | |
| 9 | 2-methoxyethyl | H | acryloyl |
| 10 | 2-phenoxyethyl | H | acryloyl |
| 12 | 3-hydroxypropyl | H | acryloyl |
| 26 | cyclopentyl | (2-(dimethylamino)ethyl)- | acryloyl |
| | | (methyl)amino | |
| 27 | cyclopentyl | morpholino | acryloyl |
| 30 | cyclopentyl | 2-(dimethylamino)ethoxy | acryloyl |
| 31 | cyclopentyl | (1-methylpiperidin-4-yl)oxy | acryloyl |
| 34 | cyclopropyl | (2-(dimethylamino)ethyl)- | acryloyl |
| | | (methyl)amino | |
| 35 | 2-hydroxyethyl | (2-(dimethylamino)ethyl)- | acryloyl |
| | | (methyl)amino | |
| 38 | cyclopentyl | 4-methylpiperazin-1-yl | acryloyl |
| 45 | ethyl | (2-(dimethylamino)ethyl)- | acryloyl |
| | | (methyl)amino | |
| 46 | methyl | (2-(dimethylamino)ethyl)- | acryloyl |
| | | (methyl)amino | |
| 47 | isopropyl | (2-(dimethylamino)ethyl)- | acryloyl |
| | | (methyl)amino | |
| 49 | (1S,3S)-3-hydroxy- | (2-(dimethylamino)ethyl)- | acryloyl |
| | cyclobutyl | (methyl)amino | |
| 51 | (1R,3R)-3-hydroxy- | (2-(dimethylamino)ethyl)- | acryloyl |
| | cyclobutyl | (methyl)amino | |
| 55 | (1S,2R)-2-hydroxy- | (2-(dimethylamino)ethyl)- | acryloyl |
| | cyclopentyl | (methyl)amino | |
| 56 | (1S,2S)-2-hydroxy- | (2-(dimethylamino)ethyl)- | acryloyl |
| | cyclopentyl | (methyl)amino | |

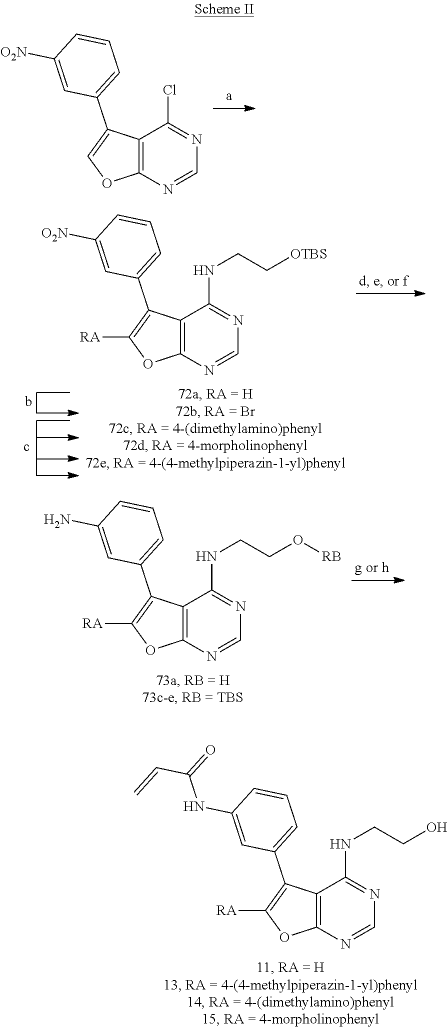

Reagents and conditions: (a) 2-[(tert-butyldimethylsilyl)oxy]ethan-1-amine, DIPEA, ethanol, reflux, 64%; (b) NBS, ACN, 100° C., microwave, 99%; (c) 4-substituted-phenylboronic acid, Pd(dppf)Cl₂CH₂Cl₂, Na₂CO₃, 100° C., 60-80%; (d) SnCl₂, HCl, CH₂Cl₂, reflux, 96%; (e) iron powder, ethanol, CH₂Cl₂, sat. NH₄Cl(aq), 80° C., 94%; (f) H₂, Pd/C, ethanol, rt, 60%; (g) acryloyl chloride, DIPEA, THF, rt, 38%; (h) acryloyl chloride, Et₃N, CH₂Cl₂, rt, then TFA, CH₂Cl₂, 34-66%.

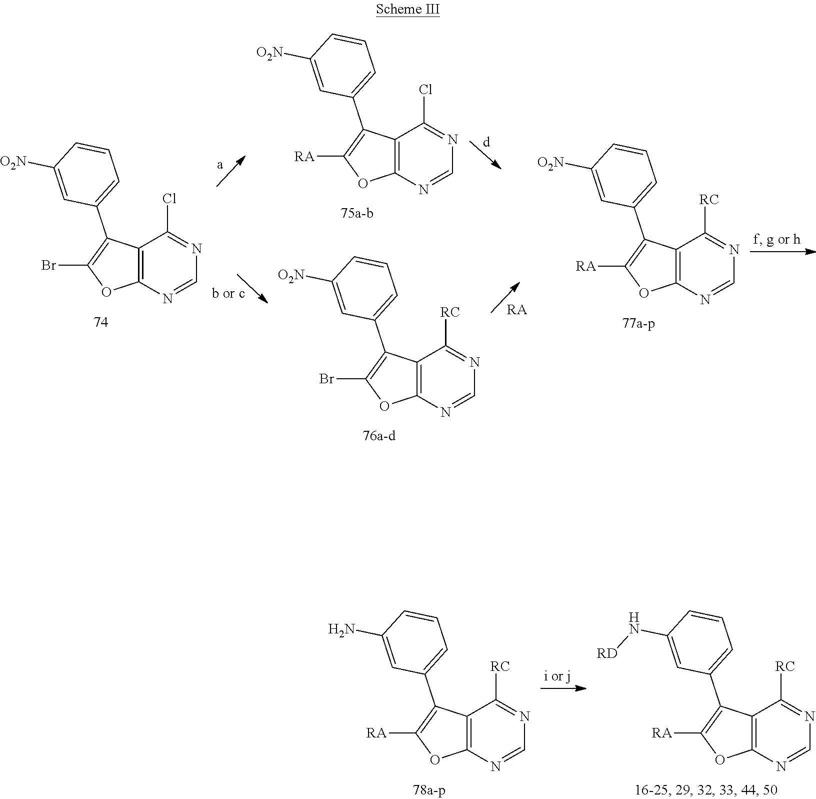

In Scheme III above, RC is R₆R₇N; and RD—NH is R₂.

Reagents and conditions: (a) appropriate boronic acids, Pd(dppf)Cl₂, Na₂CO₃, 1,4-dioxane, H₂O, 75-80° C., 3-12 h, 53-55%; (b) appropriate amines, Et₃N, ethanol or IPA, reflux, 1.5-16 h, 84-95%; (c) TBSCl, Et₃N, DMF, CH₂Cl₂, rt, 16 h, 79%; (d) appropriate amines, Et₃N or DIPEA, ethanol, reflux, 4-16 h, 51-95%; (e) appropriate boronic acids or boronic esters, Pd(dppf)Cl₂, Na₂CO₃, 1,4-dioxane, H₂O, 80° C., reflux, 1-16 h, 65-96%; (f) H₂, Pd/C, methanol, rt, 1-16 h, 83-98%; (g) SnCl₂-2H₂O, CH₂Cl₂, methanol or ethanol, 70° C., reflux, 2-4 h, 64-99%; (h) iron powder, ethanol, CH₂Cl₂, H₂O, sat. NH4Cl(aq), 80° C., 2 h, 38-85%; (i) acrylic acid, EDCI, CH₂Cl₂, rt, 1-16 h, 14-86%; (j) (i) 4-bromocrotonoic acid, EDCI, CH₂Cl₂, rt, 16 h; (ii) N,N-dimethylamine, THF, rt, 5 h, 35%.

| | RA | RC | RD |
|---|---|---|---|
| 16 | phenyl | cyclopropylamino | acryloyl |
| 17 | phenyl | isopropylamino | acryloyl |
| 18 | phenyl | dimethylamino | acryloyl |
| 19 | phenyl | cyclopetylamino | acryloyl |
| 20 | phenyl | methylamino | acryloyl |
| 21 | phenyl | cyclobutylamino | acryloyl |
| 22 | phenyl | cyclohexylamino | acryloyl |
| 23 | 4-morpholinophenyl | cyclopentylamino | acryloyl |
| 24 | 4-(4-methylpiperazin- | cyclopentylamino | acryloyl |
| | 1-yl)phenyl | | |
| 25 | pyridin-4-yl | cyclopentylamino | acryloyl |
| 29 | phenyl | cyclopetylamino | (E)-4-(dimethyl- |
| | | | amino)but-2-enoyl |
| 32 | 6-morpholinopyridin- | cyclopentylamino | acryloyl |
| | 3-yl | | |
| 33 | 6-(4-methylpiperazin- | cyclopentylamino | acryloyl |
| | 1-yl)pyridin-3-yl | | |
| 44 | 4-(4-methylpiperazin- | cyclohexylamino | acryloyl |
| | 1-yl)phenyl | | |
| 50 | 4-(4-methylpiperazin- | methylamino | acryloyl |
| | 1-yl)phenyl | | |

The compounds of the invention are useful in treating mutant EGFR mediated cancer especially the NSCLC. Their anti-cancer activities are then evaluated using known methods such as those described in actual examples below.

Some compounds of this invention contain a non-aromatic double bond or one or more asymmetric centers. Each of them occurs as a racemate or a racemic mixture, a single R enantiomer, a single S enantiomer, an individual diastereomer, a diastereometric mixture, a cis-isomer, or a trans-isomer. Compounds of such isomeric forms are within the scope of this invention. They can be present as a mixture or can be isolated using chiral synthesis or chiral separation technologies.

A compound of Formula (I), (II), (III), or (IV) is preferably formulated into a pharmaceutical composition containing a pharmaceutical carrier. The composition is then given to a subject in need thereof to treat an EGFR associated cancer.

Cancer treatable with a compound of this invention includes those caused by oncogenic mutation/overexpression of receptor tyrosine kinases such as EGFR, especially T790M mutant EGFR. Nonlimiting examples include pancreatic cancer, lung cancer, colorectal cancer, cholangiocarcinoma, multiple myeloma, melanoma, uterine cancer, endometrial cancer, thyroid cancer, acute myeloid leukemia, bladder cancer, urothelial cancer, gastric cancer, cervical cancer, head and neck squamous cell carcinoma, diffuse large B cell lymphoma, esophageal cancer, chronic lymphocytic leukemia, hepatocellular cancer, breast cancer, ovarian cancer, prostate cancer, glioblastoma, renal cancer and sarcoma, and brain metastasis. The compounds of this invention are particularly effective in treating cancer associated with EGFR such as brain cancer, lung cancer (e.g., non-small cell lung cancer, and non-small cell lung cancer with brain metastasis or leptomeningeal).

To practice the method of the present invention, a composition having one or more of the above-described furopyrimidine compounds can be administered parenterally, orally, nasally, rectally, topically, or buccally.

The term “parenteral” as used herein encompasses subcutaneous, intracutaneous, intravenous, intraperitoneal, intramuscular, intraarticular, intraarterial, intrasynovial, intrasternal, intrathecal, intralesional, and intracranial injection of a sterile injectable composition. Indeed, the term refers to any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or di-glycerides). Fatty acid, such as oleic acid and its glyceride derivatives are useful in the preparation of injectables, as are natural pharmaceutically acceptable oils, such as olive oil and castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens and Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. Oral solid dosage forms can be prepared by spray dried techniques; hot melt extrusion strategy, micronization, and nano milling technologies.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having an active compound can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound. Examples of other carriers include colloidal silicon oxide, magnesium stearate, cellulose, sodium lauryl sulfate, and D&C Yellow #10.

The term “treating” refers to application or administration of the compound to a subject with the purpose to cure, alleviate, relieve, alter, remedy, improve, or affect the disease, the symptom, or the predisposition. “An effective amount” refers to the amount of the compound which is required to confer the desired effect on the subject. Effective amounts vary, as recognized by those skilled in the art, depending on route of administration, excipient usage, and the possibility of co-usage with other therapeutic treatments such as use of other active agents. Dosage levels of a compound of Formula (I), (II), (III), or (IV) are of the order of 0.0001 mg/kg body weight to 500 mg/kg body weight (e.g., 0.05 mg/kg body weight to 300 mg/kg body weight, 0.1 mg/kg body weight to 200 mg/kg body weight, and 1 mg/kg body weight to 100 mg/kg body weight) per day. The specific dose level for a particular patient will depend upon a number of factors including age, body weight, general health, sex, diet, time of administration, rate of excretion, and the severity of the disorder. To enhance the therapeutic efficiency, the compound can be administered concomitantly with one or more of other orally active anti-cancer compounds.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever.

All publications, including patent documents, cited herein are incorporated by reference in their entirety.

### EXAMPLES

EGFR inhibitors, i.e., Compounds 1-65, were prepared and then evaluated for their anti-cancer activities.

All commercial chemicals and solvents are of reagent grade and used without further purification unless otherwise stated. All reactions were carried out under dry nitrogen or argon atmosphere and were monitored for completion by TLC using Merck® 60 F254 silica gel glass-backed plates or aluminum plates; zones were detected visually under UV irradiation (254 nm) or by spraying with potassium permanganate reagent (Aldrich®) followed by heating at 80° C.

Flash column chromatography was carried out using silica gel (Silicycle® SiliaFlash® P60, R12030B, 230-400 mesh or Merck® Grade 9385, 230-400 mesh).

¹H and ¹³C NMR spectra were recorded with Varian® Mercury-300™ or Varian® Mercury-400™ spectrometers or Bruker® 400 or 600 MHz AVANCE III™ spectrometers. Data analysis was done using Mnova® software (Mestrelab Research®). Chemical shift (δ) was reported in ppm and referenced to solvent residual signals as follows: DMSO-d₆ at 2.50 ppm, CDCl₃ at 7.26 ppm, and CD₃OD at 3.31 ppm for ¹H NMR; DMSO-d₆ at 39.5 ppm and CDCl₃ at 77.0 ppm for ¹³C NMR. Splitting patterns are indicated as follows: s=singlet; d=doublet; t=triplet; q=quartet; quin=quintet; dd=doublet of doublets; dt=doublet of triplets; dq=doublet of quartet; dquin=doublet of quintet; dset=doublet of septet; td=triplet of doublets; tt=triplet of triplets; qd=quartet of doublets, ddd=doublet of doublets of doublets; br=broad; m=multiplet. Coupling constants (J) were given in hertz (Hz).

Low-resolution mass spectra (LRMS) data were measured with an Agilent® MSD-1100 ESI-MS/MS system or Agilent Infinity 111290 LC/MS (ESI) systems. High-resolution mass spectra (HRMS) data were measured with a Varian®901-MS FT-ICR HPLC/MS-MS system. Purity of the final compounds was determined using a high-performance liquid chromatography (HPLC) system (Hitachi® 2000 series) equipped with a C18 column (Agilent® ZORBAX® Eclipse XDB-C18 5 μm. 4.6 mm×150 mm) and operating at 25° C. For method A, elution was carried out using acetonitrile as mobile phase A and water containing 0.1% formic acid+10 mmol NH₄OAc as mobile phase B. Elution conditions: at 0 min, phase A 10%+phase B 90%; at 45 min, phase A 90%+phase B 10%; at 50 min, phase A 10%+phase B 90%; at 60 min, phase A 10%+phase B 90%. For method B, elution was carried out using acetonitrile as mobile phase A and water containing 0.1% formic acid+2 mmol NH₄OAc as mobile phase B. Elution conditions: at 0 min, phase A 10%+phase B 90%; at 25 min, phase A 90%+phase B 10%; at 30 min, phase A 90%+phase B 10%; at 30.5 min, phase A 10%+phase B 90%; at 37 min, phase A 10%+ phase B 90%. The flowrate of the mobile phase was 0.5 mL/min, and the injection volume of the sample was 10 or 20 μL. Peaks were detected at 254 nm.

IUPAC nomenclature of the compounds was obtained with Mnova® software (Mestrelab Research®).

### Example 1: N-(3-{4-[(2-Hydroxyethyl)amino]-6-phenylfuro[2,3-d]pyrimidin-5-yl}phenyl)prop-2-enamide (1)

Compound 70c (see Scheme I) was prepared following the procedure described in Li et al., J. Med. Chem. 66, 2566-88 (2023), Supporting Information.

To a solution of 70c (100 mg, 0.29 mmol, 1 equiv) in dichloromethane (5 mL) were added acrylic acid (30 μL, 0.44 mmol, 1.5 equiv) and EDCI (83 mg, 0.43 mmol, 1.5 equiv); then, the reaction mixture was stirred at room temperature. After stirring for 16 h, the reaction mixture was concentrated in vacuo and purified by thin-layer chromatography (0-10% methanol in dichloromethane) to yield the title compound (75 mg, 0.19 mmol, 65%) as a white solid. ¹H NMR (600 MHz, DMSO-d₆) δ 10.32 (s, 1H), 8.34 (s, 1H), 7.86 (ddd, J=8.4, 2.4, 1.5 Hz, 1H), 7.80 (dd, J=2.4, 1.5 Hz, 1H), 7.53 (t, J=8.4 Hz, 1H), 7.48-7.44 (m, 2H), 7.38-7.30 (m, 3H), 7.24-7.21 (m, 1H), 6.43 (dd, J=16.8, 10.2 Hz, 1H), 6.26 (dd, J=16.8, 1.8 Hz, 1H), 5.77 (dd, J=10.2, 1.8 Hz, 1H), 5.50 (t, J=5.4 Hz, 1H), 4.64 (t, J=4.8 Hz, 1H), 3.49-3.41 (m, 4H). ¹³C NMR (150 MHz, DMSO-d₆) δ 164.5, 163.4, 157.2, 154.0, 145.6, 140.1, 131.8, 131.7, 130.4, 129.0, 128.8, 128.7, 127.3, 126.0, 124.4, 120.0, 119.7, 115.2, 102.1, 59.1, 42.8. LRMS (ESI) m/z: 401.2 [M+H]⁺. HRMS (ESI) m/z: calcd for C₂₃H₂₁N₄O₃, 401.1614 [M+H]⁺; found, 401.1610. HPLC purity (method A): 84.5% (t_{R}=26.43 min).

### Example 2: N-[3-(4-{[(2S)-1-Hydroxy-3-methylbutan-2-yl]amino}-6-phenylfuro[2,3-d]pyrimidin-5-yl)phenyl]prop-2-enamide (2)

Compound 70a (see Scheme I) was prepared following the procedure described in Li et al., J. Med. Chem. 66, 2566-88 (2023), Supporting Information.

To a solution of 70a (200 mg, 0.51 mmol, 1 equiv) in dichloromethane (5 mL) were added acrylic acid (40 μL, 0.58 mmol, 1.1 equiv) and EDCI (109 mg, 0.57 mmol, 1.1 equiv); then, the reaction mixture was stirred at room temperature. After stirring for 4 h, to the reaction mixture was added H₂O (10 mL), extracted with dichloromethane (10 mL×3), and washed with brine (10 mL). The combined organic layers were dried over MgSO₄, concentrated in vacuo, and purified by flash column chromatography (2-5% methanol in dichloromethane) to yield the title compound (33 mg, 0.07 mmol, 14%) as a white solid.

¹H NMR (600 MHz, DMSO-d₆) δ 10.36 (s, 1H), 8.32 (s, 1H), 7.90 (s, 1H), 7.84 (d, J=8.4 Hz, 1H), 7.56 (t, J=7.8 Hz, 1H), 7.52-7.47 (m, 2H), 7.39-7.34 (m, 2H), 7.32 (ddd, J=8.4, 7.2, 1.8 Hz, 1H), 7.27 (d, J=7.2 Hz, 1H), 6.43 (dd, J=17.1, 10.2 Hz, 1H), 6.26 (dd, J=17.1, 1.8 Hz, 1H), 5.76 (dd, J=10.2, 1.8 Hz, 1H), 4.93 (d, J=9.6 Hz, 1H), 4.64 (t, J=5.4 Hz, 1H), 4.05 (td, J=9.6, 3.6 Hz, 1H), 3.42-3.37 (m, 1H), 3.30-3.24 (m, 1H), 1.86-1.74 (m, 1H), 0.77 (d, J=6.6 Hz, 3H), 0.57 (d, J=6.6 Hz, 3H). ¹³C NMR (150 MHz, DMSO-d₆) δ 164.4, 163.4, 157.5, 154.1, 145.5, 140.3, 132.1, 131.6, 130.4, 129.0, 128.9, 128.7, 127.4, 125.8, 124.3, 119.8, 119.6, 115.1, 102.3, 60.4, 56.1, 27.7, 19.4, 16.8.

LRMS (ESI) m/z: 443.2 [M+H]⁺. HRMS (ESI) m/z: calcd for C₂₆H₂₇N₄O₃, 443.2083 [M+H]⁺; found, 443.2085.

HPLC purity (method A): 100% (t_{R}=32.42 min).

### Example 3: (2E)-4-(Dimethylamino)-N-(3-{4-[(2-hydroxyethyl)amino]-6-phenylfuro[2,3-d]pyrimidin-5-yl}phenyl)but-2-enamide (3)

To a solution of 70c (100 mg, 0.29 mmol, 1 equiv) in dichloromethane (5 mL) were added 4-bromocrotonoic acid (71 mg, 0.43 mmol, 1.5 equiv) and EDCI (83 mg, 0.43 mmol, 1.5 equiv); then, the reaction mixture was stirred at room temperature. After stirring for 16 h, the reaction mixture was concentrated in vacuo, dissolved in THF (5.0 mL), and added 2 M N,N-dimethylamine (250 μL, 0.50 mmol, 1.7 equiv) in THF. Then, the reaction mixture was stirred at room temperature for 4 h, concentrated in vacuo, and purified by thin-layer chromatography (0-15% methanol in dichloromethane) to yield the title compound (95 mg, 0.21 mmol, 72%) as a white solid.

¹H NMR (600 MHz, DMSO-d₆) δ 10.42 (s, 1H), 8.34 (s, 1H), 7.85 (d, J=8.4 Hz, 1H), 7.82 (s, 1H), 7.51 (t, J=7.8 Hz, 1H), 7.46 (d, J=6.9 Hz, 2H), 7.36 (dd, J=7.8, 6.9 Hz, 2H), 7.34-7.30 (m, 1H), 7.19 (d, J=7.8 Hz, 1H), 6.73 (dt, J=15.3, 5.7 Hz, 1H), 6.30 (d, J=15.3 Hz, 1H), 5.50 (t, J=5.4 Hz, 1H), 4.69 (s, 1H), 3.49-3.39 (m, 4H), 3.04 (d, J=5.7 Hz, 2H), 2.16 (s, 6H). ¹³C NMR (150 MHz, DMSO-d₆) δ 164.5, 163.5, 157.2, 154.0, 145.5, 141.8, 131.7, 130.3, 129.0, 128.8, 128.7, 126.0, 125.7, 124.1, 119.9, 119.6, 115.3, 102.1, 59.7, 59.1, 45.1, 42.8.

LRMS (ESI) m/z: 458.2 [M+H]⁺. HRMS (ESI) m/z: calcd for C₂₆H₂₇N₅NaO₃, 480.2012 [M+Na]⁺; found, 480.2008.

HPLC purity (method A): 97.4% (t_{R}=19.17 min).

### Example 4: N-[3-(4-{[(2S)-1-Hydroxypropan-2-yl]amino}-6-phenylfuro[2,3-d]pyrimidin-5-yl)phenyl]prop-2-enamide (4)

Compound 70b (see Scheme I) was prepared following the procedure described in Li et al., J. Med. Chem. 66, 2566-88 (2023), Supporting Information.

To a solution of 70b (20 mg, 0.06 mmol, 1 equiv) in dichloromethane (0.5 mL) were added acrylic acid (4.2 μL, 0.06 mmol, 1.1 equiv) and EDCI (12 mg, 0.06 mmol, 1.1 equiv); then, the reaction mixture was stirred at room temperature. After stirring for 16 h, the reaction mixture was concentrated in vacuo and purified by thin-layer chromatography (2-15% methanol in dichloromethane) to yield the title compound (3 mg, 0.01 mmol, 14%) as a white solid.

¹H NMR (400 MHz, DMSO-d₆) δ 10.33 (s, 1H), 8.35 (s, 1H), 7.90-7.78 (m, 2H), 7.59-7.44 (m, 3H), 7.43-7.29 (m, 3H), 7.24 (d, J=7.6 Hz, 1H), 6.42 (dd, J=17.0, 10.0 Hz, 1H), 6.25 (dd, J=17.0, 2.2 Hz, 1H), 5.77 (dd, J=10.0, 2.2 Hz, 1H), 5.18 (d, J=7.6 Hz, 1H) 4.70 (t, J=5.2 Hz, 1H), 4.23-4.08 (m, 1H), 3.32-3.27 (m, 2H), 1.02 (d, J=6.8 Hz, 3H). ¹³C NMR (150 MHz, DMSO-d₆) δ 164.5, 163.4, 156.7, 154.1, 145.5, 140.1, 131.9, 131.6, 130.3, 129.0, 128.8, 128.7, 127.3, 126.0, 124.3, 119.9, 119.6, 115.1, 102.1, 63.5, 47.6, 17.0.

LRMS (ESI) m/z: 415.2 [M+H]⁺. HRMS (ESI) m/z: calcd for C₂₄H₂₂N₄NaO₃, 437.1590 [M+Na]⁺; found, 437.1587.

HPLC purity (method A): 98.1% (t_{R}=28.29 min).

### Example 5: Ethyl 3-({5-[3-(Acryloylamino)phenyl]-6-phenylfuro[2,3-d]pyrimidin-4-yl}amino)propanoate (5)

Compound 70 g (see Scheme I) was prepared following the procedure described in Li et al., J. Med. Chem. 66, 2566-88 (2023), Supporting Information.

To a solution of 70 g (400 mg, 0.99 mmol, 1 equiv) in dichloromethane (10 mL) at 0° C. were added triethylamine (170 μL, 1.22 mmol, 1.2 equiv) and a solution of acryloyl chloride (99 μL, 1.23 mmol, 1.2 equiv) in dichloromethane (5.0 mL); then, the reaction mixture was stirred at room temperature. After stirring for 1 h, the reaction mixture was concentrated in vacuo and purified by CombiFlash automated flash chromatography (2% methanol in dichloromethane) to yield the title compound (261 mg, 0.57 mmol, 58%) as a brown solid.

¹H NMR (600 MHz, DMSO-d₆) δ 10.33 (s, 1H), 8.36 (s, 1H), 7.83 (ddd, J=8.4, 2.4, 1.5 Hz, 1H), 7.80 (dd, J=1.8, 1.5 Hz, 1H), 7.52 (t, J=7.8 Hz, 1H), 7.47-7.43 (m, 2H), 7.38-7.30 (m, 3H) 7.18-7.15 (m, 1H), 6.43 (dd, J=17.1, 10.2 Hz, 1H), 6.35 (dd, J=17.1, 1.8 Hz, 1H), 5.77 (dd, J=10.2, 1.8 Hz, 1H), 5.55 (t, J=6.0 Hz, 1H), 3.96 (q, J=7.2 Hz, 2H), 3.63 (td, J=6.0, 6.0 Hz, 2H), 2.52 (t, J=6.6 Hz, 2H), 1.09 (t, J=7.2 Hz, 3H). ¹³C NMR (150 MHz, DMSO-d₆) δ 171.5, 164.6, 163.4, 156.9, 154.0, 145.7, 140.1, 131.7, 131.6, 130.3, 129.0, 128.84, 128.79, 127.3, 126.1, 124.5, 120.0, 119.7, 115.1, 102.3, 60.0, 36.2, 33.3, 14.0.

LRMS (ESI) m/z: 457.1 [M+H]⁺. HRMS (ESI) m/z: calcd for C₂₆H₂₅N₄O₄, 457.1876 [M+H]⁺; found, 457.1799.

HPLC purity (method A): 98.6% (t_{R}=35.47 min).

### Example 6: 3-({5-[3-(Acryloylamino)phenyl]-6-phenylfuro[2,3-d]pyrimidin-4-yl}amino)propanoic Acid (6)

To a solution of 5 (164 mg, 0.36 mmol, 1 equiv) in THF (10 mL) at 0° C. was added 0.5 M LiOH_{(aq)} (2 mL, 1.02 mmol, 2.8 equiv); then, the reaction mixture was stirred at room temperature. After stirring for 3 h, the reaction mixture was concentrated in vacuo and purified by CombiFlash automated flash chromatography (5% methanol in dichloromethane) to yield the title compound (142 mg, 0.33 mmol, 92%) as a light-yellow solid.

¹H NMR (600 MHz, DMSO-d₆) δ 10.40-10.31 (m, 1H), 8.36 (s, 1H), 7.87 (ddd, J=8.4, 2.4, 1.2 Hz, 1H), 7.76-7.71 (m, 1H), 7.51 (t, J=8.4 Hz, 1H), 7.47-7.43 (m, 2H), 7.39-7.30 (m, 3H) 7.19-7.15 (m, 1H), 6.43 (dd, J=16.8, 10.2 Hz, 1H), 6.26 (dd, J=16.8, 2.1 Hz, 1H), 5.76 (dd, J=10.2, 2.1 Hz, 1H), 5.69-5.63 (m, 1H), 3.60 (td, J=6.6, 6.0 Hz, 2H), 2.47-2.41 (m, 2H). ¹³C NMR (150 MHz, DMSO-d₆) δ 173.3, 164.6, 163.4, 156.9, 154.0, 145.7, 140.0, 131.7, 131.6, 130.3, 129.0, 128.8, 128.7, 127.2, 126.1, 124.4, 120.1, 119.7, 115.2, 102.2, 36.4, 33.4.

LRMS (ESI) m/z: 429.1 [M+H]⁺. HRMS (ESI) m/z: calcd for C₂₄H₂₁N₄O₄, 429.1563 [M+H]⁺; found, 429.1560.

HPLC purity (method A): 98.8% (t_{R}=26.74 min).

### Example 7: N-(3-{4-[(2-Aminoethyl)amino]-6-phenylfuro[2,3-d]pyrimidin-5-yl}phenyl)prop-2-enamide (7)

Compound tert-Butyl [2-({5-[3-(acryloylamino)phenyl]-6-phenylfuro[2,3-d]pyrimidin-4-yl}amino)ethyl]carbamate (22′) was prepared following the procedure described in Li et al., J. Med. Chem. 66, 2566-88 (2023), Supporting Information.

To a solution of 22′ (215 mg, 0.43 mmol, 1 equiv) in dichloromethane (5 mL) was added trifluoroacetic acid (1 mL); then, the reaction mixture was stirred at room temperature. After stirring for 1 h, the reaction mixture was concentrated in vacuo and purified by flash column chromatography (3-20% methanol in dichloromethane) to yield the title compound (165 mg, 0.41 mmol, 96%) as a white solid.

¹H NMR (600 MHz, DMSO-d₆) δ 10.48 (s, 1H), 8.40 (s, 1H), 7.90 (s, 1H), 7.81-7.68 (m, 3H), 7.50 (td, J=7.8, 1.8 Hz, 1H), 7.44 (d, J=7.8 Hz, 2H), 7.40-7.31 (m, 3H), 7.19 (dd, J=7.8, 1.2 Hz, 1H), 6.47 (dd, J=17.1, 10.2 Hz, 1H), 6.29 (dd, J=17.1, 2.1 Hz, 1H), 5.88 (t, J=6.0 Hz, 1H), 5.79 (dd, J=10.2, 2.1 Hz, 1H), 3.66 (td, J=6.3, 6.0 Hz, 2H), 3.02 (td, J=6.3, 1.8 Hz, 2H). ¹³C NMR (150 MHz, DMSO-d₆) δ 164.7, 163.6, 157.3, 153.7, 146.1, 139.8, 131.6, 131.5, 130.2, 128.92, 128.90, 128.8, 127.5, 126.4, 124.9, 120.6, 119.9, 115.3, 102.5, 38.6, 38.2.

LRMS (ESI) m/z: 400.1 [M+H]⁺. HRMS (ESI) m/z: calcd for C₂₃H₂₂N₅O₂, 400.1774 [M+H]⁺; found, 400.1779.

HPLC purity (method A): 96.9% (t_{R}=19.93 min).

### Example 8: N-(3-(4-{[2-(Dimethylamino)ethyl]amino}-6-phenylfuro[2,3-d]pyrimidin-5-yl)phenyl)prop-2-enamide (8)

Compound 70i (see Scheme I) was prepared following the procedure described in Li et al., J. Med. Chem. 66, 2566-88 (2023), Supporting Information.

To a solution of 70i (324 mg, 0.87 mmol, 1 equiv) in dichloromethane (5 mL) were added triethylamine (160 μL, 1.15 mmol, 1.3 equiv) and acryloyl chloride (85 L, 1.05 mmol, 1.2 equiv); then, the reaction mixture was stirred at room temperature. After stirring for 4 h, the reaction mixture was concentrated in vacuo and purified by flash column chromatography (1-5% methanol in dichloromethane) to yield the title compound (185 mg, 0.43 mmol, 50%) as a pale orange solid.

¹H NMR (600 MHz, DMSO-d₆) δ 10.62 (s, 1H), 8.39 (s, 1H), 7.93 (dd, J=2.4, 1.8 Hz, 1H), 7.85 (d, J=7.8 Hz, 1H), 7.49 (t, J=8.4 Hz, 1H), 7.45 (d, J=7.2 Hz, 2H), 7.39-7.31 (m, 3H), 7.18 (ddd, J=7.8, 1.8, 1.2 Hz, 1H), 6.53 (dd, J=17.4, 10.2 Hz, 1H), 6.27 (dd, J=17.4, 2.1 Hz, 1H), 5.89 (t, J=6.0 Hz, 1H), 5.77 (dd, J=10.2, 2.1 Hz, 1H), 3.73 (s, 2H), 3.11 (s, 2H), 2.64 (s, 6H). ¹³C NMR (150 MHz, DMSO-d₆) δ 164.7, 163.5, 157.1, 153.8, 146.0, 140.1, 131.7, 131.4, 130.2, 128.9, 128.82, 128.81, 127.2, 126.2, 124.8, 120.2, 119.7, 115.3, 102.5, 55.8, 42.8, 40.0, 36.2.

LRMS (ESI) m/z: 428.2 [M+H]⁺. HRMS (ESI) m/z: calcd for C₂₅H₂₆N₅O₂, 428.2087 [M+H]⁺; found, 428.2089.

HPLC purity (method A): 92.7% (t_{R}=21.41 min).

### Example 9: N-(3-{4-[(2-Methoxyethyl)amino]-6-phenylfuro[2,3-d]pyrimidin-5-yl}phenyl)prop-2-enamide (9)

Compound 70e (see Scheme I) was prepared following the procedure described in Li et al., J. Med. Chem. 66, 2566-88 (2023), Supporting Information.

To a solution of 70e (150 mg, 0.42 mmol, 1 equiv) in dichloromethane (5 mL) at 0° C. were added triethylamine (70 μL, 0.5 mmol, 1.2 equiv) and a solution of acryloyl chloride (40 μL, 0.49 mmol, 1.2 equiv) in dichloromethane (2 mL); then, the reaction mixture was stirred at room temperature. After stirring for 1 h, the reaction mixture was concentrated in vacuo and purified by CombiFlash automated flash chromatography (2% methanol in dichloromethane) to yield the title compound (148 mg, 0.36 mmol, 86%) as a light-yellow solid.

¹H NMR (600 MHz, DMSO-d₆) δ 10.36 (s, 1H), 8.35 (s, 1H), 7.87 (ddd, J=8.4, 2.4, 1.2 Hz, 1H), 7.83 (dd, J=2.4, 2.4 Hz, 1H), 7.54 (t, J=7.8 Hz, 1H), 7.49 (d, J=8.4 Hz, 2H), 7.39-7.30 (m, 3H), 7.20 (ddd, J=7.8, 2.4, 1.2 Hz, 1H), 6.44 (dd, J=16.8, 10.2 Hz, 1H), 6.26 (dd, J=16.8, 1.8 Hz, 1H), 5.78 (dd, J=10.2, 1.8 Hz, 1H), 5.35 (t, J=5.4 Hz, 1H), 3.52 (td, J=5.4, 5.4 Hz, 2H), 3.10 (s, 3H). ¹³C NMR (150 MHz, DMSO-d₆) δ 164.5, 163.4, 157.0, 154.1, 145.6, 140.2, 131.9, 131.6, 130.4, 129.0, 128.9, 128.8, 127.4, 126.0, 124.4, 119.8, 119.6, 115.1, 102.3, 69.8, 57.9, 39.9.

LRMS (ESI) m/z: 415.1 [M+H]⁺. HRMS (ESI) m/z: calcd for C₂₄H₂₂N₄NaO₃, 437.1590 [M+Na]⁺; found, 437.1593.

HPLC purity (method A): 100% (t_{R}=31.83 min).

### Example 10: N-(3-{4-[(2-Phenoxyethyl)amino]-6-phenylfuro[2,3-d]pyrimidin-5-yl}phenyl)prop-2-enamide (10)

Compound 70f (see Scheme I) was prepared following the procedure described in Li et al., J. Med. Chem. 66, 2566-88 (2023), Supporting Information.

To a solution of 70f (34 mg, 0.08 mmol, 1 equiv) in dichloromethane (15 mL) at 0° C. were added triethylamine (17 μL, 0.12 mmol, 2.2 equiv) and a solution of acryloyl chloride (8 μL, 0.1 mmol, 1.2 equiv) in dichloromethane (5 mL); then, the reaction mixture was stirred at room temperature. After stirring for 1 h, the reaction mixture was concentrated in vacuo and purified by flash column chromatography (100% dichloromethane) to yield the title compound (16 mg, 0.03 mmol, 42%) as a yellow solid.

¹H NMR (600 MHz, DMSO-d₆) δ 10.31 (s, 1H), 8.39 (s, 1H), 7.85-7.81 (m, 2H), 7.51-7.44 (m, 3H), 7.39-7.30 (m, 3H), 7.23 (dd, J=8.7, 7.5 Hz, 2H), 7.19 (ddd, J=7.2, 1.8, 1.2 Hz, 1H), 6.91 (td, J=7.5, 1.2 Hz, 1H), 6.80 (dd, J=8.7, 1.2 Hz, 1H), 6.39 (dd, J=16.8, 10.2 Hz, 1H), 6.25 (dd, J=16.8, 1.8 Hz, 1H), 5.76 (dd, J=10.2, 1.8 Hz, 1H), 5.53 (t, J=6.0 Hz, 1H), 4.03 (t, J=6.0 Hz, 2H), 3.79 (td, J=6.0, 5.4 Hz, 2H). ¹³C NMR (150 MHz, DMSO-d₆) δ 164.5, 163.4, 158.0, 157.0, 154.0, 145.8, 140.1, 131.8, 131.6, 130.3, 129.4, 128.9, 128.83, 128.77, 127,2, 126.0, 124.4, 120.7, 119.9, 119.7, 115.1, 114.4, 102.4, 65.6, 39.5.

LRMS (ESI) m/z: 477.2 [M+H]⁺. HRMS (ESI) m/z: calcd for C₂₉H₂₅N₄O₃, 477.1927 [M+H]⁺; found, 477.1923.

HPLC purity (method A): 91.5% (t_{R}=40.64 min).

### Example 11: N-(3-{4-[(2-Hydroxyethyl)amino]furo[2,3-d]pyrimidin-5-yl}phenyl)prop-2-enamide (11)

Compound 73a (see Scheme II) was prepared following the procedure described in Li et al., J. Med. Chem. 66, 2566-88 (2023), Supporting Information.

To a solution of 73a (59 mg, 0.22 mmol, 1 equiv) in THF (10 mL) at 0° C. were added triethylamine (36 μL, 0.26 mmol, 1.2 equiv) and a solution of acryloyl chloride (19 μL, 0.24 mmol, 1.1 equiv) in THF (5 mL); then, the reaction mixture was stirred at room temperature. After stirring for 3 h, the reaction mixture was concentrated in vacuo and purified by flash column chromatography (5% methanol in dichloromethane) to yield the title compound (27 mg, 0.08 mmol, 38%) as a white solid.

¹H NMR (600 MHz, DMSO-d₆) δ 10.33 (s, 1H), 8.34 (s, 1H), 8.00 (s, 1H), 7.91 (dd, J=2.4, 1.8 Hz, 1H), 7.66 (ddd, J=8.4, 2.4, 1.2 Hz, 1H), 7.48 (dd, J=8.4, 7.2 Hz, 1H), 7.26 (ddd, J=7.2, 1.8, 1.2 Hz, 1H), 6.46 (dd, J=17.1, 10.2 Hz, 1H), 6.29 (dd, J=17.1, 1.5 Hz, 1H), 6.05 (t, J=5.4 Hz, 1H), 5.79 (dd, J=10.2, 1.5 Hz, 1H), 4.70 (td, J=5.4, 1.2 Hz, 1H), 3.60-3.50 (m, 4H). ¹³C NMR (150 MHz, DMSO-d₆) δ 166.4, 163.5, 157,5, 153.8, 139.7, 138.4, 131.7, 131.2, 129.9, 127.3, 123.3, 120.5, 119.2, 119.1, 99.0, 59.2, 43.0.

LRMS (ESI) m/z: 325.1 [M+H]⁺. HRMS (ESI) m/z: calcd for C₁₇H₁₇N₄O₃, 325.1301 [M+H]⁺; found, 325.1303.

HPLC purity (method A): 96.0% (t_{R}=18.46 min).

### Example 12: N-(3-{4-[(3-Hydroxypropyl)amino]-6-phenylfuro[2,3-d]pyrimidin-5-yl}phenyl)prop-2-enamide (12)

Compound 70d (see Scheme I) was prepared following the procedure described in Li et al., J. Med. Chem. 66, 2566-88 (2023), Supporting Information.

To a solution of 70d (31 mg, 0.09 mmol, 1 equiv) in dichloromethane (10 mL) at 0° C. were added DIPEA (36 μL, 0.17 mmol, 2 equiv) and a solution of acryloyl chloride (8 μL, 0.1 mmol, 1.2 equiv) in dichloromethane (1 mL); then, the reaction mixture was stirred at room temperature. After stirring for 2 h, the reaction mixture was concentrated in vacuo and purified by thin-layer chromatography (5% methanol in dichloromethane) to yield the title compound (25 mg, 0.06 mmol, 70%) as a white solid.

¹H NMR (600 MHz, DMSO-d₆) δ 10.33 (s, 1H), 8.34 (s, 1H), 7.84 (dd, J=8.4, 1.8 Hz, 1H), 7.79 (dd, J=1.8, 1.8 Hz, 1H), 7.53 (t, J=7.8 Hz, 1H), 7.45 (d, J=7.8 Hz, 2H), 7.38-7.29 (m, 3H), 7.21 (d, J=7.8 Hz, 1H), 6.43 (dd, J=17.4, 10.2 Hz, 1H), 6.26 (dd, J=17.4, 2.1 Hz, 1H), 5.77 (dd, J=10.2, 2.1 Hz, 1H), 5.46 (t, J=6.0 Hz, 1H), 4.39 (t, J=5.7, 1H), 3.46 (td, J=6.6, 5.7 Hz, 2H), 3.37-3.33 (m, 2H), 1.56 (quin, J=6.6 Hz, 2H).

¹³C NMR (150 MHz, DMSO-d₆) δ 164.5, 163.4, 157.1, 154.0, 145.5, 140.0, 131.8, 131.6, 130.3, 129.0, 128.8, 128.7, 127.3, 126.0, 124.5, 120.1, 119.7, 115.2, 102.1, 58.6, 38.2, 31.6.

LRMS (ESI) m/z: 415.1 [M+H]⁺. HRMS (ESI) m/z: calcd for C₂₄H₂₃N₄O₃, 415.1770 [M+H]⁺; found, 415.1769.

HPLC purity (method A): 99.6% (t_{R}=26.85 min).

### Example 13: N-(3-{4-[(2-Hydroxyethyl)amino)-6-[4-(4-methylpiperazin-1-yl)phenyl]furo[2,3-d]pyrimidin-5-yl}phenyl)prop-2-enamide (13)

Compound 73e (see Scheme II) was prepared following the procedure described in Li et al., J. Med. Chem. 66, 2566-88 (2023), Supporting Information.

To a solution of 73e (140 mg, 0.25 mmol, 1 equiv) in dichloromethane (7.5 mL) were added triethylamine (40 μL, 0.29 mmol, 1.1 equiv) and a solution of acryloyl chloride (20 μL, 0.25 mmol, 1 equiv) in dichloromethane (7.5 mL) dropwise at 0° C., then the reaction mixture was stirred at room temperature. After stirring for 3 h, to the reaction mixture was added H₂O (10 mL) and extracted with dichloromethane (10 mL×3). The organic layers were combined, washed with brine (10 mL), dried over MgSO₄, concentrated in vacuo, and purified by flash column chromatography (2.5% methanol in dichloromethane with 0.3% NH₄OH) to yield the TBS-protected compound 35′. To the TBS-protected compound 35′ in dichloromethane (4 mL) was added trifluoroacetic acid (0.4 mL); then, the reaction mixture was stirred at room temperature. After stirring for 8 h, the reaction mixture was concentrated in vacuo, neutralized with 10% NH₄OH in methanol, then concentrated in vacuo again and purified by flash column chromatography (5% methanol in dichloromethane with 0.5% NH₄OH) to yield the title compound (43 mg, 0.09 mmol, 34%) as a white solid.

¹H NMR (600 MHz, DMSO-d₆) δ 10.30 (s, 1H), 8.29 (s, 1H), 7.86 (dd, J=8.4, 2.4 Hz, 1H), 7.76 (dd, J=2.4, 1.8 Hz, 1H), 7.51 (dd, J=8.4, 7.5 Hz, 1H), 7.30 (d, J=9.0 Hz, 2H), 7.20 (ddd, J=7.5, 1.8, 1.2 Hz, 1H), 6.88 (d, J=9.0 Hz, 2H), 6.43 (dd, J=17.1, 10.2 Hz, 1H), 6.26 (dd, J=17.1, 1.8 Hz, 1H), 5.77 (dd, J=10.2, 1.8 Hz, 1H), 5.41 (t, J=5.4 Hz, 1H), 4.63 (s, 1H), 3.50-3.40 (m, 4H), 3.17 (dd, J=4.8, 4.8 Hz, 4H), 2.39 (dd, J=4.8, 4.8 Hz, 4H), 2.19 (s, 3H). ¹³C NMR (150 MHz, DMSO-d₆) δ 164.2, 163.4, 156.8, 153.2, 150.7, 146.5, 140.0, 132.3, 131.7, 130.2, 127.2, 127.0, 124.5, 120.2, 119.4, 118.5, 114.5, 112.2, 102.3, 59.1, 54.3, 47.0, 45.7, 42.8.

LRMS (ESI) m/z: 499.2 [M+H]⁺. HRMS (ESI) m/z: calcd for C₂₈H₃₁N₆O₃, 499.2458 [M+H]⁺; found, 499.2460.

HPLC purity (method A): 99.2% (t_{R}=15.46 min).

### Example 14: N-(3-{6-[4-(Dimethylamino)phenyl]-4-[(2-hydroxyethyl)amino]-furo[2,3-d]pyrimidin-5-yl}phenyl)prop-2-enamide (14)

Compound 73c (see Scheme II) was prepared following the procedure described in Li et al., J. Med. Chem. 66, 2566-88 (2023), Supporting Information.

To a solution of 73c (155 mg, 0.31 mmol, 1 equiv) in dichloromethane (15 mL) were added triethylamine (60 μL, 0.43 mmol, 1.4 equiv) and a solution of acryloyl chloride (30 μL, 0.37 mmol, 1.2 equiv) in dichloromethane (15 mL) dropwise at 0° C., then the reaction mixture was stirred at room temperature. After stirring for 6 h, to the reaction mixture was added H₂O (10 mL) and extracted with dichloromethane (10 mL×3). The organic layers were combined, washed with brine (10 mL), dried over MgSO₄, concentrated in vacuo, and purified by thin-layer chromatography (2% methanol in dichloromethane) to yield the TBS-protected compound 33′. To the TBS-protected compound 33′ in dichloromethane (8 mL) was added trifluoroacetic acid (0.4 mL). The reaction mixture was stirred at room temperature for 6 h and then concentrated in vacuo. The obtained residue was neutralized with 10% NH₄OH in methanol, then concentrated in vacuo again and purified by flash column chromatography (3% methanol in dichloromethane) to yield the title compound (90 mg, 0.2 mmol, 66%) as a light-yellow solid.

¹H NMR (600 MHz, DMSO-d₆) δ 10.30 (s, 1H), 8.28 (s, 1H), 7.85 (d, J=8.4 Hz, 1H), 7.76 (dd, J=1.8, 1.8 Hz, 1H), 7.51 (dd, J=8.4, 7.8 Hz, 1H), 7.29 (d, J=9.0 Hz, 2H), 7.20 (ddd, J=7.8, 1.8, 1.2 Hz, 1H), 6.65 (d, J=9.0 Hz, 2H), 6.43 (dd, J=17.1, 10.2 Hz, 1H), 6.26 (dd, J=17.1, 2.1 Hz, 1H), 5.77 (dd, J=10.2, 2.1 Hz, 1H), 5.39 (t, J=5.4 Hz, 1H), 4.62 (t, J=4.8 Hz, 1H), 3.50-3.40 (m, 4H), 2.90 (s, 6H). ¹³C NMR (150 MHz, DMSO-d₆) δ 164.1, 163.4, 156.7, 152.9, 150.2, 147.0, 139.9, 132.5, 131.7, 130.2, 127.2, 127.1, 124.6, 120.3, 119.3, 116.1, 111.8, 102.4, 59.2, 42.8, 39.9.

LRMS (ESI) m/z: 324.1 [M+H]⁺. HRMS (ESI) m/z: calcd for C₂₅H₂₅N₅NaO₃, 466.4968 [M+Na]⁺; found, 466.1994.

HPLC purity (method A): 97.5% (t_{R}=27.85 min).

### Example 15: N-(3-{4-[(2-Hydroxyethyl)amino]-6-[4-(morpholin-4-yl)phenyl]furo[2,3-d]pyrimidin-5-yl}phenyl)prop-2-enamide (15)

Compound 73d (see Scheme II) was prepared following the procedure described in Li et al., J. Med. Chem. 66, 2566-88 (2023), Supporting Information.

To a solution of 73d (94 mg, 0.17 mmol, 1 equiv) in dichloromethane (8 mL) were added triethylamine (34 μL, 0.25 mmol, 1.4 equiv) and a solution of acryloyl chloride (17 μL, 0.21 mmol, 1.2 equiv) in dichloromethane (8 mL) dropwise at 0° C., then the reaction mixture was stirred at room temperature. After stirring for 6 h, to the reaction mixture was added H₂O (10 mL) and extracted with dichloromethane (10 mL×3). The organic layers were combined, washed with brine (10 mL), dried over MgSO₄, and concentrated in vacuo to yield the TBS-protected compound 34′. To the TBS-protected compound 34′ in dichloromethane (4 mL) was added trifluoroacetic acid (0.2 mL); then, the reaction mixture was stirred at room temperature. After stirring for 12 h, the reaction mixture was concentrated in vacuo, neutralized with 10% NH₄OH in methanol, then concentrated in vacuo again and purified by flash column chromatography (3% methanol in dichloromethane) to yield the title compound (40 mg, 0.08 mmol, 48%) as a light-yellow solid.

¹H NMR (600 MHz, DMSO-d₆) δ 10.32 (s, 1H), 8.29 (s, 1H), 7.86 (ddd, J=8.1, 2.4, 1.2 Hz, 1H), 7.77 (dd, J=2.4, 1.8 Hz, 1H), 7.51 (dd, J=8.1, 7.8 Hz, 1H), 7.32 (d, J=9.0 Hz, 2H), 7.20 (ddd, J=7.8, 1.8, 1.2 Hz, 1H), 6.90 (d, J=9.0 Hz, 2H), 6.44 (dd, J=16.8, 10.2 Hz, 1H), 6.26 (dd, J=16.8, 2.1 Hz, 1H), 5.77 (dd, J=10.2, 2.1 Hz, 1H), 5.42 (t, J=5.4 Hz, 1H), 4.63 (t, J=4.8 Hz, 1H), 3.74-3.66 (m, 4H), 3.49-3.40 (m, 4H), 3.18-3.10 (m, 4H). ¹³C NMR (150 MHz, DMSO-d₆) δ 164.2, 163.4, 156.8, 153.2, 150.8, 146.4, 140.0, 132.3, 131.7, 130.2, 127.2, 127.0, 124.5, 120.2, 119.4, 119.0, 114.3, 112.4, 102.3, 65.9, 59.1, 47.3, 42.8.

LRMS (ESI) m/z: 486.2 [M+H]⁺. HRMS (ESI) m/z: calcd for C₂₇H₂₇N₅NaO₄, 508.1961 [M+Na]⁺; found, 508.1961.

HPLC purity (method A): 97.6% (t_{R}=24.57 min).

### Example 16: N-{3-[4-(Cyclopropylamino)-6-phenylfuro[2,3-d]pyrimidin-5-yl]phenyl}prop-2-enamide (16)

Compound 78d (see Scheme III) was prepared following the procedure described in Li et al., J. Med. Chem. 66, 2566-88 (2023), Supporting Information.

To a solution of 78d (30 mg, 0.09 mmol, 1 equiv) in dichloromethane (3 mL) were added DIPEA (3 μL, 0.02 mmol, 20 mol % mol %), acrylic acid (8 μL, 0.12 mmol, 1.3 equiv), and EDCI (20 mg, 0.1 mmol, 1.2 equiv); then, the reaction mixture was stirred at room temperature. After stirring for 16 h, to the reaction mixture was added ethyl acetate (5 mL) and washed with NaHCO_{3(aq)} (10 mL) and brine (10 mL). The combined organic layers were dried over MgSO₄, concentrated in vacuo, and purified by flash column chromatography (33-66% ethyl acetate in hexane) to yield the title compound (27 mg, 0.07 mmol, 78%) as a white solid.

¹H NMR (600 MHz, DMSO-d₆) δ 10.35 (s, 1H), 8.42 (s, 1H), 7.84 (dd, J=2.4, 1.8 Hz, 1H), 7.79 (ddd, J=8.4, 2.4, 1.2 Hz, 1H), 7.53 (t, J=7.8 Hz, 1H), 7.48 (d, J=10.2 Hz, 2H), 7.39-7.31 (m, 3H), 7.20 (ddd, J=7.8, 1.8, 1.2 Hz, 1H), 6.43 (dd, J=17.1, 10.2 Hz, 1H), 6.27 (dd, J=17.1, 2.1 Hz, 1H), 5.78 (dd, J=10.2, 2.1 Hz, 1H), 5.26 (d, J=3.3 Hz, 1H), 2.79 (dset, J=3.6, 3.3 Hz, 1H), 0.73-0.67 (m, 2H), 0.34-0.29 (m, 2H). ¹³C NMR (150 MHz, DMSO-d₆) δ 164.4, 163.5, 158.1, 154.0, 145.8, 140.0, 131.9, 131.6, 130.3, 128.9, 128.84, 128.83, 127.4, 126.1, 124.6, 120.0, 119.7, 115.0, 102.4, 23.9, 7.0.

LRMS (ESI) m/z: 397.1 [M+H]⁺. HRMS (ESI) m/z: calcd for C₂₄H₂₁N₄O₂, 397.1665 [M+H]⁺; found, 397.1663.

HPLC purity (method B): 93.8% (t_{R}=22.85 min).

### Example 17: N-{3-[6-Phenyl-4-(propan-2-ylamino)furo[2,3-d]pyrimidin-5-yl]phenyl}prop-2-enamide (17)

Compound 78c (see Scheme III) was prepared following the procedure described in Li et al., J. Med. Chem. 66, 2566-88 (2023), Supporting Information.

To a solution of 78c (30 mg, 0.09 mmol, 1 equiv) in dichloromethane (3 mL) were added DIPEA (3 μL, 0.02 mmol, 20 mol %), acrylic acid (8 μL, 0.12 mmol, 1.4 equiv), and EDCI (20 mg, 0.1 mmol, 1.2 equiv); then, the reaction mixture was stirred at room temperature. After stirring for 16 h, to the reaction mixture was added ethyl acetate (5 mL) and washed with NaHCO_{3(aq)} (10 mL) and brine (10 mL). The combined organic layers were dried over MgSO₄, concentrated in vacuo, and purified by flash column chromatography (17-50% ethyl acetate in hexane) to yield the title compound (27 mg, 0.07 mmol, 58%) as a white solid.

¹H NMR (600 MHz, DMSO-d₆) δ 10.38 (s, 1H), 8.36 (s, 1H), 7.93 (dd, J=2.1, 1.8 Hz, 1H), 7.79 (ddd, J=7.8, 2.1, 1.2 Hz, 1H), 7.56 (t, J=8.4 Hz, 1H), 7.50 (d, J=7.8 Hz, 2H), 7.40-7.31 (m, 3H), 7.24 (ddd, J=7.8, 1.8, 1.2 Hz, 1H), 6.43 (dd, J=16.8, 10.2 Hz, 1H), 6.27 (dd, J=16.8, 2.1 Hz, 1H), 5.78 (dd, J=10.2, 2.1 Hz, 1H), 4.85 (d, J=7.2 Hz, 1H), 4.18 (dset, J=7.2, 6.3 Hz, 1H), 1.03 (d, J=6.3 Hz, 6H).

¹³C NMR (150 MHz, DMSO-d₆) δ 164.5, 163.5, 156.4, 154.1, 145.5, 140.1, 132.0, 131.6, 130.3, 128.94, 128.86, 128.8, 127.4, 126.0, 124.5, 119.9, 119.7, 115.0, 102.0, 42.3, 22.0.

LRMS (ESI) m/z: 399.1 [M+H]⁺. HRMS (ESI) m/z: calcd for C₂₄H₂₃N₄O₂, 399.1821 [M+H]⁺; found, 399.1814.

HPLC purity (method B): 99.1% (t_{R}=25.66 min).

### Example 18: N-{3-[4-(Dimethylamino)-6-phenylfuro[2,3-d]pyrimidin-5-yl]phenyl}prop-2-enamide (18)

Compound 78b (see Scheme III) was prepared following the procedure described in Li et al., J. Med. Chem. 66, 2566-88 (2023), Supporting Information.

To a solution of 78b (40 mg, 0.12 mmol, 1 equiv) in dichloromethane (3 mL) were added DIPEA (4 μL, 0.02 mmol, 18 mol %), acrylic acid (11 μL, 0.16 mmol, 1.3 equiv), and EDCI (28 mg, 0.15 mmol, 1.2 equiv); then, the reaction mixture was stirred at room temperature. After stirring for 16 h, to the reaction mixture was added ethyl acetate (5 mL) and washed with NaHCO_{3(aq)} (10 mL) and brine (10 mL). The combined organic layers were dried over MgSO₄, concentrated in vacuo, and purified by flash column chromatography (17-66% ethyl acetate in hexane) to yield the title compound (27 mg, 0.07 mmol, 58%) as a light-yellow solid.

¹H NMR (600 MHz, DMSO-d₆) δ 10.29 (s, 1H), 8.36 (s, 1H), 7.85 (ddd, J=8.4, 2.1, 1.2 Hz, 1H), 7.69 (dd, J=2.1, 1.8 Hz, 1H), 7.47 (t, J=7.8 Hz, 1H), 7.41 (d, J=7.2 Hz, 2H), 7.37-7.29 (m, 3H), 7.14 (ddd, J=7.2, 1.8, 1.2 Hz, 1H), 6.42 (dd, J=16.8, 10.2 Hz, 1H), 6.24 (dd, J=16.8, 1.8 Hz, 1H), 5.76 (dd, J=10.2, 1.8 Hz, 1H), 2.71 (s, 6H). ¹³C NMR (150 MHz, DMSO-d₆) δ 166.3, 163.3, 159.8, 152.3, 145.6, 139.7, 134.2, 131.7, 129.8, 129.3, 128.7, 128.6, 127.2, 126.5, 125.4, 120.7, 119.2, 116.1, 103.5, 40.2.

LRMS (ESI) m/z: 385.1 [M+H]⁺. HRMS (ESI) m/z: calcd for C₂₃H₂₁N₄O₂, 385.1665 [M+H]⁺; found, 385.1677.

HPLC purity (method B): 93.1% (t_{R}=23.09 min).

### Example 19: N-{3-[4-(Cyclopentylamino)-6-phenylfuro[2,3-d]pyrimidin-5-yl]phenyl}prop-2-enamide (19)

Compound 78f (see Scheme III) was prepared following the procedure described in Li et al., J. Med. Chem. 66, 2566-88 (2023), Supporting Information.

To a solution of 78f (37 mg, 0.1 mmol, 1 equiv) in dichloromethane (3 mL) were added DIPEA (3 μL, 0.02 mmol, 17 mol %), acrylic acid (9 μL, 0.14 mmol, 1.4 equiv), and EDCI (23 mg, 0.12 mmol, 1.2 equiv); then, the reaction mixture was stirred at room temperature. After stirring for 16 h, to the reaction mixture was added ethyl acetate (5 mL) and washed with NaHCO_{3(aq)} (10 mL) and brine (10 mL). The combined organic layers were dried over MgSO₄, concentrated in vacuo, and washed with cold methanol (5 mL) to yield the title compound (20 mg, 0.05 mmol, 47%) as a white solid.

¹H NMR (600 MHz, DMSO-d₆) δ 10.39 (s, 1H), 8.36 (s, 1H), 7.93 (dd, J=2.1, 1.8 Hz, 1H), 7.80 (ddd, J=8.4, 2.1, 1.2 Hz, 1H), 7.56 (t, J=7.8 Hz, 1H), 7.52 (d, J=7.8 Hz, 2H), 7.40-7.32 (m, 3H), 7.25 (ddd, J=7.8, 1.8, 1.2 Hz, 1H), 6.43 (dd, J=16.8, 10.2 Hz, 1H), 6.26 (dd, J=16.8, 1.8 Hz, 1H), 5.78 (dd, J=10.2, 1.8 Hz, 1H), 4.89 (d, J=7.2 Hz, 1H), 4.39-4.30 (m, 1H), 1.87-1.77 (m, 2H), 1.54-1.44 (m, 2H), 1.43-1.33 (m, 2H), 1.26-1.17 (m, 2H). ¹³C NMR (150 MHz, DMSO-d₆) δ 164.4, 163.5, 156.6, 154.2, 145.4, 140.2, 132.2, 131.5, 130.4, 128.94, 128.89, 128.79, 127.5, 125.9, 124.5, 119.8, 119.6, 115.0, 102.3, 52.0, 32.5, 22.7.

LRMS (ESI) m/z: 425.2 [M+H]⁺. HRMS (ESI) m/z: calcd for C₂₆H₂₅N₄O₂, 425.1978 [M+H]⁺; found, 425.1920.

HPLC purity (method B): 97.8% (t_{R}=27.60 min).

### Example 20: N-{3-[4-(Methylamino)-6-phenylfuro[2,3-d]pyrimidin-5-yl]phenyl}prop-2-enamide (20)

Compound 78a (see Scheme III) was prepared following the procedure described in Li et al., J. Med. Chem. 66, 2566-88 (2023), Supporting Information.

To a solution of 78a (40 mg, 0.13 mmol, 1 equiv) in dichloromethane (3 mL) were added DIPEA (4 μL, 0.02 mmol, 18 mol %), acrylic acid (11 μL, 0.16 mmol, 1.3 equiv), and EDCI (29 mg, 0.15 mmol, 1.2 equiv); then, the reaction mixture was stirred at room temperature. After stirring for 16 h, to the reaction mixture was added ethyl acetate (5 mL) and washed with NaHCO_{3 (aq)} (10 mL) and brine (10 mL). The combined organic layers were dried over MgSO₄, concentrated in vacuo and purified by flash column chromatography (33-66% ethyl acetate in hexane) to yield the title compound (32 mg, 0.09 mmol, 68%) as a white solid.

¹H NMR (600 MHz, DMSO-d₆) δ 10.34 (s, 1H), 8.36 (s, 1H), 7.85 (ddd, J=8.4, 2.1, 1.2 Hz, 1H), 7.75 (dd, J=2.1, 1.8 Hz, 1H), 7.52 (t, J=7.8 Hz, 1H), 7.43 (d, J=7.2 Hz, 2H), 7.38-7.29 (m, 3H), 7.17 (ddd, J=7.8, 1.8, 1.2 Hz, 1H), 6.44 (dd, J=17.1, 10.2 Hz, 1H), 6.27 (dd, J=17.1, 2.4 Hz, 1H), 5.77 (dd, J=10.2, 2.4 Hz, 1H), 5.54 (q, J=4.8 Hz, 1H), 2.89 (d, J=4.8 Hz, 3H).

¹³C NMR (150 MHz, DMSO-d₆) δ 164.5, 163.4, 157.6, 154.0, 145.5, 140.0, 131.8, 131.6, 130.3, 129.0, 128.8, 128.7, 127.3, 126.1, 124.7, 120.2, 119.8, 115.3, 102.1, 28.0.

LRMS (ESI) m/z: 371.1 [M+H]⁺. HRMS (ESI) m/z: calcd for C₂₂H₁₇N₄O₂, 369.1352 [M−H]+; found, 369.1351.

HPLC purity (method B): 97.0% (t_{R}=21.52 min).

### Example 21: N-{3-[4-(Cyclobutylamino)-6-phenylfuro[2,3-d]pyrimidin-5-yl]phenyl}prop-2-enamide (21)

Compound 78e (see Scheme III) was prepared following the procedure described in Li et al., J. Med. Chem. 66, 2566-88 (2023), Supporting Information.

To a solution of 78e (38 mg, 0.11 mmol, 1 equiv) in dichloromethane (3 mL) were added DIPEA (4 μL, 0.02 mmol, 22 mol %), acrylic acid (10 μL, 0.15 mmol, 1.4 equiv), and EDCI (27 mg, 0.14 mmol, 1.3 equiv); then, the reaction mixture was stirred at room temperature. After stirring for 16 h, to the reaction mixture was added ethyl acetate (5 mL) and washed with NaHCO_{3 (aq)} (10 mL) and brine (10 mL). The combined organic layers were dried over MgSO₄, concentrated in vacuo, and purified by flash column chromatography (17-33% ethyl acetate in hexane) to yield the title compound (31 mg, 0.08 mmol, 71%) as a white solid.

¹H NMR (600 MHz, DMSO-d₆) δ 10.40 (s, 1H), 8.34 (s, 1H), 7.93 (dd, J=2.1, 1.8 Hz, 1H), 7.80 (ddd, J=8.4, 2.1, 1.2 Hz, 1H), 7.57 (t, J=8.4 Hz, 1H), 7.50 (d, J=7.2 Hz, 2H), 7.40-7.32 (m, 3H), 7.25 (ddd, J=7.2, 1.8, 1.2 Hz, 1H), 6.44 (dd, J=17.1, 10.2 Hz, 1H), 6.27 (dd, J=17.1, 1.8 Hz, 1H), 5.78 (dd, J=10.2, 1.8 Hz, 1H), 5.26 (d, J=7.5 Hz, 1H), 4.50 (dquin, J=7.8, 7.5 Hz, 1H), 2.27-2.20 (m, 2H), 1.70-1.57 (m, 4H). ¹³C NMR (150 MHz, DMSO-d₆) δ 164.6, 163.5, 156.1, 154.0, 145.7, 140.0, 132.0, 131.6, 130.3, 128.9, 128.85, 128.81, 127.4, 126.1, 124.6, 120.1, 119.8, 115.0, 102.0, 45.5, 30.5, 14.6.

LRMS (ESI) m/z: 411.2 [M+H]⁺. HRMS (ESI) m/z: calcd for C₂₅H₂₃N₄O₂, 411.1821 [M+H]⁺; found, 411.1813.

HPLC purity (method B): 99.2% (t_{R}=26.92 min).

### Example 22: N-(3-(4-(Cyclohexylamino)-6-phenylfuro[2,3-d]pyrimidin-5-yl)phenyl)prop-2-enamide (22)

Compound 78 g (see Scheme III) was prepared following the procedure described in Li et al., J. Med. Chem. 66, 2566-88 (2023), Supporting Information.

To a solution of 78 g (66 mg, 0.17 mmol, 1 equiv) in dichloromethane (0.9 mL) were added DIPEA (6 μL, 0.03 mmol, 20 mol %), acrylic acid (14 μL, 0.2 mmol, 1.2 equiv), and EDCI (49 mg, 0.26 mmol, 1.5 equiv); then, the reaction mixture was stirred at room temperature. After stirring for 16 h, the reaction mixture was concentrated in vacuo and purified by flash column chromatography (22% ethyl acetate in hexane) to yield the title compound (43 mg, 0.1 mmol, 57%) as a white solid.

¹H NMR (600 MHz, DMSO-d₆) δ 10.39 (s, 1H), 8.34 (s, 1H), 7.95 (dd, J=2.1, 1.8 Hz, 1H), 7.80 (ddd, J=8.4, 2.1, 1.2 Hz, 1H), 7.57 (t, J=7.8 Hz, 1H), 7.51 (d, J=7.2 Hz, 2H), 7.40-7.31 (m, 3H), 7.26 (ddd, J=7.8, 1.8, 1.2 Hz, 1H), 6.44 (dd, J=16.8, 10.2 Hz, 1H), 6.26 (dd, J=16.8, 1.8 Hz, 1H), 5.78 (dd, J=10.2, 1.8 Hz, 1H), 4.93 (d, J=7.8 Hz, 1H), 4.04-3.95 (m, 1H), 1.78-1.69 (m, 2H), 1.45-1.20 (m, 4H), 1.19-1.03 (m, 2H).

¹³C NMR (150 MHz, DMSO-d₆) δ 164.5, 163.5, 156.3, 154.2, 145.5, 140.2, 132.1, 131.5, 130.4, 128.94, 128.87, 128.77, 127.4, 125.9, 124.5, 119.74, 119.66, 115.0, 102.1, 47.9, 31.3, 25.0, 23.1.

LRMS (ESI) m/z: 439.2 [M+H]⁺. HRMS (ESI) m/z: calcd for C₂₇H₂₆N₄NaO₂, 461.1953 [M+Na]⁺; found, 461.1951.

HPLC purity (method B): 99.5% (t_{R}=28.82 min).

### Example 23: N-{3-[4-(Cyclopentylamino)-6-[4-(morpholin-4-yl)phenyl]furo[2,3-d]pyrimidin-5-yl]phenyl}prop-2-enamide (23)

Compound 78i (see Scheme III) was prepared following the procedure described in Li et al., J. Med. Chem. 66, 2566-88 (2023), Supporting Information.

To a solution of 78i (84 mg, 0.18 mmol, 1 equiv) in dichloromethane (3 mL) were added DIPEA (6 μL, 0.03 mmol, 19 mol %), acrylic acid (16 μL, 0.24 mmol, 1.3 equiv), and EDCI (42 mg, 0.22 mmol, 1.2 equiv); then, the reaction mixture was stirred at room temperature. After stirring for 16 h, to the reaction mixture was added ethyl acetate (5 mL) and washed with NaHCO_{3(aq)} (10 mL) and brine (10 mL). The combined organic layers were dried over MgSO₄, concentrated in vacuo, and purified by flash column chromatography (25-66% ethyl acetate in hexane) to yield the title compound (45 mg, 0.09 mmol, 48%) as a yellow solid.

¹H NMR (600 MHz, DMSO-d₆) δ 10.37 (s, 1H), 8.30 (s, 1H), 7.89 (dd, J=2.1, 1.8 Hz, 1H), 7.79 (dd, J=7.8, 2.1 Hz, 1H), 7.55 (dd, J=7.8, 7.8 Hz, 1H), 7.37 (d, J=9.0 Hz, 2H), 7.24-7.21 (m, 1H), 6.92 (d, J=9.0 Hz, 2H), 6.43 (dd, J=16.8, 10.2 Hz, 1H), 6.27 (dd, J=16.8, 2.1 Hz, 1H), 5.78 (dd, J=10.2, 2.1 Hz, 1H), 4.83 (d, J=7.2 Hz, 1H), 4.33 (dquin, J=7.2, 4.8 Hz, 1H), 3.70 (dd, J=12.0, 4.2 Hz, 4H), 3.15 (dd, J=12.0, 3.6 Hz, 4H), 1.86-1.77 (m, 2H), 1.53-1.33 (m, 4H), 1.26-1.15 (m, 2H).

¹³C NMR (150 MHz, DMSO-d₆) δ 164.1, 163.4, 156.2, 153.4, 150.9, 146.2, 140.1, 132.6, 131.6, 130.3, 127.4, 126.9, 124.6, 119.9, 119.4, 119.0, 114.4, 112.2, 102.4, 65.9, 51.9, 47.3, 32.5, 22.7.

LRMS (ESI) m/z: 510.2 [M+H]⁺. HRMS (ESI) m/z: calcd for C₃₀H₃₁N₅NaO₃, 532.2325 [M+Na]⁺; found, 532.2318.

HPLC purity (method B): 99.7% (t_{R}=26.25 min).

### Example 24: N-{3-[4-(Cyclopentylamino)-6-[4-(4-methylpiperazin-1-yl)phenyl]furo[2,3-d]pyrimidin-5-yl]phenyl}prop-2-enamide (24)

Compound 78j (see Scheme III) was prepared following the procedure described in Li et al., J. Med. Chem. 66, 2566-88 (2023), Supporting Information.

To a solution of 78j (40 mg, 0.09 mmol, 1 equiv) in dichloromethane (5 mL) at 0° C. were added acrylic acid (9 μL, 0.13 mmol, 1.5 equiv) and EDCI (25 mg, 0.42 mmol, 1.5 equiv); then, the reaction mixture was stirred at room temperature. After stirring for 16 h, the reaction mixture was quenched with NaHCO_{3 (aq)} (10 mL) and extracted with dichloromethane (10 mL×3). The combined organic layers were dried over MgSO₄, concentrated in vacuo, and purified by flash column chromatography (5% methanol in dichloromethane with 0.1% NH₄OH) to yield the title compound (35 mg, 0.07 mmol, 78%) as a yellow solid.

¹H NMR (600 MHz, DMSO-d₆) δ 10.37 (s, 1H), 8.30 (s, 1H), 7.89 (dd, J=2.1, 1.8 Hz, 1H), 7.79 (dd, J=8.1, 2.1, 1.2 Hz, 1H), 7.54 (dd, J=8.1, 7.5 Hz, 1H), 7.35 (d, J=9.0 Hz, 2H), 7.22 (ddd, J=7.5, 1.8, 1.2 Hz 1H), 6.90 (d, J=9.0 Hz, 2H), 6.44 (dd, J=16.8, 10.2 Hz, 1H), 6.27 (dd, J=16.8, 2.1 Hz, 1H), 5.78 (dd, J=10.2, 2.1 Hz, 1H), 4.82 (d, J=6.9 Hz, 1H), 4.33 (dquin, J=6.9, 5.4 Hz, 1H), 3.18 (dd, J=5.4, 5.1 Hz, 4H), 2.40 (dd, J=5.1, 4.8 Hz, 4H), 2.19 (s, 3H), 1.86-1.77 (m, 2H), 1.52-1.32 (m, 4H), 1.25-1.16 (m, 2H).

¹³C NMR (150 MHz, DMSO-d₆) δ 164.1, 163.4, 156.2, 153.3, 150.8, 146.3, 140.1, 132.7, 131.6, 130.2, 127.4, 126.9, 124.6, 119.9, 119.4, 118.5, 114.6, 112.0, 102.4, 54.3, 51.9, 47.0, 45.7, 32.5, 22.7.

LRMS (ESI) m/z: 523.3 [M+H]⁺. HRMS (ESI) m/z: calcd for C₃₁H₃₅N₆O₂, 523.2822 [M+H]⁺; found, 523.2836.

HPLC purity (method B): 96.9% (t_{R}=16.13 min).

### Example 25: N-{3-[4-(Cyclopentylamino)-6-(pyridin-4-yl)furo[2,3-d]pyrimidin-5-yl]phenyl}prop-2-enamide (25)

Compound 78n (see Scheme III) was prepared following the procedure described in Li et al., J. Med. Chem. 66, 2566-88 (2023), Supporting Information.

To a solution of 78n (71 mg, 0.19 mmol, 1 equiv) in dichloromethane (5 mL) were added acrylic acid (19 μL, 0.28 mmol, 1.4 equiv) and EDCI (52 mg, 0.27 mmol, 1.4 equiv); then, the reaction mixture was stirred at room temperature. After stirring for 16 h, the reaction mixture was quenched with NaHCO_{3 (aq)} (10 mL) and extracted with dichloromethane (10 mL×3). The combined organic layers were dried over MgSO₄, concentrated in vacuo, and purified by flash column chromatography (5% methanol in dichloromethane with 0.1% NH₄OH) to yield the title compound (70 mg, 0.16 mmol, 86%) as a yellow solid.

¹H NMR (600 MHz, DMSO-d₆) δ 10.43 (s, 1H), 8.55 (d, J=6.0 Hz, 2H), 8.41 (s, 1H), 7.97 (dd, J=2.4, 1.8 Hz, 1H), 7.82 (ddd, J=8.1, 2.4, 1.2 Hz, 1H), 7.61 (dd, J=8.1, 7.8 Hz, 1H), 7.41 (d, J=6.0 Hz, 2H), 7.30 (ddd, J=7.8, 1.8, 1.2 Hz 1H), 6.44 (dd, J=16.8, 10.2 Hz, 1H), 6.27 (dd, J=16.8, 1.8 Hz, 1H), 5.79 (dd, J=10.2, 1.8 Hz, 1H), 4.97 (d, J=6.9 Hz, 1H), 4.35 (dquin, J=6.9, 4.8 Hz, 1H), 1.88-1.77 (m, 2H), 1.53-1.33 (m, 4H), 1.27-1.17 (m, 2H).

¹³C NMR (150 MHz, DMSO-d₆) δ 164.7, 163.5, 156.9, 155.2, 150.3, 142.6, 140.3, 135.9, 131.5, 131.3, 130.6, 127.5, 124.2, 120.1, 119.5, 119.2, 119.0, 102.2, 52.1, 32.4, 22.7.

LRMS (ESI) m/z: 426.2 [M+H]⁺. HRMS (ESI) m/z: calcd for C₂₅H₂₃N₅NaO₂, 448.1749 [M+Na]⁺; found, 448.1756.

HPLC purity (method B): 96.9% (t_{R}=23.20 min).

### Example 26: N-{5-[4-(Cyclopentylamino)-6-phenylfuro[2,3-d]pyrimidin-5-yl]-2-[(2-(dimethylamino)ethyl](methyl)amino]phenyl}prop-2-enamide (26)

Compound 70n (see Scheme I) was prepared following the procedure described in Li et al., J. Med. Chem. 66, 2566-88 (2023), Supporting Information.

To a solution of 70n (28 mg, 0.06 mmol, 1 equiv) in dichloromethane (3 mL) were added DIPEA (2 μL, 0.01 mmol, 19 mol %), acrylic acid (5 μL, 0.08 mmol, 1.3 equiv), and EDCI (14 mg, 0.07 mmol, 1.2 equiv); then, the reaction mixture was stirred at room temperature. After stirring for 16 h, to the reaction mixture was added ethyl acetate (5 mL) and washed with NaHCO_{3 (aq)} (10 mL) and brine (10 mL). The combined organic layers were dried over MgSO₄, concentrated in vacuo, and purified by flash column chromatography (2-3% methanol in dichloromethane) to yield the title compound (13 mg, 0.02 mmol, 42%) as a white solid.

¹H NMR (600 MHz, DMSO-d₆) δ 10.22 (s, 1H), 8.43 (s, 1H), 8.34 (s, 1H), 7.56 (d, J=7.2 Hz, 2H), 7.47 (d, J=8.4 Hz, 1H), 7.40-7.31 (m, 3H), 7.22 (dd, J=8.4, 2.4 Hz, 1H), 6.52 (s, 1H), 6.25 (dd, J=16.8, 1.8 Hz, 1H), 5.80 (dd, J=10.2, 1.8 Hz, 1H), 4.95 (d, J=6.6 Hz, 1H), 4.35 (dquin, J=6.6, 4.2 Hz, 1H), 2.97 (s, 2H), 2.76 (s, 1H), 2.36-2.20 (m, 6H), 1.86-1.77 (m, 2H), 1.54-1.32 (m, 4H), 1.31-1.18 (m, 2H).

¹³C NMR (150 MHz, DMSO-d₆) δ 164.3, 163.4, 156.6, 154.1, 145.3, 143.6, 131.6, 129.1, 128.8, 128.7, 127.3, 126.9, 125.8, 125.3, 122.8, 115.0, 102.4, 54.9, 51.9, 41.4, 32.5, 22.8.

LRMS (ESI) m/z: 525.3 [M+H]⁺. HRMS (ESI) m/z: calcd for C₃₁H₃₇N₆O₂, 525.2978 [M+H]⁺; found, 525.2954.

HPLC purity (method B): 96.1% (t_{R}=17.10 min).

### Example 27: N-{5-[4-(Cyclopentylamino)-6-phenylfuro[2,3-d]pyrimidin-5-yl]-2-(morpholin-4-yl)phenyl}prop-2-enamide (27)

Compound 70j (see Scheme I) was prepared following the procedure described in Li et al., J. Med. Chem. 66, 2566-88 (2023), Supporting Information.

To a solution of 70j (47 mg, 0.1 mmol, 1 equiv) in dichloromethane (3 mL) were added DIPEA (4 μL, 0.02 mmol, 22 mol %), acrylic acid (9 μL, 0.14 mmol, 1.3 equiv), and EDCI (24 mg, 0.13 mmol, 1.2 equiv); then, the reaction mixture was stirred at room temperature. After stirring for 16 h, to the reaction mixture was added ethyl acetate (5 mL) and washed with NaHCO_{3 (aq)} (10 mL) and brine (10 mL). The combined organic layers were dried over MgSO₄, concentrated in vacuo, and purified by flash column chromatography (33-50% ethyl acetate in hexane) to yield the title compound (32 mg, 0.06 mmol, 61%) as a white solid.

¹H NMR (600 MHz, DMSO-d₆) δ 9.35 (s, 1H), 8.34 (s, 1H), 8.31 (d, J=1.8 Hz, 1H), 8.17 (s, 1H), 7.56-7.53 (m, 3H), 7.40-7.31 (m, 4H), 7.26 (dd, J=8.4, 1.8 Hz, 1H), 6.71 (dd, J=16.8, 10.2 Hz, 1H), 6.24 (dd, J=16.8, 1.8 Hz, 1H), 5.78 (dd, J=10.2, 1.8 Hz, 1H), 4.92 (d, J=6.9 Hz, 1H), 4.34 (dquin, J=6.9, 4.8 Hz, 1H), 3.85 (dd, J=4.8, 4.2 Hz, 4H), 2.91 (dd, J=4.8, 4.2 Hz, 4H), 1.84-1.76 (m, 2H), 1.52-1.43 (m, 2H), 1.42-1.32 (m, 2H), 1.31-1.23 (m, 2H).

¹³C NMR (150 MHz, DMSO-d₆) δ 164.3, 163.4, 156.6, 154.1, 145.3, 132.7, 131.8, 129.1, 128.9, 128.7, 127.3, 126.5, 126.1, 125.8, 123.1, 121.0, 114.9, 102.5, 79.2, 66.0, 54.9, 52.0, 51.5, 32.3, 22.7.

LRMS (ESI) m/z: 510.2 [M+H]⁺. HRMS (ESI) m/z: calcd for C₃₀H₃₂N₅O₃, 510.2505 [M+H]⁺; found, 510.2457.

HPLC purity (method B): 98.6% (t_{R}=25.93 min).

### Example 28: N-{5-[4-(Cyclopentylamino)-6-phenylfuro[2,3-d]pyrimidin-5-yl]-2-[(3R)-3-(dimethylamino)pyrrolidin-1-yl]phenyl}prop-2-enamide

Compound 28 was prepared following the procedure as described in Example 27 using (R)-N,N-dimethylpyrrolidin-3-amine.

¹H NMR (300 MHz, CD₃OD) δ 8.25 (s, 1H), 7.68 (s, 1H), 7.53-7.61 (m, 2H), 7.26-7.34 (m, 3H), 7.13-7.25 (m, 2H), 6.55 (dd, J=17.0, 10.2 Hz, 1H), 6.35 (dd, J=17.0, 1.6 Hz, 1H), 5.79 (dd, J=10.2, 1.6 Hz, 1H), 4.33-4.45 (m, 1H), 3.32-3.50 (m, 4H), 3.12-3.26 (m, 1H), 2.52 (s, 6H), 2.26-2.39 (m, 1H), 1.97-2.10 (m, 1H), 1.84-1.97 (m, 2H), 1.47-1.63 (m, 4H), 1.25-1.43 (m, 2H).

LRMS (ESI) m/z: 537.3 [M+H]⁺.

HPLC purity (method B): 93.7% (t_{R}=20.15 min).

### Example 29: (2E)-N-{3-[4-(Cyclopentylamino)-6-phenylfuro[2,3-d]pyrimidin-5-yl]phenyl}-4-(dimethylamino)but-2-enamide (29)

Compound 78f (see Scheme III) was prepared following the procedure described in Li et al., J. Med. Chem. 66, 2566-88 (2023), Supporting Information.

To a solution of 78f (50 mg, 0.13 mmol, 1 equiv) in dichloromethane (3 mL) were added 4-bromocrotonoic acid (29 mg, 0.18 mmol, 1.3 equiv) and EDCI (83 mg, 0.43 mmol, 1.5 equiv); then, the reaction mixture was stirred at room temperature. After stirring for 16 h, to the reaction mixture was added ethyl acetate (5 mL) and washed with brine (10 mL). The combined organic layers were dried over MgSO₄ and concentrated in vacuo. The residue was dissolved in THF (5.0 mL), cooled to 0° C., and added 2 M N,N-dimethylamine (202 μL, 0.41 mmol, 3.0 equiv) in THF. The reaction mixture was stirred at room temperature for 5 h, concentrated in vacuo, and purified by flash column chromatography (3-9% methanol in dichloromethane) to yield the title compound (23 mg, 0.05 mmol, 35%) as a yellow solid.

¹H NMR (600 MHz, DMSO-d₆) δ 8.35 (s, 1H), 7.86-7.81 (m, 3H), 7.59 (t, J=8.4 Hz, 1H), 7.52 (d, J=7.2 Hz, 2H), 7.40-7.29 (m, 5H), 6.38 (d, J=8.4 Hz, 1H), 5.70 (t, J=6.0 Hz, 1H), 4.84 (d, J=7.2 Hz, 1H), 4.40-4.26 (m, 1H), 3.32 (s, 6H), 1.86-1.78 (m, 2H), 1.52-1.36 (m, 4H), 1.26-1.17 (m, 2H). ¹³C NMR (150 MHz, DMSO-d₆) δ 173.6, 164.3, 156.7, 154.1, 145.4, 139.3, 131.9, 130.0, 129.0, 128.8, 128.7, 125.9, 125.8, 125.7, 122.3, 122.1, 114.9, 102.4, 52.0, 32.4, 32.3, 29.8, 28.0, 22.8.

LRMS (ESI) m/z: 482.2 [M+H]⁺. HRMS (ESI) m/z: calcd for C₂₉H₃₂N₅O₂, 482.2556 [M+H]⁺; found, 482.2557.

HPLC purity (method B): 91.6% (t_{R}=16.99 min).

### Example 30: N-{5-[4-(Cyclopentylamino)-6-phenylfuro[2,3-d]pyrimidin-5-yl]-2-[2-(dimethylamino)ethoxy]phenyl}prop-2-enamide (30)

Compound 701 (see Scheme I) was prepared following the procedure described in Li et al., J. Med. Chem. 66, 2566-88 (2023), Supporting Information.

To a solution of 701 (16 mg, 0.03 mmol, 1 equiv) in dichloromethane (5 mL) were added acrylic acid (4 μL, 0.06 mmol, 1.7 equiv) and EDCI (10 mg, 0.05 mmol, 1.5 equiv); then, the reaction mixture was stirred at room temperature. After stirring for 16 h, to the reaction mixture were added water (15 mL), dichloromethane (5 mL), and sat. NaHCO_{3(aq)} (2 mL) and extracted with dichloromethane (10 mL×3). The combined organic layers were washed with brine (10 mL), dried over MgSO₄, concentrated in vacuo, and purified by flash column chromatography (5% methanol in dichloromethane with 0.1% NH₄OH) to yield the title compound (14 mg, 0.03 mmol, 78%) as a yellow oil.

¹H NMR (600 MHz, DMSO-d₆) δ 9.76 (s, 1H), 8.34 (s, 1H), 8.31 (d, J=2.4 Hz, 1H), 7.54 (d, J=7.5 Hz, 2H), 7.37 (dd, J=8.4, 7.5 Hz, 2H), 7.35-7.30 (m, 2H), 7.21 (dd, J=8.4, 2.4 Hz, 1H), 6.60 (dd, J=16.8, 10.2 Hz, 1H), 6.23 (dd, J=16.8, 1.8 Hz, 1H), 5.77 (dd, J=10.2, 1.8 Hz, 1H), 4.99 (d, J=7.2 Hz, 1H), 4.35 (dquin, J=7.2, 5.4 Hz, 1H), 4.25 (t, J=6.0 Hz, 2H), 2.69 (t, J=6.0 Hz, 2H), 2.27 (s, 3H), 1.87-1.77 (m, 2H), 1.52-1.36 (m, 4H), 1.30-1.22 (m, 2H). ¹³C NMR (150 MHz, DMSO-d₆) δ 164.3, 163.4, 156.7, 154.1, 148.8, 145.4, 131.7, 129.5, 129.1, 128.8, 128.6, 127.2, 125.83, 125.76, 123.9, 122.1, 115.5, 114.9, 102.4, 67.9, 57.2, 52.0, 45.2, 32.4, 22.8.

LRMS (ESI) m/z: 512.3 [M+H]⁺. HRMS (ESI) m/z: calcd for C₃₀H₃₄N₅O₃, 512.2662 [M+H]⁺; found, 512.2658.

HPLC purity (method B): 99.0% (t_{R}=16.14 min).

### Example 31: N-{5-[4-(Cyclopentylamino)-6-phenylfuro[2,3-d]pyrimidin-5-yl)-2-[(1-methylpiperidin-4-yl)oxy]phenyl]prop-2-enamide (31)

Compound 70m (see Scheme I) was prepared following the procedure described in Li et al., J. Med. Chem. 66, 2566-88 (2023), Supporting Information.

To a solution of 70m (16 mg, 0.03 mmol, 1 equiv) in dichloromethane (5 mL) were added acrylic acid (3 μL, 0.04 mmol, 1.3 equiv) and EDCI (10 mg, 0.05 mmol, 1.6 equiv); then, the reaction mixture was stirred at room temperature. After stirring for 16 h, to the reaction mixture were added water (15 mL), dichloromethane (5 mL), and sat. NaHCO_{3(aq)} (2 mL) and extracted with dichloromethane (10 mL×3). The combined organic layers were washed with brine (10 mL), dried over MgSO₄, concentrated in vacuo, and purified by flash column chromatography (5% methanol in dichloromethane with 0.1% NH₄OH) to yield the title compound (9 mg, 0.02 mmol, 51%) as a yellow oil.

¹H NMR (600 MHz, DMSO-d₆) δ 9.36 (s, 1H), 8.34 (s, 1H), 8.21 (s, 1H), 7.55 (d, J=7.2 Hz, 2H), 7.37 (dd, J=7.8, 7.2 Hz, 2H), 7.35-7.30 (m, 2H), 7.20 (dd, J=8.4, 2.4 Hz, 1H), 6.72 (dd, J=17.4, 10.2 Hz, 1H), 6.22 (dd, J=17.4, 2.1 Hz, 1H), 5.76 (dd, J=10.2, 2.1 Hz, 1H), 4.95 (d, J=7.2 Hz, 1H), 4.53 (dquin, J=8.4, 4.8 Hz, 1H), 4.38-4.33 (m, 1H), 2.66 (s, 1H), 2.18 (s, 3H), 2.00-1.92 (m, 2H), 1.86-1.75 (m, 4H), 1.53-1.32 (m, 6H), 1.30-1.20 (m, 2H).

¹³C NMR (150 MHz, DMSO-d₆) δ 164.3, 163.5, 156.7, 154.1, 147.8, 145.3, 131.8, 129.2, 129.1, 128.8, 128.6, 127.1, 125.8, 125.7, 123.2, 123.0, 115.5, 114.9, 102.6, 54.9, 52.4, 51.9, 45.8, 32.4, 30.3, 22.8.

LRMS (ESI) m/z: 538.3 [M+H]⁺. HRMS (ESI) m/z: calcd for C₃₂H₃₅N₅NaO₃, 560.2638 [M+Na]⁺; found, 560.2641.

HPLC purity (method B): 96.1% (t_{R}=17.10 min).

### Example 32: N-{3-[4-(Cyclopentylamino)-6-[6-(morpholin-4-yl)pyridin-3-yl]furo[2,3-d]pyrimidin-5-yl]phenyl}prop-2-enamide (32)

Compound 78o (see Scheme III) was prepared following the procedure described in Li et al., J. Med. Chem. 66, 2566-88 (2023), Supporting Information.

To a solution of 78o (60 mg, 0.13 mmol, 1 equiv) in dichloromethane (0.7 mL) were added acrylic acid (12 μL, 0.18 mmol, 1.3 equiv) and EDCI (42 mg, 0.22 mmol, 1.7 equiv); then, the reaction mixture was stirred at room temperature. After stirring for 2 h, the reaction mixture was concentrated in vacuo and purified by flash column chromatography (5% methanol in dichloromethane) to yield the title compound (17 mg, 0.03 mmol, 25%) as a yellow solid.

¹H NMR (600 MHz, DMSO-d₆) δ 10.38 (s, 1H), 8.32 (s, 1H), 8.24 (dd, J=2.1, 0.9 Hz, 1H), 7.92 (dd, J=2.1, 1.8 Hz, 1H), 7.70 (ddd, J=8.1, 2.1, 1.2 Hz, 1H), 7.60 (dd, J=9.3, 2.4 Hz, 1H), 7.55 (dd, J=8.1, 7.8 Hz, 1H), 7.23 (ddd, J=7.8, 1.8, 1.2 Hz, 1H), 6.85 (dd, J=9.3, 0.9 Hz, 1H), 6.43 (dd, J=16.8, 10.2 Hz, 1H), 6.27 (dd, J=16.8, 1.8 Hz, 1H), 5.78 (dd, J=10.2, 1.8 Hz, 1H), 4.90 (d, J=6.9 Hz, 1H), 4.34 (dquin, J=6.9, 4.8 Hz, 1H), 3.65 (dd, J=6.0, 5.1 Hz, 4H), 3.48 (dd, J=6.0, 5.1 Hz, 4H), 1.87-1.77 (m, 2H), 1.54-1.36 (m, 4H), 1.26-1.18 (m, 2H).

¹³C NMR (150 MHz, DMSO-d₆) δ 164.3, 163.4, 158.2, 156.3, 153.6, 145.5, 144.6, 140.1, 134.9, 132.1, 131.5, 130.3, 127.4, 124.5, 119.9, 119.5, 114.5, 112.8, 106.6, 102.1, 65.8, 52.0, 44.6, 32.5, 22.7.

LRMS (ESI) m/z: 511.3 [M+H]⁺. HRMS (ESI) m/z: calcd for C₂₉H₃₀N₆NaO₃, 533.2277 [M+Na]⁺; found, 533.2278.

HPLC purity (method B): 99.8% (t_{R}=24.56 min).

### Example 33: N-{3-[4-(Cyclopentylamino)-6-[6-(4-methylpiperazin-1-yl)pyridin-3-yl]furo[2,3-d]pyrimidin-5-yl]phenyl}prop-2-enamide (33)

Compound 78p (see Scheme III) was prepared following the procedure described in Li et al., J. Med. Chem. 66, 2566-88 (2023), Supporting Information.

To a solution of 78p (75 mg, 0.16 mmol, 1 equiv) in dichloromethane (0.8 mL) were added acrylic acid (13 μL, 0.19 mmol, 1.2 equiv) and EDCI (46 mg, 0.24 mmol, 1.5 equiv); then, the reaction mixture was stirred at room temperature. After stirring for 2 h, the reaction mixture was concentrated in vacuo, dissolved in ethyl acetate, and washed with NaHCO_{3(aq)} (10 mL) and brine (10 mL). The combined organic layers were dried over MgSO₄, concentrated in vacuo, and purified by flash column chromatography (5% methanol in dichloromethane) to yield the title compound (12 mg, 0.02 mmol, 14%) as a yellow solid.

¹H NMR (600 MHz, DMSO-d₆) δ 10.38 (s, 1H), 8.31 (s, 1H), 8.22 (d, J=2.4 Hz, 1H), 7.91 (dd, J=1.8, 1.8 Hz, 1H), 7.77 (dd, J=8.4, 1.8 Hz, 1H), 7.57-7.53 (m, 2H), 7.24-7.21 (m, 1H), 6.84 (d, J=9.0 Hz, 1H), 6.43 (dd, J=17.1, 10.2 Hz, 1H), 6.27 (dd, J=17.1, 1.8 Hz, 1H), 5.78 (dd, J=10.2, 1.8 Hz, 1H), 4.89 (d, J=7.2 Hz, 1H), 4.34 (dquin, J=7.2, 4.8 Hz, 1H), 3.51 (dd, J=5.1, 4.8 Hz, 4H), 2.35 (dd, J=5.1, 4.8 Hz, 4H), 2.19 (s, 3H), 1.87-1.77 (m, 2H), 1.53-1.35 (m, 4H), 1.26-1.17 (m, 2H).

¹³C NMR (150 MHz, DMSO-d₆) δ 164.3, 163.5, 158.0, 156.2, 153.5, 145.5, 144.7, 140.1, 134.8, 132.2, 131.6, 130.3, 127.4, 124.5, 119.9, 119.5, 114.0, 112.6, 106.6, 102.2, 54.2, 52.0, 45.7, 44.1, 32.5, 22.7.

LRMS (ESI) m/z: 524.3 [M+H]⁺. HRMS (ESI) m/z: calcd for C₃₀H₃₃N₇NaO₂, 546.2593 [M+Na]⁺; found, 546.2593.

HPLC purity (method B): 95.7% (t_{R}=15.56 min).

### Example 34: N-(5-[4-(Cyclopropylamino)-6-phenylfuro[2,3-d]pyrimidin-5-yl]-2-{[2-(dimethylamino)ethyl](methyl)amino}phenyl)prop-2-enamide

Compound 34 was prepared following the procedure as described in Example 26 using cyclopentanamine.

¹H NMR (400 MHz, CDCl₃) δ10.33 (s, 1H), 8.70 (s, 1H), 8.50 (s, 1H), 7.59-7.56 (m, 2H), 7.30-7.21 (m, 4H), 7.08 (dd, J=8.0, 2.0 Hz, 1H), 6.44 (dd, J=16.8, 1.6 Hz, 1H), 6.42-6.34 (m, 1H), 5.74 (dd, J=10.0, 1.6 Hz, 1H), 5.18 (s, 1H), 2.93-2.90 (m, 2H), 2.88-2.82 (m, 1H), 2.80 (s, 3H), 2.40-2.44 (m, 2H), 2,34 (s, 6H), 0.79-0.74 (m, 2H), 0.42-0.39 (m, 2H) LRMS (ESI) m/z: 497.4 [M+H]⁺.

HPLC purity (method B): 96.4% (t_{R}=14.46 min).

### Example 35: N-(2-{[2-(Dimethylamino)ethyl](methyl)amino}-5-{4-[(2-hydroxyethyl)amino]-6-phenylfuro[2,3-d]pyrimidin-5-yl}phenyl)prop-2-enamide (35)

Compound 70s (see Scheme I) was prepared following the procedure described in Li et al., J. Med. Chem. 66, 2566-88 (2023), Supporting Information.

To a solution of 70s (56 mg, 0.13 mmol, 1 equiv) in dichloromethane (5 mL) were added acrylic acid (9 μL, 0.13 mmol, 1.1 equiv) and EDCI (36 mg, 0.19 mmol, 1.5 equiv); then, the reaction mixture was stirred at room temperature. After stirring for 16 h, the reaction mixture was concentrated in vacuo and purified by flash column chromatography (2-5% methanol in dichloromethane with 0.1% NH₄OH) to yield the title compound (36 mg, 0.07 mmol, 57%) as a yellow solid.

¹H NMR (600 MHz, DMSO-d₆) δ 10.32 (s, 1H), 8.36 (d, J=2.1 Hz, 1H), 8.33 (s, 1H), 7.52 (d, J=7.2 Hz, 2H), 7.41 (d, J=8.1 Hz, 1H), 7.38-7.30 (m, 3H), 7.19 (dd, J=8.1, 2.1 Hz, 1H), 6.44 (dd, J=17.1, 10.2 Hz, 1H), 6.24 (dd, J=17.1, 2.1 Hz, 1H), 5.79 (dd, J=10.2, 2.1 Hz, 1H), 5.50 (t, J=5.4 Hz, 1H), 3.50-3.38 (m, 4H), 2.91 (t, J=6.0 Hz, 2H), 2.76 (s, 3H), 2.40 (t, J=6.0 Hz, 2H), 2.23 (s, 6H). ¹³C NMR (150 MHz, DMSO-d₆) δ 164.5, 163.4, 157.2, 154.0, 145.3, 143.7, 134.4, 131.7, 129.2, 128.8, 128.6, 127.2, 126.5, 126.0, 125.1, 122.7, 121.6, 115.3, 102.3, 59.1, 56.7, 55.7, 45.5, 42.8, 41.1.

LRMS (ESI) m/z: 501.4 [M+H]⁺. HRMS (ESI) m/z: calcd for C₂₈H₃₃N₆O₃, 501.2614 [M+H]⁺; found, 501.2593.

HPLC purity (method B): 93.4% (t_{R}=13.60 min).

### Example 36: N-[5-(4-{[2-(4-Hydroxyphenyl)ethyl]amino}-6-phenylfuro[2,3-d]pyrimidin-5-yl)-2-(4-methylpiperazin-1-yl)phenyl]prop-2-enamide

Compound 36 was prepared following the procedure as described in Example 38 using 4-(2-aminoethyl)phenol.

¹H NMR (400 MHz, DMSO-d₆) δ 9.23 (s, 1H), 8.35 (s, 1H), 8.13 (s, 1H), 7.56 (d, J=7.2 Hz, 2H), 7.39-7.31 (m, 4H), 7.23 (dd, J=8.4, 2.4 Hz, 1H), 6.66 (dd, J=16.8, 10.2 Hz, 1H), 6.22 (dd, J=16.8, 1.8 Hz, 1H), 5.76 (dd, J=10.2, 1.8 Hz, 1H), 5.52 (t, J=5.4 Hz, 1H), 4.90 (d, J=6.9 Hz, 1H), 4.64 (t, J=4.8 Hz, 1H), 3.49-3.41 (m, 4H), 2.91 (s, 4H), 2.57 (s, 4H), 2.28 (s, 3H).

LRMS (ESI) m/z: 575.3 [M+H]⁺.

HPLC purity (method B): 94.0% (t_{R}=16.05 min).

### Example 37: N-[2-{[2-(Dimethylamino)ethyl](methyl)amino}-5-(4-{[2-(4-hydroxyphenyl)ethyl]amino}-6-phenylfuro[2,3-d]pyrimidin-5-yl)phenyl]prop-2-enamide

Compound 37 was prepared following the procedure as described in Example 26 using 4-(2-aminoethyl)phenol.

¹H NMR (300 MHz, CDCl₃) δ 8.96 (br, 1H), 8.40 (s, 1H), 8.33 (s, 1H), 7.45-7.42 (m, 2H), 7.24-7.19 (m, 3H), 7.05 (d, J=8.1 Hz, 1H), 6.97 (dd, J=8.1, 1.8 Hz, 1H), 6.71 (d, J=10.5 Hz, 1H), 6.51-6.45 (m, 4H), 5.77 (dd, J=12.0, 6.0 Hz, 1H), 4.94 (t, J=5.4 Hz, 1H), 3.82-3.86 (m, 2H), 3.47-3.51 (m, 4H), 2.75-2.67 (m, 7H), 2.47 (s, 6H)

LRMS (ESI) m/z: 577.4 [M+H]⁺.

HPLC purity (method B): 97.3% (t_{R}=16.50 min).

### Example 38: N-{5-[4-(Cyclopentylamino)-6-phenylfuro[2,3-d]pyrimidin-5-yl]-2-(4-methylpiperazin-1-yl)phenyl}prop-2-enamide (38)

Compound 70 k (see Scheme I) was prepared following the procedure described in Li et al., J. Med. Chem. 66, 2566-88 (2023), Supporting Information.

To a solution of 70 k (51 mg, 0.1 mmol, 1 equiv) in dichloromethane (5 mL) were added acrylic acid (8 μL, 0.11 mmol, 1.1 equiv) and EDCI (31 mg, 0.16 mmol, 1.6 equiv); then, the reaction mixture was stirred at room temperature. After stirring for 16 h, the reaction mixture was concentrated in vacuo and purified by thin-plate chromatography (5% methanol in dichloromethane with 0.1% NH₄OH) to yield the title compound (18 mg, 0.03 mmol, 34%) as a yellow solid.

¹H NMR (600 MHz, DMSO-d₆) δ 9.24 (s, 1H), 8.34 (s, 1H), 8.12 (s, 1H), 7.55 (d, J=7.2 Hz, 2H), 7.40-7.30 (m, 4H), 7.24 (dd, J=8.4, 2.4 Hz, 1H), 6.67 (dd, J=16.8, 10.2 Hz, 1H), 6.23 (dd, J=16.8, 1.8 Hz, 1H), 5.77 (dd, J=10.2, 1.8 Hz, 1H), 4.91 (d, J=6.9 Hz, 1H), 4.34 (dquin, J=6.9, 2.4 Hz, 1H), 2.92 (s, 4H), 2.58 (s, 4H), 2.27 (s, 3H), 1.84-1.75 (m, 2H), 1.52-1.22 (m, 6H).

¹³C NMR (150 MHz, DMSO-d₆) δ 164.3, 163.3, 156.6, 154.1, 145.3, 144.2, 132.5, 131.8, 129.1, 128.8, 128.7, 127.2, 126.2, 126.0, 125.8, 123.1, 120.9, 114.9, 102.6, 54.5, 52.0, 50.9, 45.7, 32.4, 22.7.

LRMS (ESI) m/z: 523.4 [M+H]⁺. HRMS (ESI) m/z: calcd for C₃₁H₃₅N₆O₂, 523.2822 [M+H]⁺; found, 523.2829.

HPLC purity (method B): 98.6% (t_{R}=16.83 min).

### Example 39: N-(5-[4-(Cyclopentylamino)-6-phenylfuro[2,3-d]pyrimidin-5-yl]-2-{methyl[2-(4-methylpiperazin-1-yl)ethyl]amino}phenyl)prop-2-enamide

Compound 39 was prepared following the procedure as described in Example 26 using N-methyl-2-(4-methylpiperazin-1-yl)ethan-1-amine.

¹H NMR (400 MHz, CD₃OD) δ 8.42 (s, 1H), 8.28 (s, 1H), 7.60-7.57 (m, 2H), 7.47 (d, J=8.0 Hz, 1H), 7.30-7.32 (m, 3H), 7.22 (dd, J=8.0, 2.0 Hz, 1H), 6.64 (dd, J=16.8, 10.4 Hz, 2H), 6.37 (d, J=16.8, 1.6 Hz, 1H), 5.83 (d, J=10.4, 1.2 Hz, 1H), 4.41-4.38 (m, 1H), 3.68-3.66 (m, 2H), 3.17 (t, J=6.4 Hz, 2H), 2.77 (s, 3H), 2.58-2.53 (m, 8H), 2.47 (s, 3H), 1.95-1.87 (m, 2H), 1.58-1.52 (m, 2H), 1.48-1.45 (m, 2H), 1.28-1.24 (m, 2H)

LRMS (ESI) m/z: 580.3 [M+H]⁺.

HPLC purity (method B): 97.1% (t_{R}=17.25 min).

### Example 40: N-[5-(4-{[2-(4-Hydroxyphenyl)ethyl]amino}-6-phenylfuro[2,3-d]pyrimidin-5-yl)-2-{methyl [2-(4-methylpiperazin-1-yl)ethyl]amino}phenyl]prop-2-enamide

Compound 40 was prepared following the procedure as described in Example 37 using N-methyl-2-(4-methylpiperazin-1-yl)ethan-1-amine.

¹H NMR (400 MHz, CD₃OD) δ 8.30 (s, 1H), 8.19 (s, 1H), 7.54-7.51 (m, 2H), 7.31-7.28 (m, 4H), 7.07 (dd, J=8.0, 2.0 Hz, 1H), 6.76 (d, J=8.0 Hz, 2H), 6.67-6.57 (m, 3H), 6.37 (dd, J=16.8, 1.2 Hz, 1H), 5.82 (dd, J=10.4, 1.2 Hz, 1H), 3.64-3.56 (m, 4H), 3.14 (t, J=6.4 Hz, 2H), 2.77 (s, 3H), 2.67 (t, J=6.4 Hz, 2H), 2.56 (t, J=6.4 Hz, 2H), 2.53-2.50 (m, 2H), 2.27 (s, 3H)

LRMS (ESI) m/z: 632.3 [M+H]⁺.

HPLC purity (method B): 98.0% (t_{R}=15.83 min).

### Example 41: N-(5-[4-({2-[4-(Aminosulfonyl)phenyl]ethyl}amino)-6-phenylfuro-[2,3-d]pyrimidin-5-yl]-2-{[2-(dimethylamino)ethyl](methyl)amino}phenyl)prop-2-enamide

Compound 41 was prepared following the procedure as described in Example 26 using 4-(2-aminoethyl)benzene-1-sulfonamide.

¹H NMR (300 MHz, CD₃OD) δ 8.31 (s, 1H), 8.17 (s, 1H), 7.75 (d, J=6.9 Hz, 2H), 7.54-7.50 (m, 2H), 7.32-7.23 (m, 6H), 7.01 (dd, J=8.1, 2.1 Hz, 1H), 6.57 (dd, J=16.8, 10.2 Hz, 1H), 6.38 (dd, J=16.8, 1.8 Hz, 1H), 5.82 (dd, J=10.2, 1.8 Hz, 1H), 3.78 (t, J=6.6 Hz, 2H), 3.08 (t, J=6.0 Hz, 2H), 2.94 (t, J=6.6 Hz, 2H), 2.72 (s, 3H), 2.57 (t, J=6.0 Hz, 2H), 2.32 (s, 3H).

LRMS (ESI) m/z: 640.8 [M+H]⁺.

HPLC purity (method B): 94.2% (t_{R}=15.35 min).

### Example 42: N-(5-[4-(Cyclopentylamino)-6-(4-fluorophenyl)furo[2,3-d]pyrimi-din-5-yl]-2-{[2-(dimethylamino)ethyl](methyl)amino}phenyl)prop-2-enamide

Compound 42 was prepared following the procedure as described in Example 26 using 1-ethynyl-4-fluorobenzene.

¹H NMR (300 MHz, CDCl₃) δ 10.34 (br, 1H), 8.79 (s, 1H), 8.39 (s, 1H), 7.59 (dd, J=7.5, 5.4 Hz, 2H), 7.33 (d, J=8.1 Hz, 1H), 7.10 (d, J=8.1 Hz, 1H), 6.99-6.94 (m, 2H), 6.47-6.29 (m, 2H), 5.74 (d, J=10.2 Hz, 1H), 4.87-4.85 (m, 1H), 4.43-4.37 (m, 1H), 2.91 (br, 2H), 2.80 (s, 3H), 2.42 (br, 2H), 2.33 (s, 6H), 1.93-1.87 (m, 2H), 1.56-1.43 (m, 4H), 1.25 (m, 2H).

LRMS (ESI) m/z: 543.6 [M+H]⁺.

HPLC purity (method B): 99.9% (t_{R}=17.61 min).

### Example 43: N-(2-{[2-(Dimethylamino)ethyl](methyl)amino}-5-[6-phenyl-4-(phenylamino)furo[2,3-d]pyrimidin-5-yl]phenyl)prop-2-enamide

Compound 43 was prepared following the procedure as described in Example 26 using aniline.

¹H NMR (300 MHz, CDCl₃) δ 10.39 (br, 1H), 8.91 (s, 1H), 8.53 (s, 1H), 7.65-7.61 (m, 2H), 7.44-7.37 (m, 3H), 7.30-7.18 (m, 5H), 7.02 (t, J=7.5 Hz, 1H), 6.86 (s, 1H), 6.47-6.29 (m, 2H), 5.76-5.72 (m, 1H), 3.18 (br, 2H), 2.95 (s, 3H), 2.61 (br, 2H), 2.45 (s, 6H).

LRMS (ESI) m/z: 533.6 [M+H]⁺.

UPLC purity: 99.4% (t_{R}=2.24 min).

### Example 44: N-{3-[4-(Methylamino)-6-[4-(4-methylpiperazin-1-yl)phenyl]furo[2,3-d]pyrimidin-5-yl]phenyl}prop-2-enamide (44)

Compound 78m (see Scheme III) was prepared following the procedure described in Li et al., J. Med. Chem. 66, 2566-88 (2023), Supporting Information.

To a solution of 78m (58 mg, 0.14 mmol, 1 equiv) in dichloromethane (1 mL) were added acrylic acid (12 μL, 0.18 mmol, 1.3 equiv) and EDCI (40 mg, 0.21 mmol, 1.5 equiv); then, the reaction mixture was stirred at room temperature. After stirring for 1 h, the reaction mixture was quenched with NaHCO_{3 (aq)} (10 mL) and extracted with ethyl acetate (10 mL×3). The combined organic layers were dried over MgSO₄, concentrated in vacuo, and purified by flash column chromatography (5% methanol in dichloromethane) to yield the title compound (27 mg, 0.06 mmol, 41%) as colorless oil.

¹H NMR (600 MHz, DMSO-d₆) δ 10.34 (s, 1H), 8.31 (s, 1H), 7.84 (ddd, J=8.1, 1.8, 1.2 Hz, 1H), 7.72 (dd, J=1.8, 1.8 Hz, 1H), 7.50 (dd, J=8.1, 7.8 Hz, 1H), 7.27 (d, J=9.0 Hz, 2H), 7.15 (d, J=7.8, 1.8, 1.2 Hz 1H), 6.88 (d, J=9.0 Hz, 2H), 6.44 (dd, J=16.8, 10.2 Hz, 1H), 6.27 (dd, J=16.8, 1.8 Hz, 1H), 5.77 (dd, J=10.2, 1.8 Hz, 1H), 5.42 (q, J=4.8 Hz, 1H), 3.17 (dd, J=5.4, 4.8 Hz, 4H), 2.87 (d, J=4.8 Hz, 3H), 2.39 (dd, J=5.4, 4.8 Hz, 4H), 2.19 (s, 3H).

¹³C NMR (150 MHz, DMSO-d₆) δ 164.1, 163.4, 157.3, 153.2, 150.7, 146.4, 139.9, 132.3, 131.7, 130.2, 127.2, 127.0, 124.8, 120.4, 119.6, 118.5, 114.4, 112.4, 102.3, 54.3, 46.9, 45.7, 27.9.

LRMS (ESI) m/z: 469.2 [M+H]⁺. HRMS (ESI) m/z: calcd for C₂₇H₂₈N₆NaO₂, 491.2171 [M+Na]⁺; found, 491.2173.

HPLC purity (method B): 99.4% (t_{R}=13.31 min).

### Example 45: N-(2-{[2-(Dimethylamino)ethyl](methyl)amino}-5-[4-(ethylamino)-6-phenylfuro[2,3-d]pyrimidin-5-yl]phenyl)prop-2-enamide (45)

Compound 70p (see Scheme I) was prepared following the procedure described in Li et al., J. Med. Chem. 66, 2566-88 (2023), Supporting Information.

To a solution of 70p (18 mg, 0.04 mmol, 1 equiv) in dichloromethane (5 mL) were added acrylic acid (4 μL, 0.06 mmol, 1.4 equiv) and EDCI (12 mg, 0.06 mmol, 1.5 equiv); then, the reaction mixture was stirred at room temperature. After stirring for 4 h, to the reaction mixture were added water (3 mL), dichloromethane (10 mL), and sat. NaHCO_{3(aq)} (1 mL) and extracted with dichloromethane (10 mL×3). The combined organic layers were washed with brine (10 mL), dried over MgSO₄, concentrated in vacuo, and purified by flash column chromatography (5% methanol in dichloromethane with 0.1% NH₄OH) to yield the title compound (10 mg, 0.02 mmol, 49%) as a yellow solid.

¹H NMR (400 MHz, DMSO-d₆) δ 10.33 (s, 1H), 8.69 (s, 1H), 8.40 (s, 1H), 7.58 (d, J=8.0 Hz, 2H), 7.32-7.23 (m, 5H), 7.11 (dd, J=8.4, 2.4 Hz, 1H), 6.44 (dd, J=17.2, 2.0 Hz, 1H), 6.35 (s, 1H), 5.74 (dd, J=10.0, 2.4 Hz, 1H), 5.07 (s, 1H), 3.49 (dq, J=7.2, 5.2 Hz, 2H), 2.94 (s, 2H), 2.80 (s, 3H), 2.50-2.24 (m, 8H), 1.09 (t, J=7.2 Hz, 3H).

¹³C NMR (150 MHz, DMSO-d₆) δ 164.5, 163.5, 157.0, 154.0, 145.4, 144.0, 133.9, 131.6, 129.2, 128.8, 128.7, 127.3, 126.1, 125.6, 122.5, 115.2, 102.1, 56.6, 55.2, 45.4, 41.0, 35.4, 14.2.

LRMS (ESI) m/z: 485.3 [M+H]⁺. HRMS (ESI) m/z: calcd for C₂₈H₃₃N₆O₂, 485.2665 [M+H]⁺; found, 485.2665.

HPLC purity (method B): 98.0% (t_{R}=15.32 min).

### Example 46: N-(2-{[2-(Dimethylamino)ethyl](methyl)amino}-5-[4-(methylamino)-6-phenylfuro[2,3-d]pyrimidin-5-yl]phenyl)prop-2-enamide (46)

Compound 70o (see Scheme I) was prepared following the procedure described in Li et al., J. Med. Chem. 66, 2566-88 (2023), Supporting Information.

To a solution of 70o (26 mg, 0.06 mmol, 1 equiv) in dichloromethane (5 mL) were added acrylic acid (6 μL, 0.09 mmol, 1.4 equiv) and EDCI (17 mg, 0.09 mmol, 1.4 equiv); then, the reaction mixture was stirred at room temperature. After stirring for 4 h, to the reaction mixture were added water (3 mL), dichloromethane (10 mL), and sat. NaHCO_{3(aq)} (1 mL) and extracted with dichloromethane (10 mL×3). The combined organic layers were washed with brine (10 mL), dried over MgSO₄, concentrated in vacuo, and purified by flash column chromatography (5% methanol in dichloromethane with 0.1% NH₄OH) to yield the title compound (12 mg, 0.03 mmol, 41%) as a yellow solid.

¹H NMR (400 MHz, DMSO-d₆) δ 10.41 (s, 1H), 8.54 (s, 1H), 8.43 (s, 1H), 7.58-7.53 (m, 3H), 7.30-7.20 (m, 4H), 7.10 (dd, J=9.8, 2.4 Hz, 1H), 6.45 (d, J=16.8 Hz, 1H), 5.75 (dd, J=9.8 Hz, 1H), 5.41 (s, 1H), 3.05 (d, J=4.8 Hz, 3H), 2.95 (s, 2H), 2.80 (s, 3H), 2.50-2.26 (m, 8H).

¹³C NMR (150 MHz, DMSO-d₆) δ 164.5, 163.6, 157.7, 153.9, 145.4, 133.7, 131.6, 129.2, 128.7, 128.6, 128.4, 128.2, 127.3, 126.2, 125.6, 122.5, 122.4, 115.4, 102.2, 56.7, 55.4, 45.5, 28.1, 24.9.

LRMS (ESI) m/z: 471.2 [M+H]⁺. HRMS (ESI) m/z: calcd for C₂₇H₃₁N₆O₂, 471.2509 [M+H]⁺; found, 471.2539.

HPLC purity (method B): 92.2% (t_{R}=14.44 min).

### Example 47: N-(2-{[2-(Dimethylamino)ethyl](methyl)amino}-5-[6-phenyl-4-(propan-2-ylamino)furo[2,3-d]pyrimidin-5-yl]phenyl)prop-2-enamide (47)

Compound 70q (see Scheme I) was prepared following the procedure described in Li et al., J. Med. Chem. 66, 2566-88 (2023), Supporting Information.

To a solution of 70q (19 mg, 0.04 mmol, 1 equiv) in dichloromethane (5 mL) were added acrylic acid (4 μL, 0.06 mmol, 1.4 equiv) and EDCI (12 mg, 0.06 mmol, 1.5 equiv); then, the reaction mixture was stirred at room temperature. After stirring for 4 h, to the reaction mixture were added water (3 mL), dichloromethane (10 mL), and sat. NaHCO_{3(aq)} (1 mL) and extracted with dichloromethane (10 mL×3). The combined organic layers were washed with brine (10 mL), dried over MgSO₄, concentrated in vacuo, and purified by flash column chromatography (5% methanol in dichloromethane with 0.1% NH₄OH) to yield the title compound (10 mg, 0.02 mmol, 47%) as a yellow solid.

¹H NMR (600 MHz, DMSO-d₆) δ 10.27 (s, 1H), 8.41 (d, J=2.1 Hz, 1H), 8.34 (s, 1H), 7.55 (d, J=6.6 Hz, 2H), 7.45 (d, J=8.4 Hz, 1H), 7.41-7.31 (m, 3H), 7.19 (dd, J=8.4, 2.1 Hz, 1H), 6.47 (dd, J=17.4, 10.2 Hz, 1H), 6.25 (dd, J=17.4, 2.1 Hz, 1H), 5.80 (dd, J=10.2, 2.1 Hz, 1H), 4.93 (d, J=7.2 Hz, 1H), 4.17 (dset, J=7.2, 6.6, 6.3 Hz, 1H), 2.93 (t, J=6.0 Hz, 2H), 2.78 (s, 3H), 2.38 (t, J=6.0 Hz, 2H), 2.21 (s, 6H), 1.03 (d, J=6.3 Hz, 6H).

¹³C NMR (150 MHz, DMSO-d₆) δ 164.4, 163.4, 156.5, 154.1, 145.3, 143.7, 134.4, 131.6, 129.1, 128.8, 128.7, 127.3, 126.6, 126.0, 125.4, 122.7, 121.5, 115.1, 102.2, 56.7, 55.5, 45.4, 42.3, 41.3, 22.0.

LRMS (ESI) m/z: 499.3 [M+H]⁺. HRMS (ESI) m/z: calcd for C₂₉H₃₅N₆O₂, 499.2822 [M+H]⁺; found, 499.2824.

HPLC purity (method B): 99.0% (t_{R}=16.31 min).

### Example 48: N-(2-{[2-(Dimethylamino)ethyl](methyl)amino}-5-{4-[((1S,2R)-2-hydroxycyclopentyl)amino]-6-phenylfuro[2,3-d]pyrimidin-5-yl}phenyl)prop-2-enamide (48)

Compound 70t (see Scheme I) was prepared following the procedure described in Li et al., J. Med. Chem. 66, 2566-88 (2023), Supporting Information.

To a solution of 70t (88 mg, 0.18 mmol, 1 equiv.) in dichloromethane (0.9 mL) was added acrylic acid (15 μL, 0.22 mmol, 1.2 equiv.) and EDCI (52 mg, 0.27 mmol, 1.5 equiv.), then the reaction mixture was stirred at room temperature. After stirred for 16 hours, the reaction mixture concentrated in vacuo and purified by flash column chromatography (3% methanol in dichloromethane) to yield the title compound (55 mg, 0.1 mmol, 56%) as yellow solid.

¹H NMR (600 MHz, DMSO-d₆) δ 10.32 (s, 1H), 8.45 (s, 1H), 8.31 (s, 1H), 7.55 (d, J=8.4 Hz, 2H), 7.44 (d, J=8.4 Hz, 1H), 7.38-7.29 (m, 3H), 7.20 (dd, J=8.4, 2.4 Hz, 1H), 6.41 (dd, J=16.8, 10.2 Hz, 1H), 6.22 (dd, J=16.8, 2.1 Hz, 1H), 5.77 (dd, J=10.2, 2.1 Hz, 1H), 5.63 (d, J=7.2 Hz, 1H), 4.63 (s, 1H), 4.15 (t, J=7.2 Hz, 1H), 3.94-3.88 (m, 1H), 2.95-2.84 (m, 2H), 2.76 (s, 3H), 2.39 (t, J=5.4 Hz, 2H), 2.33 (s, 6H), 2.00-1.85 (m, 1H), 1.76-1.68 (m 1H), 1.64-1.54 (m, 1H), 1.46-1.29 (m, 3H).

¹³C NMR (150 MHz, DMSO-d₆) δ 164.4, 163.3, 156.8, 154.1, 145.1, 143.3, 134.9, 131.8, 129.2, 128.8, 128.6, 127.1, 126.8, 125.8, 124.8, 122.9, 115.5, 102.5, 70.3, 56.7, 55.9, 54.4, 45.4, 41.3, 32.3, 29.4, 20.0.

LRMS (ESI) m/z: 541.3 [M+H]⁺. HRMS (ESI) m/z: calcd for C₃₁H₃₇N₆O₃, 541.2927 [M+H]+, found 541.2916.

HPLC purity (method B): 98.2% (t_{R}=15.07 min).

### Example 49: N-(2-{[2-(Dimethylamino)ethyl](methyl)amino}-5-{4-[(3-hydroxy-cyclobutyl)amino]-6-phenylfuro[2,3-d]pyrimidin-5-yl}phenyl)prop-2-enamide (49)

Compound 70v (see Scheme I) was prepared following the procedure described in Li et al., J. Med. Chem. 66, 2566-88 (2023), Supporting Information.

To a solution of 70v (75 mg, 0.16 mmol, 1 equiv) in dichloromethane (0.8 mL) were added acrylic acid (13 μL, 0.19 mmol, 1.2 equiv) and EDCI (46 mg, 0.24 mmol, 1.5 equiv); then, the reaction mixture was stirred at room temperature. After stirring for 16 h, the reaction mixture was concentrated in vacuo and purified by flash column chromatography (3% methanol in dichloromethane) to yield the title compound (35 mg, 0.07 mmol, 42%) as a white solid.

¹H NMR (600 MHz, DMSO-d₆) δ 10.34 (s, 1H), 8.40 (d, J=2.4 Hz, 1H), 8.33 (s, 1H), 7.54 (d, J=7.2 Hz, 2H), 7.46 (d, J=8.1 Hz, 1H), 7.39-7.31 (m, 3H), 7.21 (dd, J=8.1, 2.4 Hz, 1H), 6.46 (dd, J=17.4, 10.2 Hz, 1H), 6.27 (dd, J=17.4, 1.8 Hz, 1H), 5.80 (dd, J=10.2, 1.8 Hz, 1H), 5.21 (d, J=7.2 Hz, 1H), 5.00 (s, 1H), 4.03-3.95 (m, 1H), 3.81 (ddd, J=7.2, 7.2, 7.2 Hz, 1H), 2.94 (t, J=6.0 Hz, 2H), 2.79 (s, 3H), 2.60-2.53 (m, 2H), 2.41 (t, J=6.0 Hz, 2H), 2.22 (s, 6H), 1.51-1.44 (m 2H).

¹³C NMR (150 MHz, DMSO-d₆) δ 164.5, 163.5, 156.3, 154.0, 145.5, 143.9, 134.3, 131.6, 129.1, 128.8, 128.7, 127.4, 126.5, 126.0, 125.4, 122.6, 121.7, 115.1, 102.3, 59.1, 56.8, 55.6, 45.5, 41.4, 41.2, 37.3.

LRMS (ESI) m/z: 527.3 [M+H]⁺. HRMS (ESI) m/z: calcd for C₃₀H₃₄N₆NaO₃, 549.2590 [M+Na]⁺; found, 549.2589.

HPLC purity (method B): 98.1% (t_{R}=14.06 min).

### Example 50: N-{3-[4-(Cyclohexylamino)-6-[4-(4-methylpiperazin-1-yl)phenyl]-furo[2,3-d]pyrimidin-5-yl]phenyl}prop-2-enamide (50)

Compound 781 (see Scheme III) was prepared following the procedure described in Li et al., J. Med. Chem. 66, 2566-88 (2023), Supporting Information.

To a solution of 781 (130 mg, 0.27 mmol, 1 equiv) in dichloromethane (5 mL) at 0° C. were added acrylic acid (28 μL, 0.41 mmol, 1.5 equiv) and EDCI (77 mg, 0.4 mmol, 1.5 equiv); then, the reaction mixture was stirred at room temperature. After stirring for 16 h, the reaction mixture was quenched with NaHCO_{3(aq)} (10 mL) and extracted with dichloromethane (10 mL×3). The combined organic layers were dried over MgSO₄, concentrated in vacuo, and purified by flash column chromatography (5% methanol in dichloromethane with 0.1% NH₄OH) to yield the title compound (67 mg, 0.12 mmol, 46%) as a yellow solid.

¹H NMR (600 MHz, DMSO-d₆) δ 10.37 (s, 1H), 8.28 (s, 1H), 7.91 (dd, J=2.4, 1.8 Hz, 1H), 7.80 (dd, J=8.1, 1.8 Hz, 1H), 7.55 (dd, J=8.1, 7.8 Hz, 1H), 7.34 (d, J=9.0 Hz, 2H), 7.22 (d, J=7.8 Hz 1H), 6.90 (d, J=9.0 Hz, 2H), 6.44 (dd, J=16.8, 10.2 Hz, 1H), 6.27 (dd, J=16.8, 2.1 Hz, 1H), 5.77 (dd, J=10.2, 2.1 Hz, 1H), 4.86 (d, J=7.8 Hz, 1H), 4.02-3.92 (m, 1H), 3.17 (dd, J=5.4, 4.8 Hz, 4H), 2.40 (dd, J=5.4, 4.8 Hz, 4H), 2.19 (s, 3H), 1.77-1.69 (m, 2H), 1.45-1.22 (m, 5H), 1.17-1.02 (m, 3H).

¹³C NMR (150 MHz, DMSO-d₆) δ 164.1, 163.4, 156.0, 153.4, 150.8, 146.4, 140.1, 132.7, 131.6, 130.3, 127.4, 126.9, 124.6, 119.9, 119.4, 118.5, 114.5, 112.0, 102.3, 54.3, 47.8, 47.0, 45.7, 31.4, 25.0, 23.1.

LRMS (ESI) m/z: 537.2 [M+H]⁺. HRMS (ESI) m/z: calcd for C₃₂H₃₇N₆O₂, 537.2978 [M+H]⁺; found, 537.2941.

HPLC purity (method B): 94.2% (t_{R}=17.49 min).

### Example 51: N-(2-{[2-(Dimethylamino)ethyl](methyl)amino}-5-{4-[(3-hydroxy-cyclobutyl)amino]-6-phenylfuro[2,3-d]pyrimidin-5-yl}phenyl)prop-2-enamide (51)

Compound 70w (see Scheme I) was prepared following the procedure described in Li et al., J. Med. Chem. 66, 2566-88 (2023), Supporting Information.

To a solution of 70w (66 mg, 0.16 mmol, 1 equiv) in dichloromethane (0.7 mL) were added acrylic acid (11 μL, 0.16 mmol, 1.1 equiv) and EDCI (40 mg, 0.21 mmol, 1.5 equiv); then, the reaction mixture was stirred at room temperature. After stirring for 16 h, the reaction mixture was concentrated in vacuo and purified by flash column chromatography (5% methanol in dichloromethane) to yield the title compound (16 mg, 0.03 mmol, 22%) as a white solid.

¹H NMR (600 MHz, DMSO-d₆) δ 10.32 (s, 1H), 8.42 (d, J=2.4 Hz, 1H), 8.34 (s, 1H), 7.56 (d, J=6.6 Hz, 2H), 7.45 (d, J=8.1 Hz, 1H), 7.40-7.31 (m, 3H), 7.21 (dd, J=8.1, 2.4 Hz, 1H), 6.47 (dd, J=17.4, 10.5 Hz, 1H), 6.26 (dd, J=17.4, 2.4 Hz, 1H), 5.80 (dd, J=10.5, 2.4 Hz, 1H), 5.30 (d, J=6.6 Hz, 1H), 5.02 (s, 1H), 4.50-4.42 (m, 1H), 4.12-4.05 (m, 1H), 2.93 (t, J=6.0 Hz, 2H), 2.78 (s, 3H), 2.40 (t, J=6.0 Hz, 2H), 2.22 (s, 6H), 2.17-2.11 (m, 2H), 1.95-1.87 (m 2H).

¹³C NMR (150 MHz, DMSO-d₆) δ 164.4, 163.4, 156.5, 154.0, 145.4, 143.8, 134.3, 131.6, 129.1, 128.8, 128.7, 127.3, 126.6, 126.0, 125.4, 122.7, 121.5, 115.1, 102.6, 63.0, 56.7, 55.6, 45.5, 45.4, 42.1, 41.2.

LRMS (ESI) m/z: 527.3 [M+H]⁺. HRMS (ESI) m/z: calcd for C₃₀H₃₅N₆O₃, 527.2771 [M+H]⁺; found, 527.2781.

HPLC purity (method B): 98.2% (t_{R}=14.10 min).

### Example 52: N-(2-{[2-(Dimethylamino)ethyl](methyl)amino}-5-{4-[(4-hydroxy-cyclohexyl)amino]-6-phenylfuro[2,3-d]pyrimidin-5-yl}phenyl)prop-2-enamide

Compound 52 was prepared following the procedure as described in Example 26 using 4-aminocyclohexan-1-ol.

¹H NMR (400 MHz, CDCl₃) δ 10.42 (br s, 1H), 8.65 (d, J=2.0 Hz, 1H), 8.39 (s, 1H), 7.56-7.51 (m, 2H), 7.32 (d, J=8.0 Hz, 1H), 7.27-7.21 (m, 3H), 7.13 (dd, J=8.0, 1.2 Hz, 1H), 6.46 (dd, J=16.8, 1.6 Hz, 1H), 6.35 (dd, J=16.8, 10.0 Hz, 1H), 5.76 (dd, J=10.0, 1.6 Hz, 1H), 5.16 (d, J=7.2 Hz, 1H), 4.14 (bs, 1H), 3.65-3.58 (m, 1H), 2.97-2.93 (m, 2H), 2.78 (s, 3H), 2.43 (t, J=5.2 Hz, 2H), 2.33 (s, 6H), 1.75-1.62 (m, 8H), 1.25 (br s, 1H).

LRMS (ESI) m/z: 555.3 [M+H]⁺.

HPLC purity (method B): 96.1% (t_{R}=15.63 min).

### Example 53: N-[2-{[2-(Dimethylamino)ethyl](methyl)amino}-5-(4-{[(1R,2S)-2-hydroxycyclohexyl]amino}-6-phenylfuro[2,3-d]pyrimidin-5-yl)phenyl]prop-2-enamide

Compound 53 was prepared following the procedure as described in Example 26 using (1S,2R)-2-aminocyclohexan-1-ol.

¹H NMR (300 MHz, CDCl₃) δ 10.39 (br s, 1H), 8.67 (s, 1H), 8.35 (s, 1H), 7.61-7.55 (m, 2H), 7.32-7.21 (m, 4H), 7.14 (dd, J=8.4, 2.1 Hz, 1H), 6.45 (d, J=17.1 Hz, 1H), 6.32 (dd, J=17.1, 9.9 Hz, 1H), 5.75 (d, J=9.9 Hz, 1H), 5.37 (d, J=7.8 Hz, 1H), 4.35-4.27 (m, 1H), 3.90 (br s, 1H), 3.60 (br s, 1H), 2.98-2.93 (m, 2H), 2.78 (s, 3H), 2.41 (t, J=5.1 Hz, 2H), 2.32 (s, 6H), 1.58-1.51 (m, 3H), 1.48-1.41 (m, 2H), 1.39-1.23 (m, 3H).

LRMS (ESI) m/z: 555.3 [M+H]⁺.

HPLC purity (method B): 93.7% (t_{R}=16.24 min).

### Example 54: N-[2-{[2-(Dimethylamino)ethyl](methyl)amino}-5-(4-{[(1R,2R)-2-hydroxycyclohexyl]amino}-6-phenylfuro[2,3-d]pyrimidin-5-yl)phenyl]prop-2-enamide

Compound 54 was prepared following the procedure as described in Example 26 using (1R,2R)-2-aminocyclohexan-1-ol.

¹H NMR (300 MHz, CDCl₃) δ 10.42 (br s, 1H), 8.79 (s, 1H), 8.35 (s, 1H), 7.63-7.56 (m, 2H), 7.32-7.27 (m, 4H), 7.13 (d, J=8.1 Hz, 1H), 6.44 (dd, J=17.1, 1.8 Hz, 1H), 6.32 (dd, J=17.1, 99 Hz, 1H), 5.75 (dd, J=9.9, 1.8 Hz, 1H), 5.17 (br s, 1H), 4.99 (d, J=6.6 HZ, 1H), 3.86 (br s, 1H), 3.20-3.24 (m, 1H), 2.92-2.96 (m, 2H), 2.80 (s, 3H), 2.43 (t, J=5.1 Hz, 2H), 2.33 (s, 6H), 2.00 (d, J=10.2 Hz, 1H), 1.87 (d, J=10.2 Hz, 1H), 1.69-1.63 (m, 1H), 1.55 (d, J=12.9 Hz, 1H), 1.43-1.22 (m, 2H), 1.17-1.04 (m, 1H), 0.92-0.80 (m, 1H).

LRMS (ESI) m/z: 555.3 [M+H]⁺.

HPLC purity (method B): 96.0% (t_{R}=15.91 min).

### Example 55: N-(2-{[2-(Dimethylamino)ethyl](methyl)amino}-5-{4-[(4-hydroxy-cyclohexyl)amino]-6-phenylfuro[2,3-d]pyrimidin-5-yl}phenyl)prop-2-enamide

Compound 55 was prepared following the procedure as described in Example 26 using 4-aminocyclohexan-1-ol.

¹H NMR (300 MHz, CDCl₃) δ 10.30 (br s, 1H), 8.78 (s, 1H), 8.38 (s, 1H), 7.60-7.57 (m, 2H), 7.32-7.23 (m, 4H), 7.10 (dd, J=7.8, 2.1 Hz, 1H), 6.43 (dd, J=14.1, 9.3 Hz, 1H), 6.34 (dd, J=14.1, 9.3 Hz, 1H), 5.14 (dd, J=9.3, 2.1 Hz, 1H), 4.88 (d, J=7.5 Hz, 1H), 4.04-3.92 (m, 1H), 3.59-3.48 (m, 1H), 2.92 (t, J=4.5 Hz, 2H), 2.79 (s, 3H), 2.43 (t, J=4.5 Hz, 2H), 2.33 (s, 6H), 2.04 (d, J=9.6 Hz, 2H), 1.85-1.79 (m, 2H), 1.47-1.34 (m, 2H), 1.25 (bs, 1H), 1.18-1.05 (m, 2H).

LRMS (ESI) m/z: 555.3 [M+H]+.

HPLC purity (method B): 91.7% (t_{R}=14.79 min).

### Example 56: N-(2-{[2-(Dimethylamino)ethyl](methyl)amino}-5-{4-[((1R,2R)-2-hydroxycyclopentyl)amino]-6-phenylfuro[2,3-d]pyrimidin-5-yl}phenyl)prop-2-enamide (56)

Compound 70u (see Scheme I) was prepared following the procedure described in Li et al., J. Med. Chem. 66, 2566-88 (2023), Supporting Information.

To a solution of 70u (54 mg, 0.11 mmol, 1 equiv) in dichloromethane (1.5 mL) were added acrylic acid (9 μL, 0.13 mmol, 1.2 equiv) and EDCI (32 mg, 0.17 mmol, 1.5 equiv); then, the reaction mixture was stirred at room temperature. After stirring for 16 h, the reaction mixture was concentrated in vacuo and purified by flash column chromatography (5% methanol in dichloromethane) to yield the title compound (38 mg, 0.07 mmol, 63%) as colorless oil.

¹H NMR (600 MHz, DMSO-d₆) δ 10.30 (s, 1H), 8.45 (d, J=2.4 Hz, 1H), 8.35 (s, 1H), 7.55 (d, J=6.6 Hz, 2H), 7.45 (d, J=8.1 Hz, 1H), 7.38-7.30 (m, 3H), 7.20 (dd, J=8.1, 2.4 Hz, 1H), 6.46 (dd, J=16.8, 10.2 Hz, 1H), 6.24 (dd, J=16.8, 1.8 Hz, 1H), 5.80 (dd, J=10.2, 1.8 Hz, 1H), 4.90-4.80 (m, 2H), 4.16-4.10 (m, 1H), 3.74-3.67 (m, 1H), 2.90 (t, J=6.0 Hz, 2H), 2.76 (s, 3H), 2.40 (t, J=6.0 Hz, 2H), 2.22 (s, 6H), 2.03-1.95 (m, 1H), 1.65-1.55 (m 1H), 1.54-1.46 (m, 1H), 1.42-1.34 (m, 1H), 1.32-1.16 (m, 1H).

¹³C NMR (150 MHz, DMSO-d₆) δ 164.3, 163.4, 156.7, 154.1, 145.4, 143.6, 134.6, 131.6, 129.1, 128.8, 128.7, 127.4, 126.8, 125.8, 125.2, 122.8, 121.0, 115.0, 102.5, 75.8, 58.7, 56.7, 55.6, 45.5, 41.2, 31.6, 29.4, 20.2.

LRMS (ESI) m/z: 541.5 [M+H]⁺. HRMS (ESI) m/z: calcd for C₃₁H₃₇N₆O₃, 541.2927 [M+H]⁺; found, 541.2907.

HPLC purity (method B): 95.2% (t_{R}=14.85 min).

### Example 57: N-(2-{[2-(Dimethylamino)ethyl](methyl)amino}-5-{4-[(4-methoxy-cyclohexyl)amino]-6-phenylfuro[2,3-d]pyrimidin-5-yl}phenyl)prop-2-enamide (57)

Compound 57 was prepared following the procedure as described in Example 26 using 4-methoxycyclohexan-1-amine.

¹H NMR (400 MHz, CDCl₃) δ 10.31(brs, NH), 8.80 (s, 1H), 8.38 (s, 1H), 7.60-7.57 (m, 2H), 7.32-7.23 (m, 4H), 7.11 (dd, J=8.4, 2.0 Hz, 1H), 6.43 (dd, J=16.8, 1.6 Hz, 1H), 6.33 (dd, J=17.2, 10.0 Hz, 1H), 5.74 (dd, J=10.0, 1.6 Hz, 1H), 4.90 (d, J=7.6 Hz, 1H), 4.00 (t, J=3.6 Hz, 1H), 3.29 (s, 3H), 3.07-3.03 (m, 1H), 2.92 (t, J=5.2 Hz, 2H), 2.80 (s, 3H), 2.42 (t, J=5.2 Hz, 2H), 2.30 (s, 6H), 2.03 (d, J=9.6 Hz, 2H), 1.80 (d, J=9.6 Hz, 2H), 1.42-1.33 (m, 2H), 1.06 (d, J=10.4 Hz, 2H).

LRMS (ESI) m/z: 569.3 [M+H]⁺.

UPLC purity: 97.3% (t_{R}=1.91 min).

### Example 58: N-[5-(4-{[4-(Acetylamino)cyclohexyl]amino}-6-phenylfuro[2,3-d]pyrimidin-5-yl)-2-{[2-(dimethylamino)ethyl](methyl)amino}phenyl]prop-2-enamide (58)

Compound 58 was prepared following the procedure as described in Example 26 using N-(4-aminocyclohexyl)acetamide.

¹H NMR (300 MHz, CDCl₃): δ 10.32 (brs, NH), 8.78 (s, 1H), 8.36 (s, 1H), 7.59 (dd, J=8.1, 2.4 Hz, 2H), 7.31-7.24 (m, 4H), 7.09 (dd, J=7.8, 1.5 Hz, 1H), 6.39 (t, J=17.1 Hz, 2H), 5.74 (t, J=3.0 Hz, 1H), 5.31 (d, J=8.1 Hz, 1H), 4.81 (d, J=7.5 Hz, 1H), 3.97-3.92 (m, 1H), 3.68-3.61 (m, 1H), 2.93 (s, 2H), 2.81 (s, 3H), 2.46 (s, 2H), 2.35 (s, 6H), 2.03 (d, J=10.5 Hz, 2H), 1.91 (d, J=19.2 Hz, 5H), 1.31-1.18 (m, 2H), 1.05 (dd, J=23.1, 11.1 Hz, 2H).

LRMS (ESI) m/z: 596.4 [M+H]⁺.

UPLC purity: 97.6% (t_{R}=1.71 min).

### Example 59: (2E)-N-{3-[4-(Cyclopentylamino)-6-phenylfuro[2,3-d]pyrimidin-5-yl]phenyl}-4-(pyrrolidin-1-yl)but-2-enamide (59)

Compound 59 was prepared following the procedure as described in Example 19 using (2E)-4-bromobut-2-enoic acid and pyrrolidine

¹H NMR (300 MHz, CDCl₃) δ 8.40 (s, 1H), 7.89 (d, J=7.8 Hz, 1H), 7.64 (d, J=10.4 Hz, 2H), 7.56-7.49 (m, 3H), 7.31-7.06 (m, 4H), 7.04-6.97 (m, 1H), 6.18 (dd, J=13.8, 1.5 Hz, 1H), 4.75 (d, J=7.5 Hz, 1H), 4.43-4.37 (m, 1H), 3.33 (dd, J=8.0, 1.5 Hz, 2H), 2.63-2.59 (m, 4H), 1.96-1.83 (m, 6H), 1.49-1.38 (m, 4H), 1.25-1.19 (m, 2H).

LRMS (ESI) m/z: 508.4 [M+H]⁺.

HPLC purity (method B): 99.2% (t_{R}=17.92 min).

### Example 60: (2E)-N-{3-[4-(Cyclopentylamino)-6-phenylfuro[2,3-d]pyrimidin-5-yl]phenyl}-4-(morpholin-4-yl)but-2-enamide

Compound 60 was prepared following the procedure as described in Example 19 using (2E)-4-bromobut-2-enoic acid and morpholine.

¹H NMR (400 MHz, CDCl₃) δ 8.39 (s, 1H), 7.93 (d, J=8.0 Hz, 1H), 7.69 (s, 1H), 7.59 (s, 1H), 7.54-7.50 (m, 3H), 7.27-7.18 (m, 4H), 7.02-6.95 (m, 1H), 6.16 (dd, J=14.0, 1.6 Hz, 1H), 4.74 (d, J=7.2 Hz, 1H), 4.40-4.39 (m, 1H), 3.72 (t, J=4.4 Hz, 4H), 3.16 (dd, J=6.0, 1.2 Hz, 2H), 2.48 (t, J=4.4 Hz, 4H), 1.95-1.90 (m, 2H), 1.58-1.43 (m, 4H), 1.25-1.21 (m, 2H).

LRMS (ESI) m/z: 524.4 [M+H]⁺.

HPLC purity (method B): 95.7% (t_{R}=17.72 min).

### Example 61: N-{3-[4-(Azetidin-1-yl)-6-phenylfuro[2,3-d]pyrimidin-5-yl]phenyl}prop-2-enamide

Compound 61 was prepared following the procedure as described in Example 19 using azetidine.

¹H NMR (400 MHz, CDCl₃) δ 8.38 (s, 1H), 7.96 (d, J=6.6 Hz, 1H), 7.34-7.55 (m, 4H), 7.10-7.25 (m, 4H), 6.46 (dd, J=16.8, 1.2 Hz, 1H), 6.25 (dd, J=16.8, 10.2 Hz, 1H), 5.80 (dd, J=10.2, 1.2 Hz, 1H), 3.55-3.75 (m, 4H), 2.08-2.13 (m, 2H).

LRMS (ESI) m/z: 397.1 [M+H]⁺.

HPLC purity (method B): 97.5% (t_{R}=22.20 min).

### Example 62: N-(3-{6-[4-(4-Methylpiperazin-1-yl)phenyl]-4-(pyrrolidin-1-yl)furo[2,3-d]pyrimidin-5-yl}phenyl)prop-2-enamide

Compound 62 was prepared following the procedure as described in Example 13 using pyrrolidine.

¹H NMR (300 MHz, CDCl₃) δ 8.26 (s, 1H), 7.94 (d, J=8.4 Hz, 1H), 7.48 (s, 1H), 7.37 (dd, J=8.4, 7.8 Hz, 1H), 7.25 (s, 1H), 7.20 (d, J=9.0 Hz, 2H), 7.10 (d, J=7.8 Hz, 1H), 6.68 (d, J=9.0 Hz, 2H), 6.39 (dd, J=16.7, 1.2 Hz, 1H), 6.28 (dd, J=16.7, 10.1 Hz 1H), 5.72 (dd, J=10.1, 1.2 Hz, 1H), 3.16-3.13 (m, 4H), 3.06-3.04 (m, 4H), 2.48-2.45 (m, 4H), 2.27 (s, 3H), 1.64-1.59 (m, 4H).

LRMS (ESI) m/z: 509.5 [M+H]⁺.

HPLC purity (method B): 97.1% (t_{R}=14.55 min).

### Example 63: N-(3-{4-(Azetidin-1-yl)-6-[4-(4-methylpiperazin-1-yl)phenyl]furo[2,3-d]pyrimidin-5-yl}phenyl)prop-2-enamide

Compound 63 was prepared following the procedure as described in Example 19 using azetidine.

¹H NMR (300 MHz, CDCl₃) δ 8.35 (s, 1H), 8.03 (d, J=7.8 Hz, 1H), 7.80 (s, 1H), 7.46 (dd, J=7.8, 7.8 Hz, 1H), 7.32 (d, J=5.7 Hz, 1H), 7.33-7.27 (m, 3H), 6.73 (d, J=8.7 Hz, 2H), 6.47 (d, J=16.6 Hz, 1H), 6.30 (dd, J=16.6, 10.1 Hz, 1H), 5.79 (d, J=10.1 Hz, 1H), 3.64 (br, 4H), 3.22-3.19 (m, 4H), 2.53-2.50 (m, 4H), 2.32 (s, 3H), 2.14-2.03 (m, 2H).

LRMS (ESI) m/z: 495.5 [M+H]⁺.

HPLC purity (method B): 98.1% (t_{R}=21.79 min).

### Example 64: N-(2-{[2-(Dimethylamino)ethyl](methyl)amino}-5-[6-phenyl-4-(pyrrolidin-1-yl)furo[2,3-d]pyrimidin-5-yl]phenyl)prop-2-enamide

Compound 64 was prepared following the procedure as described in Example 26 using pyrrolidine.

¹H NMR (400 MHz, CDCl₃) δ 10.22 (s, 1H), 8.63 (s, 1H), 8.37 (d, J=4.0 Hz, 1H), 7.45 (t, J=4.0 Hz, 2H), 7.31-7.28 (m, 1H), 7.21-7.20 (m, 3H), 7.08-7.06 (m, 1H), 6.41 (d, J=16.0 Hz, 1H), 6.31 (dd, J=16.0, 9.6 Hz, 1H), 5.72 (d, J=9.6 Hz, 1H), 3.25-3.05 (m, 4H), 2.95-2.85 (m, 2H), 2.78 (s, 3H), 2.35-2.40 (m, 2H), 2.30 (s, 6H), 1.65-1.75 (m, 4H).

LRMS (ESI) m/z: 511.3 [M+H]⁺.

HPLC purity (method B): 94.3% (t_{R}=15.50 min).

### Example 65: N-(5-[4-(Azetidin-1-yl)-6-phenylfuro[2,3-d]pyrimidin-5-yl]-2-{[2-(dimethylamino)ethyl](methyl)amino}phenyl)prop-2-enamide

Compound 65 was prepared following the procedure as described in Example 26 using azetidine.

¹H NMR (400 MHz, CDCl₃) δ 10.24 (s, 1H), 8.65 (s, 1H), 8.39 (s, 1H), 7.48-7.46 (m, 2H), 7.31 (d, J=8.0 Hz, 1H), 7.22-7.20 (m, 3H), 7.08 (dd, J=8.0, 2.0 Hz, 1H), 6.42 (d, J=15.6 Hz, 1H), 6.31 (dd, J=15.6, 10.8 Hz, 1H), 5.73 (d, J=10.8 Hz, 1H), 3.80-3.60 (m, 4H), 2.92 (t, J=5.4 Hz, 2H), 2.80 (s, 3H), 2.37 (t, J=5.4 Hz, 2H), 2.31 (s, 6H), 2.10 (quint, J=7.6 Hz, 2H).

LRMS (ESI) m/z: 497.3 [M+H]⁺.

HPLC purity (method B): 93.6% (t_{R}=14.08 min).

EGFR^{WT} and EGFR^{L858R/T790M} Enzyme Inhibition Assays

Kinase-Glo Plus Luminescent Kinase Assay (Promega) was used to test the inhibition potential of compounds of this invention for GST-EGFR (G696-G1022) wild-type and L858R/T790M double-mutant recombinant proteins. Detailed methodology for carrying out the enzyme inhibition assay is described in Lin et al., J. Med. Chem. 2019, 62, 10108-23.

Comparative Compound DBPR112 was tested together with compounds of this inventions. The structure of DBPR112 is shown below:

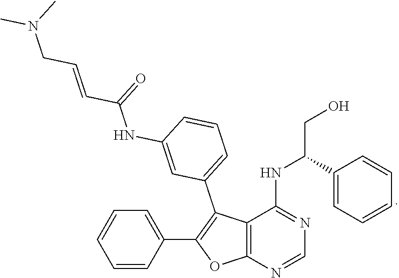 [SMILES: CN(C)C/C=C/C(=O)Nc1cccc(-c2c(-c3ccccc3)oc3ncnc(N[C@H](CO)c4ccccc4)c23)c1]

DBPR112 was found to inhibit the EGFR^{WT} to a greater degree than the mutant EGFR kinase (EGFR^{WT} IC₅₀=15 nM vs EGFR^{L858R/T790M} IC₅₀=48 nM), showing no selectivity of inhibiting EGFR^{L858R/T790M} over EGFR^{WT}. On the other hand, compounds of this invention showed a selectivity of 1.5 to 61. Compound 19 has a selectivity of 61, inhibiting EGFR^{L858R/T790M} 61 folds greater than EGFR^{WT}. Compounds 1, 16, 20, and 21 have a selectivity of 10.3, 13, 15.8, and 26.8, respectively.

### Cell Proliferation Assay and Western Blot Analysis

A431 (wild-type EGFR overexpressing) and H1975 (double-mutant overexpressing) cells were exposed to test compounds for 96 h and the cell proliferation was determined by the MTS method. Western blot analysis was performed by exposing the cells (A431 and H1975) to the test compound for 1 h. Drug-treated cells were lysed and subjected to western blot analysis. For detailed procedure, see Lin et al., J. Med. Chem. 2019, 62, 10108-23; and Lin et al., Anal. Biochem. 2008, 377, 89-94.

The results are shown in Table 2 below.

TABLE 2

| Compound | A431 IC₅₀ | H1975 IC₅₀ |
|---|---|---|
| 1 | + | ++ |
| 2 | + | ++ |
| 4 | + | ++ |
| 13 | ++ | +++ |
| 14 | + | ++ |
| 15 | + | +++ |
| 19 | + | ++ |
| 21 | + | ++ |
| 22 | + | ++ |
| 23 | + | ++ |
| 24 | ++ | +++ |
| 25 | + | ++ |
| 26 | ++ | ++ |
| 27 | + | ++ |
| 28 | + | ++ |
| 30 | + | ++ |
| 31 | + | ++ |
| 33 | ++ | ++ |
| 34 | ++ | +++ |
| 35 | ++ | ++ |
| 36 | ++ | ++ |
| 37 | ++ | ++ |
| 38 | ++ | ++ |
| 40 | + | ++ |
| 41 | ++ | +++ |
| 42 | ++ | +++ |
| 43 | ++ | +++ |
| 44 | + | ++ |
| 45 | ++ | ++ |
| 46 | ++ | ++ |
| 47 | ++ | +++ |
| 48 | ++ | +++ |
| 49 | ++ | +++ |
| 50 | + | ++ |
| 51 | ++ | +++ |
| 52 | ++ | +++ |
| 53 | ++ | +++ |
| 55 | ++ | +++ |
| 61 | ++ | +++ |

Note:
“+++” indicates an IC₅₀ value less than 200 nM; “++” indicates an IC₅₀ value between 200 nM and 5,000 nM; and “+” indicates an IC₅₀ value greater than 5,000 nM. A431 is wild-type EGFR overexpressing tumor cells and HCC1975 is double-mutant (EGFRL858R/T790M) overexpressing tumor cells.

The results demonstrate that compounds of this invention selectively inhibit cell proliferation of double mutant EGFR tumor cells.

### In Vivo BaF3 EGF R^{L858R/T790M} Xenograft Study

Adult male nude mice (Nu-Fox1nu) purchased from BioLasco, Taiwan Co., Ltd. were used for this study. Animals had access to food and water ad libitum. Institutional Animal Care and Use Committees (IACUC) of the National Health Research Institutes approved the study using animals. EGFR^{L858R/790M} expressing human BaF3 cells were cultured in RPMI-1640 supplemented with 10% (v/v) fetal bovine serum (FBS) at 37° C. in a humidified atmosphere consisting of 5% CO₂. Each 9-week-old nude mouse was inoculated subcutaneously with 1×10⁶ BaF3 EGFR^{L858R/T790M} cells in 50% Matrigel (Becton Bickinson) in 0.1 mL injection volume via a 24-gauge needle. Tumor volume was measured with the help of an electronic caliper and calculated using the formula, length×width²×0.5. When the size of a growing tumor reached ˜280 mm³, the tumor-bearing mice were administered compound 26 and 47 (dissolved in 20% 2-hydroxypropyl-β-cyclodextrin in water) by oral gavage 5 days a week for 2 consecutive weeks at 100 mg/kg doses. AZD9291 (i.e., osimertinib) was administered orally at 10 mg/kg doses in the same regimen for comparison. The control group received vehicle alone (20% 2-hydroxypropyl-β-cyclodextrin in water) by oral gavage. The group that received 100 mg/kg of Compound 26 showed 12% TGI at the end of the treatment, while the group administered 100 mg/kg of Compound 47 showed 89% TGI.

The antiproliferative activities of Compounds 26 and 47 were confirmed in BaF3 cell lines overexpressed with EGFR^{L858R/T790M} mutant kinase. Both compounds showed potent antiproliferative activity with CC₅₀ values of 26 and 20 nM, respectively, similar to that of AZD9291, which had a CC₅₀ of 14 nM. The results suggested that both compounds inhibit double mutant EGFR at the cellular level.

Further, to confirm the ability of Compounds 26 and 47 to inhibit mutant EGFR selectively inside the cells, A431 and H1975 NSCLC cells were treated with the compounds for 1 h and then the cell extracts were analyzed using western blot for downstream signaling molecule modulation. Treatment with Compounds 26 and 47 resulted in lower levels of tyrosine¹⁰⁶⁸ phosphorylated EGFR [pEGFR(Tyr¹⁰⁶⁸)] in the H1975 cell line but not in the A431 cell line. An active EGFR kinase enzyme autophosphorylates the EGFR tyrosine¹⁰⁶⁸ residue. Lower levels of pEGFR(Tyr¹⁰⁶⁸) in H1975 indicate target (EGFR^{L858R/T790M}) inhibition in the cell lines. Levels of pEGFR(Tyr¹⁰⁶⁸) were not affected in A431 cell line, suggesting that the target (EGFR^{WT}) is not inhibited in this cell line. This further confirms that Compounds 26 and 47 are mutant-selective EGFR inhibitors with EGFR^{WT} sparing activity.

### In Vivo H1975 Xenograft Study

To assess the antitumor activity of Compound 47, five-week-old male nude mice (National Laboratory Animal Center, NLAC, Taipei, Taiwan) were subcutaneously injected with 1×10⁷ H1975 cells. When the tumor sizes reached 100 mm³, mice were divided into four groups randomly (control, AZD9291 10 mg/kg, Compound 47 10 and 30 mg/kg groups, with 9-10 mice in each group). Mice in control groups were dosed with the vehicle (20% 2-hydroxypropyl-β-cyclodextrin, HP-3-CD) by oral administration (po) once daily (qd); AZD9291 group was dosed with 10 mg/kg of AZD9291 po, qd; Compound 47 groups were dosed with 10 or 30 mg/kg of Compound 47 po, qd. The tumor size and body weight were measured twice per week during the experiment, and the tumor volume (mm³) was calculated as mentioned above. Tumor growth inhibition (% TGI)=[1−(Tₜ−T₀)/(Cₜ−C₀)×100], where C₀ and Cₜ are mean tumor volumes of the control group by first data point and day t, respectively, while T₀ and Tₜ are mean tumor volumes of the treatment group by first data point and day t, respectively. This study was approved by Animal Use and Management Committee of Taipei Medical University (IACUC number LAC-2017-0444).

The group that received 10 mg/kg of Compound 47 showed 74.9% TGI, while the group administered 30 mg/kg showed 97.5% TGI at the end of the treatment. The results demonstrate that Compound 47 effectively suppresses the growth of NSCLC with EGFR double mutation. Moreover, drug treatment did not result in overt body weight loss (<5% change in body weight) during drug treatment, suggesting overall good tolerance for the drug. The in vivo pharmacodynamics experiments demonstrate the utility of Compound 47 as an agent to overcome EGFR double-mutant overexpressing non-small cell lung cancer.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A compound of Formula (I): 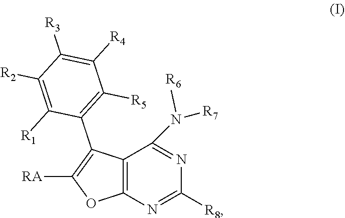
in which
each of R₁, R₄, R₅, and R₈, independently, is H, D, halo, or C₁-C₆ alkyl;
R₂ is C₂-C₆ alkenylamido;
R₃ is H, D, halo, C₁-C₆ alkyl, C₂-C₆ alkenylamido, C₁-C₆ alkylamino, C₁-C₁₀ dialkylamino, 5- to 6-membered heterocycloalkyl, C₁-C₆ alkoxy, or 5- to 6-membered heterocycloalkoxy;
R₆ is H or C₁-C₆ alkyl; R₇ is C₁-C₆ alkyl, C₃-C₆ cycloalkyl, 5- to 6-membered heterocycloalkyl, aryl, C₇-C₁₀ aralkyl, heteroaryl, or C₅-C₁₀ heteroaralkyl; or R₆ and R₇, together with the nitrogen atom they bond to, form a 4- to 7-membered heterocycloalkyl;
RA is H, aryl, or heteroaryl;
alkyl is unsubstituted or substituted with one or more groups selected from D, halo, hydroxyl, amino, CN, carboxyl, C₁-C₆ carboxylate, C₁-C₆ alkylamino, C₁-C₁₀ dialkylamino, C₁-C₆ alkoxy, aryl, heteroaryl, aryloxy, C₅-C₆ cycloalkyl, or 5- to 6-membered heterocycloalkyl; and
each of amino, alkylamino, dialkylamino, alkenylamido, alkoxy, cycloalkyl, heterocycloalkyl, heterocycloalkoxy, aryl, aralkyl, aryloxy, heteroaryl and heteroaralkyl, independently, is unsubstituted or substituted with one or more groups selected from D, halo, hydroxyl, amino, CN, sulfamoyl, C₁-C₆ alkyl, C₁-C₆ alkoxy, C₂-C₆ alkenyl, C₂-C₆ alkynyl, C₁-C₆ alkylamino, C₁-C₁₀ dialkylamino, C₁-C₆ carboxylate, C₁-C₆ amido, 5- or 6-membered heterocycloalkyl, aryl, and heteroaryl.

2. The compound of claim 1, wherein the compound is a compound of Formula (II): 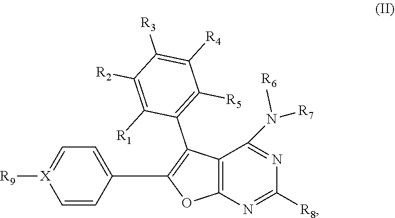
in which X is N or C; and R₉ is absent, H, halo, or 5- to 6-membered heterocycloalkyl, provided that when X is N, R₉ is absent.

3. The compound of claim 1, wherein R₂ is

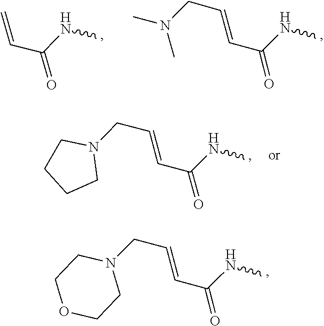

4. The compound of claim 3, wherein R₂ is

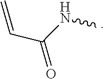

5. The compound of claim 2, wherein R₂ is

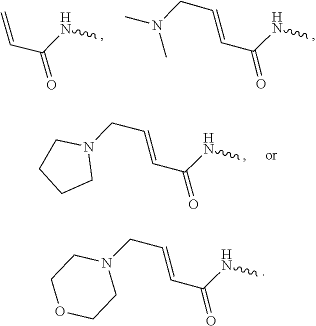

6. The compound of claim 5, wherein R₂ is

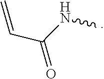

7. The compound of claim 1, wherein R₃ is H,

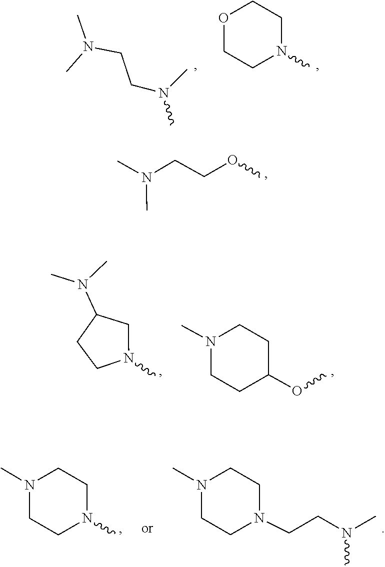

8. The compound of claim 7, wherein R₃ is

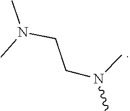

9. The compound of claim 1, wherein R₂ is 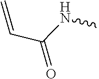
and R₃ is

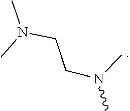

10. The compound of claim 2, wherein R₃ is H

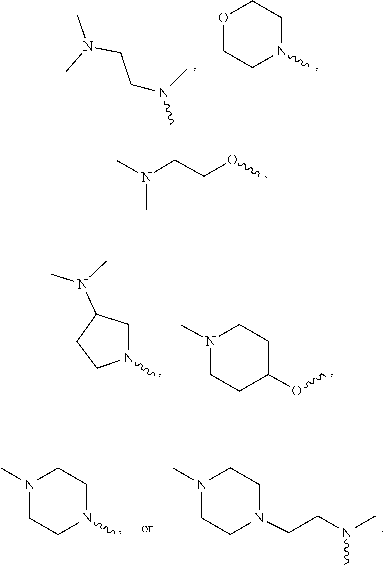

11. The compound of claim 10, wherein R₃ is

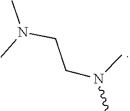

12. The compound of claim 1, wherein R₆ is H and R₇ is CH₃, CH₂CH₃, 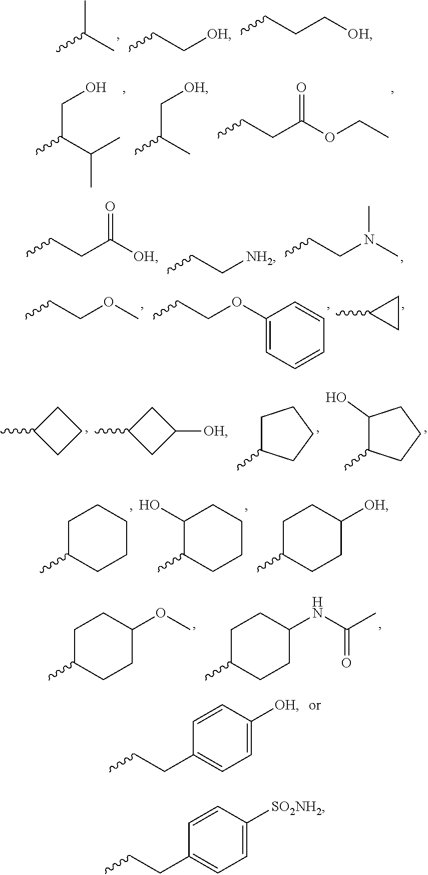
or; or R₆ and R₇, together with the nitrogen atom they bond to, are azetidinyl, pyrrolidinyl, or piperidinyl.

13. The compound of claim 12, wherein R₇ is

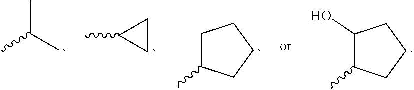

14. The compound of claim 2, wherein R₆ is H and R₇ is CH₃, CH₂CH₃, 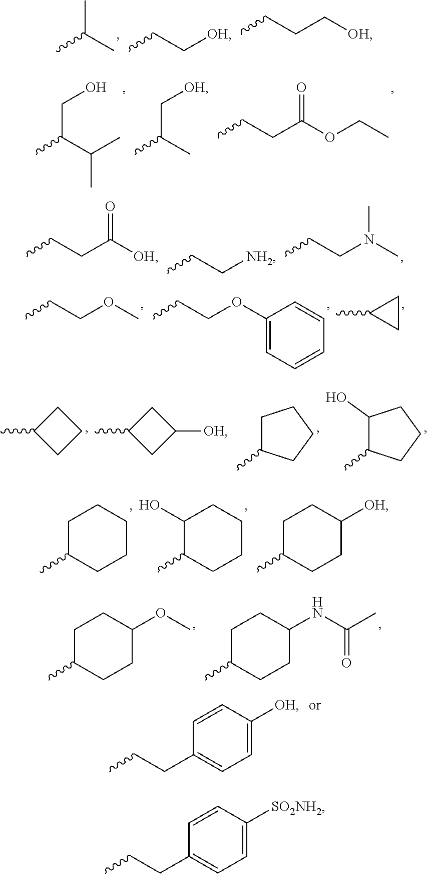
or R₆ and R₇, together with the nitrogen atom they bond to, are azetidinyl, pyrrolidinyl, or piperidinyl.

15. The compound of claim 13, wherein R₇ is

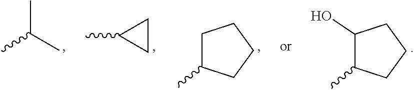

16. The compound of claim 2, wherein X is C and R₉ is H, F, N(CH₃)₂,

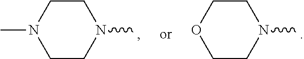

17. The compound of claim 2, wherein X is C; each of R₁, R₄, R₅, R₆, R₈, and R₉ is H; R₂ is 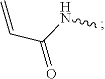
R₃ is 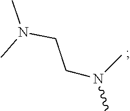
and R₇ is

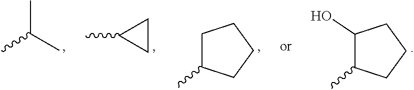

18. The compound of claim 1, wherein the compound is one of Compounds 1-65.

19. A pharmaceutical composition comprising a compound of claim 1 and a pharmaceutically acceptable carrier.

20. A method of treating cancer comprising administering to a subject in need thereof an effective amount of a compound of claim 1.
